SEQUENCE LISTING (63 sequences)

SEQ ID NO: 1 - DNA, 1840 bp, Homo sapiens
  CDS (116)..(1264)
ctaactttgggaactcgtatagacccagcgtcgctccccgcggtgcctcgcctccactttggtttcccgcgtcctgcccgctctcttcggtgcctcctcttcctccgggacaaggatggaggatctctttagcccctcaattctgccgccggcgcccaacatttccgtgcccatcttgctgggctggggtctcaacctgaccttggggcaaggagcccctgcctctgggccgcccagccgccgcgtccgcctggtgttcctgggggtcatcctggtggtggcggtggcaggcaacaccacagtgctgtgccgcctgtgcggcggcggcgggccctgggcgggccccaagcgtcgcaagatggacttcctgctggtgcagctggccctggcggacctgtacgcgtgcgggggcacggcgctgtcacagctggcctgggaactgctgggcgagccccgcgcggccacgggggacctggcgtgccgcttcctgcagctgctgcaggcatccgggcggggcgcctcggcccacctcgtggtgctcatcgccctcgagcgccggcgcgcgccaggcgcgccactctccgcccgagcctggccgggggagcgtcgctgccacgggatcttcgcgcccctgccgcgctggcacctgcaggtctacgcgttctacgaggccgtcgcgggcttcgtcgcgcctgttacggtcctgggcgtcgcttgcggccacctactctccgtctggtggcggcaccggccgcaggcccccgcggctgcagcgccctggtcggcgagcccaggtcgagcccctgcgcccagcgcgctgccccgcgccaaggtgcagagcctgaagatgagcctgctgctggcgctgctgttcgtgggctgcgagctgccctactttgccgcccggctggcggccgcgtggtcgtccgggcccgcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttctaggtgcttggtggtcagagacgggtcatctgtcgctaaggcgcaacctccagggaactcgaggcctgccagggtctgtccagatcacaaggggcaggagagtctgtgagagagtgacactgaagttgtccccttcctccactctcctattcccttctcatgtttacatttccctatgctcttccagtttctcttcttccctacagttcctctcatatctccccatttggagacagtgagccactggaaagttgtaaaaacaaaaacagttatttttgcagttttctttcacgcatttatagtgctctggataatgccatttatttttgctgattacccaactttcagtatttgctgtgttatcatctgtatttacttattttgaatcgtgcttaaatcaaatgtaccttcagcacctgcaagtttgccttttctttccaggaggaaaatccccacgttgctctccctggggagtctgagaattataccagtgctgtcagaaatgtaatcatgctgtcatttcagagccacagagtatttataaaataaaaacctttcccacggaaaaaaaaaaaa

SEQ ID NO: 2 - protein, 383 aa, Homo sapiens
MEDLFSPSILPPAPNISVPILLGWGLNLTLGQGAPASGPPSRRVRLVFLGVILVVAVAGNTTVLCRLCGGGGPWAGPKRRKMDFLLVQLALADLYACGGTALSQLAWELLGEPRAATGDLACRFLQLLQASGRGASAHLVVLIALERRRAPGAPLSARAWPGERRCHGIFAPLPRWHLQVYAFYEAVAGFVAPVTVLGVACGHLLSVWWRHRPQAPAAAAPWSASPGRAPAPSALPRAKVQSLKMSLLLALLFVGCELPYFAARLAAAWSSGPAGDWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 3 - DNA, 1993 bp, Homo sapiens
  CDS (116)..(1417)
ctaactttgggaactcgtatagacccagcgtcgctccccgcggtgcctcgcctccactttggtttcccgcgtcctgcccgctctcttcggtgcctcctcttcctccgggacaaggatggaggatctctttagcccctcaattctgccgccggcgcccaacatttccgtgcccatcttgctgggctggggtctcaacctgaccttggggcaaggagcccctgcctctgggccgcccagccgccgcgtccgcctggtgttcctgggggtcatcctggtggtggcggtggcaggcaacaccacagtgctgtgccgcctgtgcggcggcggcgggccctgggcgggccccaagcgtcgcaagatggacttcctgctggtgcagctggccctggcggacctgtacgcgtgcgggggcacggcgctgtcacagctggcctgggaactgctgggcgagccccgcgcggccacgggggacctggcgtgccgcttcctgcagctgctgcaggcatccgggcggggcgcctcggcccacctcgtggtgctcatcgccctcgagcgccggcgcgcggtgcgtcttccgcacggccggccgctgcccgcgcgtgccctcgccgccctgggctggctgctggcactgctgctggcgctgcccccggccttcgtggtgcgcggggactccccctcgccgctgccgccgccgccgccgccaacgtccctgcagccaggcgcgcccccggccgcccgcgcctggccgggggagcgtcgctgccacgggatcttcgcgcccctgccgcgctggcacctgcaggtctacgcgttctacgaggccgtcgcgggcttcgtcgcgcctgttacggtcctgggcgtcgcttgcggccacctactctccgtctggtggcggcaccggccgcaggcccccgcggctgcagcgccctggtcggcgagcccaggtcgagcccctgcgcccagcgcgctgccccgcgccaaggtgcagagcctgaagatgagcctgctgctggcgctgctgttcgtgggctgcgagctgccctactttgccgcccggctggcggccgcgtggtcgtccgggcccgcgggagactgggagggagagggcctgtcggcggcgctgcgcgtggtggcgatggccaacagcgctctcaatcccttcgtctacctcttcttccaggcgggcgactgccggctccggcgacagctgcggaagcggctgggctctctgtgctgcgcgccgcagggaggcgcggaggacgaggaggggccccggggccaccaggcgctctaccgccaacgctggccccaccctcattatcaccatgctcggcgggaaccgctggacgagggcggcttgcgcccaccccctccgcgccccagacccctgccttgctcctgcgaaagtgccttctaggtgcttggtggtcagagacgggtcatctgtcgctaaggcgcaacctccagggaactcgaggcctgccagggtctgtccagatcacaaggggcaggagagtctgtgagagagtgacactgaagttgtccccttcctccactctcctattcccttctcatgtttacatttccctatgctcttccagtttctcttcttccctacagttcctctcatatctccccatttggagacagtgagccactggaaagttgtaaaaacaaaaacagttatttttgcagttttctttcacgcatttatagtgctctggataatgccatttatttttgctgattacccaactttcagtatttgctgtgttatcatctgtatttacttattttgaatcgtgcttaaatcaaatgtaccttcagcacctgcaagtttgccttttctttccaggaggaaaatccccacgttgctctccctggggagtctgagaattataccagtgctgtcagaaatgtaatcatgctgtcatttcagagccacagagtatttataaaataaaaacctttcccacggaaaaaaaaaaaa

SEQ ID NO: 4 - protein, 434 aa, Homo sapiens
MEDLFSPSILPPAPNISVPILLGWGLNLTLGQGAPASGPPSRRVRLVFLGVILVVAVAGNTTVLCRLCGGGGPWAGPKRRKMDFLLVQLALADLYACGGTALSQLAWELLGEPRAATGDLACRFLQLLQASGRGASAHLVVLIALERRRAVRLPHGRPLPARALAALGWLLALLLALPPAFVVRGDSPSPLPPPPPPTSLQPGAPPAARAWPGERRCHGIFAPLPRWHLQVYAFYEAVAGFVAPVTVLGVACGHLLSVWWRHRPQAPAAAAPWSASPGRAPAPSALPRAKVQSLKMSLLLALLFVGCELPYFAARLAAAWSSGPAGDWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 5 - DNA, 20 bp, Homo sapiens
tgcgaaagtgccttctaggt

SEQ ID NO: 6 - DNA, 20 bp, Homo sapiens
cagtggctcactgtctccaa

SEQ ID NO: 7 - DNA, 80 bp, Homo sapiens
ttggtggtcagagacgggtcatctgtcgctaaggcgcaacctccagggaactcgaggcctgccagggtctgtccagatca

SEQ ID NO: 8 - DNA, 20 bp, Homo sapiens
tgcgaaagtgccttctaggt

SEQ ID NO: 9 - DNA, 20 bp, Homo sapiens
cagtggctcactgtctccaa

SEQ ID NO: 10 - DNA, 80 bp, Homo sapiens
tgagagagtgacactgaagttgtccccttcctccactctcctattcccttctcatgtttacatttccctatgctcttcca

SEQ ID NO: 11 - DNA, 18 bp, Bacteriophage T7
atttaggtgacactatag

SEQ ID NO: 12 - DNA, 20 bp, Bacteriophage SP6
taatacgactcactataggg

SEQ ID NO: 13 - protein, 388 aa, Mus musculus
MEGTPAANWSIELDLGSGVPPGAEGNLTAGPPRRNEALARVEVAVLCLILFLALSGNACVLLALRTTRHKHSRLFFFMKHLSIADLVVAVFQVLPQLLWDITFRFYGPDLLCRLVKYLQVVGMFASTYLLLLMSLDRCLAICQPLRSLRRRTDRLAVLATWLGCLVASVPQVHIFSLREVADGVFDCWAVFIQPWGPKAYVTWITLAVYIVPVIVLAACYGLISFKIWQNLRLKTAAAAAAAEGSDAAGGAGRAALARVSSVKLISKAKIRTVKMTFIIVLAFIVCWTPFFFVQMWSVWDVNAPKEASAFIIAMLLASLNSCCNPWIYMLFTGHLFHELVQRFLCCSARYLKGSRPGETSISKKSNSSTFVLSRCSSSQRSCSQPSSA

SEQ ID NO: 14 - protein, 275 aa, Artificial Sequence
  Concensus Pfam Sequence.
GNILVIWVICRHKRMRTPTNYFICNLAVADLLFCLTCPPWMLYYFHWGHHHWPFGRAMCKIWTYFFYMCCYASIFFLCCISIDRYWAICHPMRYRRRMTRPRHAWVMCLVIWVLAFLWSLPPLMFWWCYTHECPNHWNNCNHTWCFIDWPHESWHHWWTWWRYYYICSCIVGFYIPLLVMCFCYCRIYRTLWRHARQMASKMRSRKERKAAKMLCVVVVVFFVCWLPYHIFMFMDTFCMHWWMCWTCELECVIPWAYQICVWLAYVNCCLNPIIY

SEQ ID NO: 15 - DNA, 23 bp, Homo sapiens
caggtgcagctggtgcagtctgg

SEQ ID NO: 16 - DNA, 23 bp, Homo sapiens
caggtcaacttaagggagtctgg

SEQ ID NO: 17 - DNA, 23 bp, Homo sapiens
gaggtgcagctggtggagtctgg

SEQ ID NO: 18 - DNA, 23 bp, Homo sapiens
caggtgcagctgcaggagtcggg

SEQ ID NO: 19 - DNA, 23 bp, Homo sapiens
gaggtgcagctgttgcagtctgc

SEQ ID NO: 20 - DNA, 23 bp, Homo sapiens
caggtacagctgcagcagtcagg

SEQ ID NO: 21 - DNA, 24 bp, Homo sapiens
tgaggagacggtgaccagggtgcc

SEQ ID NO: 22 - DNA, 24 bp, Homo sapiens
tgaagagacggtgaccattgtccc

SEQ ID NO: 23 - DNA, 24 bp, Homo sapiens
tgaggagacggtgaccagggttcc

SEQ ID NO: 24 - DNA, 24 bp, Homo sapiens
tgaggagacggtgaccgtggtccc

SEQ ID NO: 25 - DNA, 23 bp, Homo sapiens
gacatccagatgacccagtctcc

SEQ ID NO: 26 - DNA, 23 bp, Homo sapiens
gatgttgtgatgactcagtctcc

SEQ ID NO: 27 - DNA, 23 bp, Homo sapiens
gatattgtgatgactcagtctcc

SEQ ID NO: 28 - DNA, 23 bp, Homo sapiens
gaaattgtgttgacgcagtctcc

SEQ ID NO: 29 - DNA, 23 bp, Homo sapiens
gacatcgtgatgacccagtctcc

SEQ ID NO: 30 - DNA, 23 bp, Homo sapiens
gaaacgacactcacgcagtctcc

SEQ ID NO: 31 - DNA, 23 bp, Homo sapiens
gaaattgtgctgactcagtctcc

SEQ ID NO: 32 - DNA, 23 bp, Homo sapiens
cagtctgtgttgacgcagccgcc

SEQ ID NO: 33 - DNA, 23 bp, Homo sapiens
cagtctgccctgactcagcctgc

SEQ ID NO: 34 - DNA, 23 bp, Homo sapiens
tcctatgtgctgactcagccacc

SEQ ID NO: 35 - DNA, 23 bp, Homo sapiens
tcttctgagctgactcaggaccc

SEQ ID NO: 36 - DNA, 23 bp, Homo sapiens
cacgttatactgactcaaccgcc

SEQ ID NO: 37 - DNA, 23 bp, Homo sapiens
caggctgtgctcactcagccgtc

SEQ ID NO: 38 - DNA, 23 bp, Homo sapiens
aattttatgctgactcagcccca

SEQ ID NO: 39 - DNA, 24 bp, Homo sapiens
acgtttgatttccaccttggtccc

SEQ ID NO: 40 - DNA, 24 bp, Homo sapiens
acgtttgatctccagcttggtccc

SEQ ID NO: 41 - DNA, 24 bp, Homo sapiens
acgtttgatatccactttggtccc

SEQ ID NO: 42 - DNA, 24 bp, Homo sapiens
acgtttgatctccaccttggtccc

SEQ ID NO: 43 - DNA, 24 bp, Homo sapiens
acgtttaatctccagtcgtgtccc

SEQ ID NO: 44 - DNA, 23 bp, Homo sapiens
cagtctgtgttgacgcagccgcc

SEQ ID NO: 45 - DNA, 23 bp, Homo sapiens
cagtctgccctgactcagcctgc

SEQ ID NO: 46 - DNA, 23 bp, Homo sapiens
tcctatgtgctgactcagccacc

SEQ ID NO: 47 - DNA, 23 bp, Homo sapiens
tcttctgagctgactcaggaccc

SEQ ID NO: 48 - DNA, 23 bp, Homo sapiens
cacgttatactgactcaaccgcc

SEQ ID NO: 49 - DNA, 23 bp, Homo sapiens
caggctgtgctcactcagccgtc

SEQ ID NO: 50 - DNA, 23 bp, Homo sapiens
aattttatgctgactcagcccca

SEQ ID NO: 51 - DNA, 19 bp, Homo sapiens
ccttgctcctgcgaaagtg

SEQ ID NO: 52 - DNA, 19 bp, Homo sapiens
gcgccttagcgacagatga

SEQ ID NO: 53 - DNA, 28 bp, Homo sapiens
ccgtctctgaccaccaagcacctagaag

SEQ ID NO: 54 - protein, 118 aa, Homo sapiens
AAAWSSGPAGDWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 55 - protein, 108 aa, Homo sapiens
DWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 56 - protein, 110 aa, Homo sapiens
AGDWEGEGLSAALRVVAMANSALNPFVYLFFQAGDCRLRRQLRKRLGSLCCAPQGGAEDEEGPRGHQALYRQRWPHPHYHHARREPLDEGGLRPPPPRPRPLPCSCESAF

SEQ ID NO: 57 - protein, 69 aa, Homo sapiens
MANSALNPFVYLFFQAGDCRLRRQLRKRLGSLCCAPQGGAEDEEGPRGHQALYRQRWPHPHYHHARREP

SEQ ID NO: 58 - protein, 25 aa, Homo sapiens
PLDEGGLRPPPPRPRPLPCSCESAF

SEQ ID NO: 59 - protein, 81 aa, Homo sapiens
AAAWSSGPAGDWEGEGLSAALRVVAMANSALNPFVYLFFQAGDCRLRRQLRKRLGSLCCAPQGGAEDEEGPRGHQALYRQR

SEQ ID NO: 60 - protein, 36 aa, Homo sapiens
PHPHYHHARREPLDEGGLRPPPPRPRPLPCSCESAF

SEQ ID NO: 61 - protein, 67 aa, Homo sapiens
MEDLFSPSILPPAPNISVPILLGWGLNLTLGQGAPASGPPSRRVRLVFLGVILVVAVAGNTTVLCRL

SEQ ID NO: 62 - protein, 47 aa, Homo sapiens
EGLSAALRVVAMANSALNPFVYLFFQAGDCRLRRQLRKRLGSLCCAP

SEQ ID NO: 63 - protein, 21 aa, Homo sapiens
VAMANSALNPFVYLFFQAGDC

(19) United States
(12) Patent Application Publication
(10) Pub. No.: US 2004/0086881 A1
(43) Pub. Date: May 6, 2004

(54) Novel human G-protein coupled receptor, BMSOTR, and splice variant thereof

(76) Inventors: Chandra S. Ramanathan, Wallingford, CT (US); Shuba Gopal, New York, NY (US); Gabriel A. Mintier, Hightstown, NJ (US); John Feder, Belle Mead, NJ (US)

Correspondence Address: STEPHEN B. DAVIS, BRISTOL-MYERS SQUIBB COMPANY, PATENT DEPARTMENT, P O BOX 4000, PRINCETON, NJ, 08543-4000, US

(21) Appl. No.: 10/334,360

(22) Filed: Mar. 12, 2003

Related U.S. Application Data
(60) Provisional application No. 60/345,706, filed on Jan. 4, 2002.
(60) Provisional application No. 60/355,559, filed on Feb. 6, 2002.

(51) Int. Cl. C12Q 1/68; C07H 21/04; C07K 14/705; C07K 16/28

(52) U.S. Cl. 435/6; 435/69.1; 435/320.1; 435/325; 530/350; 536/23.5; 530/388.22

(57) ABSTRACT

The present invention describes the novel human G-protein coupled receptor (GPCR) BMSOTR and its encoding polynucleotide. Also described are expression vectors, host cells, antisense molecules, and antibodies associated with the BMSOTR polynucleotide and/or polypeptide of this invention. In addition, methods for treating, diagnosing, preventing, and screening for disorders or diseases associated with abnormal biological activity of BMSOTR are described, as are methods for screening for modulators, e.g., agonists or antagonists, of BMSOTR activity and/or function.

This application claims benefit to provisional application U.S. Serial No. 60/345,706 filed Jan. 4, 2002; and to provisional application U.S. Serial No. 60/355,559, filed Feb. 6, 2002. The entire teachings of the referenced applications are incorporated herein by reference.

### FIELD OF THE INVENTION

The present invention relates to a novel human G-protein coupled receptor (GPCR), called BMSOTR herein, nucleic acid or polynucleotide sequence which encodes a BMSOTR protein. The BMSOTR protein is a candidate member of the oxytocin receptor protein family. This invention further relates to fragments of the BMSOTR nucleic acid sequence and its encoded amino acid sequence. Additionally, the invention relates to methods of using the BMSOTR polynucleotide sequence and encoded BMSOTR protein for diagnosis, genetic screening and for the treatment of diseases, disorders, conditions, or syndromes associated with BMSOTR.

### BACKGROUND OF THE INVENTION

Many medically significant biological processes that are mediated by proteins participating in signal transduction pathways involving G-proteins and/or second messengers, e.g., cAMP, have been established (Lefkowitz, Nature, 351:353-354 (1991)). These proteins are referred to herein as proteins participating in pathways with G-proteins or PPG proteins. Some examples of these proteins include the G protein-coupled receptors (GPCR), such as those for adrenergic agents and dopamine (Kobilka, B. K., et al., PNAS, 84:46-50 (1987); Kobilka, B. K., et al., Science, 238:650-656 (1987); Bunzow, J. R., et al., Nature, 336:783-787 (1988)), G-proteins themselves, effector proteins, e.g., phospholipase C, adenylate cyclase, and phosphodiesterase, and actuator proteins, e.g., protein kinase A and protein kinase C (Simon, M. I., et al., Science, 252:802-8 (1991)).

For example, in one form of signal transduction, the effect of hormone binding results in activation of the enzyme adenylate cyclase inside the cell. Enzyme activation by hormones is dependent on the presence of the nucleotide GTP, as GTP influences hormone binding. A G-protein binds the hormone receptors to adenylate cyclase. The G-protein has further been shown to exchange GTP for bound GDP when activated by hormone receptors. The GTP-carrying form then binds to an activated adenylate cyclase. Hydrolysis of GTP to GDP, catalyzed by the G-protein itself, returns the G-protein to its basal, inactive form. Thus, the G-protein serves a dual role—as an intermediate that relays the signal from receptor to effector, and as a “clock” that controls the duration of the signal.

The membrane protein gene superfamily of G-protein coupled receptors (GPCRs) has been characterized as having seven putative transmembrane domains. The domains are believed to represent transmembrane α-helices connected by extracellular or cytoplasmic loops. GPCRs include a wide range of biologically active receptors, such as hormone, viral, growth factor, and neuronal receptors.

GPCRs are further characterized as having seven conserved hydrophobic stretches of about 20 to 30 amino acids, connecting at least eight divergent hydrophilic loops. The G-protein family of coupled receptors includes dopamine receptors, which bind to neuroleptic drugs, used for treating psychotic and neurological disorders. Other examples of members of this family of receptors include calcitonin, adrenergic, endothelin, cAMP, adenosine, muscarinic, acetylcholine, serotonin, histamine, thrombin, kinin, follicle stimulating hormone, opsins, endothelial differentiation gene-1 receptor, rhodopsins, odorant and cytomegalovirus receptors, etc.

Most GPCRs have single conserved cysteine residues in each of the first two extracellular loops which form disulfide bonds that are believed to stabilize functional protein structure. The 7 transmembrane regions are designated as TM1, TM2, TM3, TM4, TM5, TM6, and TM7. TM3 has been implicated in signal transduction.

Phosphorylation and lipidation (palmitylation or famesylation) of cysteine residues can influence signal transduction of some GPCRs. Most GPCRs contain potential phosphorylation sites within the third cytoplasmic loop and/or the carboxyl terminus. For several GPCRs, such as the β-adrenoreceptor, phosphorylation by protein kinase A and/or specific receptor kinases mediates receptor desensitization.

For some receptors, the ligand binding sites of GPCRs are believed to comprise a hydrophilic socket formed by the transmembrane domains of several GPCRs. This socket is surrounded by hydrophobic residues of the GPCRs. The hydrophilic side of each GPCR transmembrane helix is postulated to face inward and form the polar ligand-binding site. TM3 has been implicated in several GPCRs as having a ligand-binding site, which includes the TM3 aspartate residue. Additionally, TM5 serines, a TM6 asparagine and TM6 or TM7 phenylalanines or tyrosines are also implicated in ligand binding.

GPCRs can be intracellularly coupled by heterotrimeric G-proteins to various intracellular enzymes, ion channels and transporters (see, Johnson et al., Endoc., Rev., 10:317-331(1989)). Different G-protein β-subunits preferentially stimulate particular effectors to modulate various biological functions in a cell. Phosphorylation of cytoplasmic residues of GPCRs have been identified as an important mechanism for the regulation of G-protein coupling of some GPCRs. GPCRs are found in numerous sites within a mammalian host.

GPCRs are one of the largest receptor superfamilies known. These receptors are biologically important and malfunction of these receptors results in diseases such as Alzheimer's, Parkinson, diabetes, dwarfism, color blindness, retinal pigmentosa and asthma. GPCRs are also involved in depression, schizophrenia, sleeplessness, hypertension, anxiety, stress, renal failure and in several other cardiovascular, metabolic, neural, oncology and immune disorders (F. Horn and G. Vriend, J. Mol. Med., 76: 464-468 (1998)). They have also been shown to play a role in HIV infection (Y. Feng et al., Science, 272: 872-877 (1996)).

The structure of GPCRs consists of seven transmembrane helices that are connected by loops. The N-terminus is always extracellular and C-terminus is intracellular. GPCRs are involved in signal transduction. The signal is received at the extracellular N-terminus side. The signal can be an endogenous ligand, a chemical moiety or light. This signal is then transduced through the membrane to the cytosolic side where a heterotrimeric protein G-protein is activated which in turn elicits a response (F. Horn et al., Recept. and Chann., 5: 305-314 (1998)). Ligands, agonists and antagonists, for these GPCRs are used for therapeutic purposes.

The oxytocin receptor (OTR) belongs to the superfamily of GPCRs. To date, only a single OTR has been cloned from any individual species. Low stringency hybridization studies of human genomic DNA with human OTR cDNA revealed the existence of only one OTR (Kimura, T. et al. Endocrine J., 42: 607-615 (1995)). However, some pharmacological studies have indicated the possible existence of OTR subtypes (El Alj, A. et al. Eur. J. Pharmacol., 186: 231-238 (1990)), (Chan, W. et al. Endocrinology, 132: 1381-1386 (1993)). OTR structures have been identified for the human (Kimura, T. et al., Nature, 356: 526-529 (1992)), pig (Gorbulev, V. et al., Eur. J. Biochem., 215:1-7 (1993)), rat (Rozen, F. et al. Proc. Natl. Acad. Sci. USA, 92:200-204 (1995)), sheep (Riley, P. et al., J. Mol. Endocrinol., 15:195-202 (1995)), cow (Bathgate, R. et al., DNA Cell Biol., 14:1037-1048 (1995)), mouse (Kubota, Y. et al., Mol. Cell. Endocrinol., 124: 25-32 (1996)), and rhesus monkey (Salvatore, C. et al., J. Recept. Signal Transduct. Res., 18: 15-24 (1998)). All of the cloned receptors are classic GPCRs with seven transmembrane-spanning domains and are highly homologous across species.

The OTR binds the neurohypophysial hormone oxytocin. The best-documented responses to oxytocin/OTR are an increase in the frequency and intensity of uterine contraction at parturition and the contraction of myoepithelial cells resulting in milk ejection from the mammary gland (Soloff, M. et al., Science, 204: 1313-1315 (1979)). In addition, studies utilizing reverse-transcription PCR methods have identified the gene encoding uterine OTR in most of the organs previously reported to manifest oxytocin binding, including the mammary gland, pituitary, brain, kidney, thymus, ovary, testes, and heart (Breton, C. et al., Endocrinology, 138: 1857-1862 (1997); Gutkowska, J. et al., Proc. Natl. Acad. Sci. USA, 94:11704-11709 (1997); Zingg. H., Baillieres Clin. Endocrinol. Metab., 10:75-96 (1996)). Consistent with the wide expression range of OTR, studies have implicated the importance of the OTR with the female reproductive system, the male reproductive tract, mammary tissues, kidney function, heart and cardiovascular system, thymic involution, sexual behavior, maternal behavior, social behavior, stress-related behavior, feeding and grooming, memory and learning, tolerance and dependence to opioids, and to such disorders as obsessive-compulsive disorder, anorexia, Prader-Willi syndrome, depression, schizophrenia, Alzheimer's disease and Parkinson's disease (Gimpl, G. et al., Physiological Reviews, 81:629-683 (2001)).

Several studies have suggested the presence of different OTR subtypes in various tissues. In rats, uterus OTR antagonists block oxytocin-induced myometrial contractile activity but, surprisingly, cause agonistic effects on endometrial prostaglandin release, an action previously attributed to oxytocin stimulation of endometrial OTRs (Chan, W. et al, Endocrinology, 132:1381-1386 (1993)). Additional studies showed that the oxytocin agonist [Thr⁴, Gly⁷] oxytocin stimulated myometrial contractions, but, unlike oxytocin, the agonist failed to stimulate endometrial prostaglandin release (Chen, D. et al., J. reprod. Fertil., 102: 337-343 (1994)). These results suggest separate endometrial and myometrial OTR subtypes. Possible subtypes of OTRs have also been suggested in the kidney (Arpin-Bott, M. et al, J. Endocrinol., 153: 49-59 (1997)).

Furthermore, although most brain areas which contain oxytocin-binding sites have been shown to express OTR mRNA using cDNA probes derived from the uterine OTR (Yoshimura, R., Endocrinology, 133: 1239-1246 (1993)), antibodies raised against the uterine OTR were not immunoreactive with the hippocampus and the amygdala (Adan et al., Endocrinology, 136: 4022-4028 (1995)). In the uterus, mammary gland and other areas of the brain, such immunoreactivity was easily detected (Adan et al., Endocrinology, 136: 4022-4028 (1995)). Thus, the possibility of a second OTR or OTR subtypes persists, and would help to explain differential tissue responses to oxytocin antagonists/agonists, as well as why OTR second messenger coupling may vary in different parts of the brain (McCarthy, M. et al., Soc. Neurosci. Abstr., 24:632 (1998)), and the wide-range of physiological and mental effects associated with oxytocin and OTR.

### SUMMARY OF THE INVENTION

The present invention provides a novel member of the human GPCR oxytocin receptor subfamily, BMSOTR, and a BMSOTR variant. Based on sequence homology, the protein BMSOTR has been determined to be a member of the GPCR class of proteins. In particular, BMSOTR of this invention is most similar to the oxytocin receptor (OTR) protein family.

The present invention provides the BMSOTR polynucleotide, preferably full-length, and its encoded polypeptide. The BMSOTR polynucleotide and polypeptide, may be involved in a variety of diseases, disorders and conditions associated with BMSOTR activity, which include, but are not limited to, immune-related disorders, acute heart failure, hypotension, hypertension, endocrinal diseases, growth disorders, neuropathic pain, obesity, anorexia, HIV infections, cancers, bulimia, asthma, Parkinson's disease, osteoporosis, angina pectoris, myocardial infarction, psychotic, metabolic, cardiovascular and neurological disorders.

More specifically, the present invention is concerned with modulation of the BMSOTR polynucleotide and encoded products, particularly in providing treatments and therapies for relevant diseases. Antagonizing or inhibiting the action of the BMSOTR polynucleotide and polypeptide is especially encompassed by the present invention. The high similarity between BMSOTR and OTR implicates BMSOTR in oxytocin-related systems and conditions, such as the female reproductive system, the male reproductive tract, mammary tissues, kidney function, heart and cardiovascular system, thymic involution, sexual behavior, maternal behavior, social behavior, stress-related behavior, feeding and grooming, memory and learning, tolerance and dependence to opioids, and to such disorders as obsessive-compulsive disorder, anorexia, Prader-Willi syndrome, depression, schizophrenia, Alzheimer's disease, and Parkinson's disease.

It is another object of this invention to provide the isolated BMSOTR polynucleotide as depicted in SEQ ID NO:1. Also provided is the BMSOTR polypeptide, encoded by the polynucleotide of SEQ ID NO:1 and having the encoded amino acid sequence of SEQ ID NO:2, or a functional or biologically active portion of this sequence. The BMSOTR polypeptide is of the GPCR type, comprising several types of GPCRs, namely, sensory GPCRs, orphan GPCRs, chemokine GPCRs, or very large GPCRs. GPCRs have been described in relation to dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-1 receptors, odorant receptors, taste receptors, and cytomegalovirus receptors.

It is yet another object of the invention to provide the isolated BMSOTR variant polynucleotide as depicted in SEQ ID NO:3. Also provided is the BMSOTR variant polypeptide, encoded by the polynucleotide of SEQ ID NO:3 and having the encoded amino acid sequence of SEQ ID NO:4, or a functional or biologically active portion of this sequence.

Another object of the invention to provide compositions comprising the BMSOTR polynucleotide sequence, or fragments or portions thereof, or the encoded BMSOTR polypeptide, or fragments or portions thereof. In addition, this invention provides pharmaceutical compositions comprising at least one BMSOTR polypeptide, or functional portion thereof, wherein the compositions further comprise a pharmaceutically and/or physiologically acceptable carrier, excipient, or diluent.

Yet another object of the invention is to provide compositions comprising N-terminal, C-terminal or internal deletion polypeptides of the encoded BMSOTR polypeptide. Polynucleotides encoding these deletion polypeptides are also provided. The present invention also provides the use of these polypeptides as an immunogenic and/or antigenic epitope as described elsewhere herein.

A further object of this invention is to provide the polynucleotide sequence comprising the complement of SEQ ID NO:1, or variants thereof. In addition, an object of the invention encompasses variations or modifications of the BMSOTR sequence which are the result of degeneracy of the genetic code, where the polynucleotide sequences can hybridize under moderate or high stringency conditions to the polynucleotide sequence of SEQ ID NO:1.

Another object of the invention is to provide the polynucleotide sequence of BMSOTR (SEQ ID NO:1) lacking the initiating codon as well as the resulting encoded polypeptide. Specifically, the present invention provides the polynucleotide corresponding to nucleotides 119 through 1264 of SEQ ID NO:1, and the polypeptide corresponding to amino acids 2 through 383 of SEQ ID NO:2. Also provided by the present invention are recombinant vectors comprising said encoding sequence, and host cells comprising said vector.

Another object of the invention is to provide the polynucleotide sequence of BMSOTR variant (SEQ ID NO:3) lacking the initiating codon as well as the resulting encoded polypeptide. Specifically, the present invention provides the polynucleotide corresponding to nucleotides 119 through 1417 of SEQ ID NO:3, and the polypeptide corresponding to amino acids 2 through 434 of SEQ ID NO:4. Also provided by the present invention are recombinant vectors comprising said encoding sequence, and host cells comprising said vector.

It is another object of the invention to provide an antisense of the BMSOTR nucleic acid sequence, as well as oligonucleotides, fragments, or portions of the nucleic acid molecules or antisense molecules. Also provided are expression vectors and host cells comprising polynucleotides that encode the BMSOTR polypeptide.

In yet another of its objects, the present invention provides pharmaceutical compositions comprising the BMSOTR polynucleotide sequence, or fragments thereof, or the encoded BMSOTR polypeptide sequence, or fragments or portions thereof. Also provided are pharmaceutical compositions comprising the BMSOTR polypeptide sequence, homologues, or one or more functional portions thereof, wherein the compositions further comprise a pharmaceutically- and/or physiologically-acceptable carrier, excipient, or diluent. All fragments or portions of the BMSOTR polynucleotide and polypeptide are preferably functional or active.

Another object of the invention is to provide methods for producing a polypeptide comprising the amino acid sequence of SEQ ID NO:2, or a fragment thereof, preferably, a functional fragment or portion thereof, comprising the steps of a) cultivating a host cell containing an expression vector containing at least a functional fragment of the polynucleotide sequence encoding the BMSOTR protein according to this invention, under conditions suitable for the expression of the polypeptide; and b) recovering the polypeptide from the host cell or lysate thereof.

Another object of this invention is to provide a substantially purified modulator, preferably an antagonist or inhibitor, of the BMSOTR polypeptide having SEQ ID NO:2. In this regard, and by way of example, a purified antibody, or binding portion thereof that binds to a polypeptide comprising the amino acid sequence of SEQ ID NO:2 or antigenic epitope thereof, or homologue encoded by a polynucleotide having homology to the nucleic acid sequence, or degenerate thereof, as set forth in SEQ ID NO:1 is provided.

It is yet another object of the present invention to provide BMSOTR nucleic acid sequences, polypeptides, peptides and antibodies for use in the diagnosis and/or screening of disorders or diseases associated with expression of the BMSOTR polynucleotide and its encoded polypeptide product as described herein.

Another object of this invention is to provide diagnostic probes or primers for detecting BMSOTR-related diseases and/or for monitoring a patient's response to therapy. The probe or primer sequences comprise nucleic acid or amino acid sequences of BMSOTR described herein.

It is another object of the present invention to provide a method for detecting a polynucleotide that encodes the BMSOTR polypeptide in a biological sample comprising the steps of: a) hybridizing the complement of the polynucleotide sequence encoding SEQ ID NO:1 or a hybridizable portion thereof, to the nucleic acid material of a biological sample, thereby forming a hybridization complex; and b) detecting the hybridization complex, wherein the presence of the complex correlates with the presence of a polynucleotide encoding a BMSOTR polypeptide in the biological sample. The nucleic acid material may be further amplified by the polymerase chain reaction (PCR) prior to hybridization, as known and practiced in the art.

Another object of this invention is to provide methods for screening for agents which modulate the BMSOTR polypeptide, e.g., agonists (or enhancers or activators) and antagonists (or blockers or inhibitors), particularly those that are obtained from the screening methods as described.

As yet a further object, the present invention provides methods for detecting genetic predisposition, susceptibility and/or response to therapy of various BMSOTR-related diseases, disorders, or conditions.

It is another object of the present invention to provide methods for the treatment or prevention of several BMSOTR-associated diseases or disorders including, but not limited to, reproductive-system disorders, endocrine-system disorders, neurological and behavioral disorders. The methods involve administering to an individual in need of such treatment or prevention an effective amount of a modulator of the BMSOTR polypeptide. Preferred are BMSOTR antagonists. As a result of its high similarity to the oxytocin receptor, the BMSOTR molecule may be involved in oxytocin-related disorders, requiring antagonism of its activity.

It is yet another object of this invention to provide diagnostic kits for the determination of the nucleotide sequences of human BMSOTR alleles. The kits comprise reagents and instructions for amplification-based assays, nucleic acid probe assays, protein nucleic acid probe assays, antibody assays or any combination thereof. Such kits are suitable for screening and for the diagnosis of disorders associated with aberrant or uncontrolled cellular proliferation or development, and with the expression of BMSOTR polynucleotide and encoded BMSOTR polypeptide in a sample, as described herein.

The above-mentioned objects of the invention are also provided for the BMSOTR variant polynucleotide (SEQ ID NO:3) and its encoded polypeptide (SEQ ID NO:4).

Further objects, features, and advantages of the present invention will be better understood upon a reading of the detailed description of the invention when considered in connection with the accompanying figures or drawings.

The invention further relates to a polynucleotide encoding a polypeptide fragment of SEQ ID NO:2 and/or 4, or a polypeptide fragment encoded by the cDNA sequence included in the deposited clone, which is hybridizable to SEQ ID NO:1 and/or 3.

The invention further relates to a polynucleotide encoding a polypeptide domain of SEQ ID NO:2 and/or 4 or a polypeptide domain encoded by the cDNA sequence included in the deposited clone, which is hybridizable to SEQ ID NO:1 and/or 3.

The invention further relates to a polynucleotide encoding a polypeptide epitope of SEQ ID NO:2 and/or 4 or a polypeptide epitope encoded by the cDNA sequence included in the deposited clone, which is hybridizable to SEQ ID NO:1 and/or 3.

The invention further relates to a polynucleotide encoding a polypeptide of SEQ ID NO:2 and/or 4 or the cDNA sequence included in the deposited clone, which is hybridizable to SEQ ID NO:1 and/or 3, having biological activity.

The invention further relates to a polynucleotide which is a variant of SEQ ID NO:1 and/or 3.

The invention further relates to a polynucleotide which is an allelic variant of SEQ ID NO:1 and/or 3.

The invention further relates to a polynucleotide which encodes a species homologue of the SEQ ID NO:2 and/or 4.

The invention further relates to a polynucleotide which represents the complimentary sequence (antisense) of SEQ ID NO:1 and/or 3.

The invention further relates to a polynucleotide capable of hybridizing under stringent conditions to any one of the polynucleotides specified herein, wherein said polynucleotide does not hybridize under stringent conditions to a nucleic acid molecule having a nucleotide sequence of only A residues or of only T residues.

The invention further relates to an isolated nucleic acid molecule of SEQ ID NO:2 and/or 4, wherein the polynucleotide fragment comprises a nucleotide sequence encoding an BMSOTR and/or BMSOTR variant protein.

The invention further relates to an isolated nucleic acid molecule of SEQ ID NO:1 and/or 3, wherein the polynucleotide fragment comprises a nucleotide sequence encoding the sequence identified as SEQ ID NO:2 and/or 4 or the polypeptide encoded by the cDNA sequence included in the deposited clone, which is hybridizable to SEQ ID NO:1 and/or 3.

The invention further relates to an isolated nucleic acid molecule of of SEQ ID NO:1 and/or 3, wherein the polynucleotide fragment comprises the entire nucleotide sequence of SEQ ID NO:1 and/or 3 or the cDNA sequence included in the deposited clone, which is hybridizable to SEQ ID NO:1 and/or 3.

The invention further relates to an isolated nucleic acid molecule of SEQ ID NO:1 and/or 3, wherein the nucleotide sequence comprises sequential nucleotide deletions from either the C-terminus or the N-terminus.

The invention further relates to an isolated polypeptide comprising an amino acid sequence that comprises a polypeptide fragment of SEQ ID NO:2 and/or 4 or the encoded sequence included in the deposited clone.

The invention further relates to a polypeptide fragment of SEQ ID NO:2 and/or 4 or the encoded sequence included in the deposited clone, having biological activity.

The invention further relates to a polypeptide domain of SEQ ID NO:2 and/or 4 or the encoded sequence included in the deposited clone.

The invention further relates to a polypeptide epitope of SEQ ID NO:2 and/or 4 or the encoded sequence included in the deposited clone.

The invention further relates to a full length protein of SEQ ID NO:2 and/or 4 or the encoded sequence included in the deposited clone.

The invention further relates to a variant of SEQ ID NO:2 and/or 4.

The invention further relates to an allelic variant of SEQ ID NO:2 and/or 4.

The invention further relates to a species homologue of SEQ ID NO:2 and/or 4.

The invention further relates to the isolated polypeptide of of SEQ ID NO:2 and/or 4, wherein the full length protein comprises sequential amino acid deletions from either the C-terminus or the N-terminus.

The invention further relates to an isolated antibody that binds specifically to the isolated polypeptide of SEQ ID NO:2 and/or 4.

The invention further relates to a method for preventing, treating, or ameliorating a medical condition, comprising administering to a mammalian subject a therapeutically effective amount of the polypeptide of SEQ ID NO:2 and/or 4 or the polynucleotide of SEQ ID NO:1 and/or 3.

The invention further relates to a method of diagnosing a pathological condition or a susceptibility to a pathological condition in a subject comprising the steps of (a) determining the presence or absence of a mutation in the polynucleotide of SEQ ID NO:1 and/or 3; and (b) diagnosing a pathological condition or a susceptibility to a pathological condition based on the presence or absence of said mutation.

The invention further relates to a method of diagnosing a pathological condition or a susceptibility to a pathological condition in a subject comprising the steps of (a) determining the presence or amount of expression of the polypeptide of of SEQ ID NO:2 and/or 4 in a biological sample; and diagnosing a pathological condition or a susceptibility to a pathological condition based on the presence or amount of expression of the polypeptide.

The invention further relates to a method for identifying a binding partner to the polypeptide of SEQ ID NO:2 and/or 4 comprising the steps of (a) contacting the polypeptide of SEQ ID NO:2 and/or 4 with a binding partner; and (b) determining whether the binding partner effects an activity of the polypeptide.

The invention further relates to a gene corresponding to the cDNA sequence of SEQ ID NO:1 and/or 3.

The invention further relates to a method of identifying an activity in a biological assay, wherein the method comprises the steps of expressing SEQ ID NO:1 and/or 3 in a cell, (b) isolating the supernatant; (c) detecting an activity in a biological assay; and (d) identifying the protein in the supernatant having the activity.

The invention further relates to a process for making polynucleotide sequences encoding gene products having altered SEQ ID NO:2 and/or 4 activity comprising the steps of (a) shuffling a nucleotide sequence of SEQ ID NO:1 and/or 3, (b) expressing the resulting shuffled nucleotide sequences and, (c) selecting for altered activity as compared to the activity of the gene product of said unmodified nucleotide sequence.

The invention further relates to a shuffled polynucleotide sequence produced by a shuffling process, wherein said shuffled DNA molecule encodes a gene product having enhanced tolerance to an inhibitor of SEQ ID NO:2 and/or 4 activity.

The invention further relates to a method for preventing, treating, or ameliorating a medical condition with the polypeptide provided as SEQ ID NO:2 and/or 4, in addition to, its encoding nucleic acid, wherein the medical condition is a neural disorder.

The invention further relates to a method for preventing, treating, or ameliorating a medical condition with the polypeptide provided as SEQ ID NO:2 and/or 4, in addition to, its encoding nucleic acid, wherein the medical condition is a disorder related to aberrant signal transduction.

The invention further relates to a method for preventing, treating, or ameliorating a medical condition with the polypeptide provided as SEQ ID NO:2 and/or 4, in addition to, its encoding nucleic acid, wherein the medical condition is a metabolic disorder.

The invention further relates to a method for preventing, treating, or ameliorating a medical condition with the polypeptide provided as SEQ ID NO:2 and/or 4, in addition to, its encoding nucleic acid, wherein the medical condition is a pancreatic disorder.

The invention further relates to a method for preventing, treating, or ameliorating a medical condition with the polypeptide provided as SEQ ID NO:2 and/or 4, in addition to, its encoding nucleic acid, or a modulator thereof, wherein the medical condition is ovarian cancer or related proliferative condition of the ovary.

The invention further relates to a method of diagnosing a pathological condition or a susceptibility to a pathological condition in a subject comprising the steps of (a) determining the presence or amount of expression of the polypeptide of of SEQ ID NO:2 and/or 4 in a biological sample; (b) and diagnosing a pathological condition or a susceptibility to a pathological condition based on the presence or amount of expression of the polypeptide relative to a control, wherein said condition is a member of the group consisting of ovarian cancer, colon cancer, leukemias, and melanoma.

The present invention also relates to an isolated polynucleotide consisting of a portion of the human BMSOTR and/or BMSOTR variant gene consisting of at least 8 bases, specifically excluding Genbank Accession Nos. AI140809; AW592243; AI051313; BE044200; AI187401; AI768383; AI798227; BI752897; AI470444; and/or AA845448.

The present invention also relates to an isolated polynucleotide consisting of a nucleotide sequence encoding a fragment of the human BMSOTR and/or BMSOTR variant protein, wherein said fragment displays one or more functional activities specifically excluding Genbank Accession Nos. AI40809; AW592243; AI051313; BE044200; AI187401; AI768383; AI798227; BI752897; AI470444; and/or AA845448.

The present invention also relates to the polynucleotide of SEQ ID NO:1 and/or 3 consisting of at least 10 to 50 bases, wherein said at least 10 to 50 bases specifically exclude the polynucleotide sequence of Genbank Accession Nos. AI140809; AW592243; AI051313; BE044200; AI187401; AI768383; AI798227; B1752897; AI470444; and/or AA845448.

The present invention also relates to the polynucleotide of SEQ ID NO:1 and/or 3 consisting of at least 15 to 100 bases, wherein said at least 15 to 100 bases specifically exclude the polynucleotide sequence of Genbank Accession Nos. AI140809; AW592243; AI051313; BE044200; AI187401; AI768383; AI798227; B1752897; AI470444; and/or AA845448.

The present invention also relates to the polynucleotide of SEQ ID NO:1 and/or 3 consisting of at least 100 to 1000 bases, wherein said at least 100 to 1000 bases specifically exclude the polynucleotide sequence of Genbank Accession Nos. AI140809; AW592243; AI051313; BE044200; AI187401; AI768383; AI798227; B1752897; AI470444; and/or AA845448.

The present invention also relates to an isolated polypeptide fragment of the human BMSOTR and/or BMSOTR variant protein, wherein said polypeptide fragment does not consist of the polypeptide encoded by the polynucleotide sequence of Genbank Accession Nos. AI140809; AW592243; AI051313; BE044200; AI187401; AI768383; AI798227; BI752897; AI470444; and/or AA845448.

The invention further relates to a method of identifying a compound that modulates the biological activity of BMSOTR and/or BMSOTR variant, comprising the steps of, (a) combining a candidate modulator compound with BMSOTR and/or BMSOTR variant having the sequence set forth in one or more of SEQ ID NO:2; and measuring an effect of the candidate modulator compound on the activity of BMSOTR and/or BMSOTR variant.

The invention further relates to a method of identifying a compound that modulates the biological activity of a GPCR, comprising the steps of, (a) combining a candidate modulator compound with a host cell expressing BMSOTR and/or BMSOTR variant having the sequence as set forth in SEQ ID NO:2; and, (b) measuring an effect of the candidate modulator compound on the activity of the expressed BMSOTR and/or BMSOTR variant.

The invention further relates to a method of identifying a compound that modulates the biological activity of BMSOTR and/or BMSOTR variant, comprising the steps of, (a) combining a candidate modulator compound with a host cell containing a vector described herein, wherein BMSOTR and/or BMSOTR variant is expressed by the cell; and, (b) measuring an effect of the candidate modulator compound on the activity of the expressed BMSOTR and/or BMSOTR variant.

The invention further relates to a method of screening for a compound that is capable of modulating the biological activity of BMSOTR and/or BMSOTR variant, comprising the steps of: (a) providing a host cell described herein; (b) determining the biological activity of BMSOTR and/or BMSOTR variant in the absence of a modulator compound; (c) contacting the cell with the modulator compound; and (d) determining the biological activity of BMSOTR and/or BMSOTR variant in the presence of the modulator compound; wherein a difference between the activity of BMSOTR and/or BMSOTR variant in the presence of the modulator compound and in the absence of the modulator compound indicates a modulating effect of the compound.

The invention further relates to a compound that modulates the biological activity of human BMSOTR and/or BMSOTR variant as identified by the methods described herein.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are HEK cells.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, and further wherein said cells express the polypeptide at either low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said candidate compound is a small molecule, a peptide, or an antisense molecule, wherein said candidate compound is an agonist or antagonist.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are CHO cells that comprise a vector comprising the coding sequence of the beta lactamase gene under the control of NFAT response elements, wherein said cells further comprise a vector comprising the coding sequence of G alpha 15 under conditions wherein G alpha 15 is expressed, wherein said cells express beta lactamase at low, moderate, or high levels.

The invention further relates to a method of screening for candidate compounds capable of modulating the activity of a G-protein coupled receptor polypeptide, comprising: (i) contacting a test compound with a cell or tissue comprising an expression vector capable of expressing a polypeptide comprising an amino acid sequence as set forth in SEQ ID NO:2 or 4, or encoded by ATCC deposit BMSOTR and/or BMSOTR variant, under conditions in which said polypeptide is expressed; and (ii) selecting as candidate modulating compounds those test compounds that modulate activity of the G-protein coupled receptor polypeptide, wherein said cells are HEK cells wherein said cells comprise a vector comprising the coding sequence of the beta lactamase gene under the control of CRE response elements, wherein said cells express beta lactamase at low, moderate, or high levels.

The statement, “wherein said cells express beta lactamase at low, moderate, or high levels” is a reference to cells that either express beta lactamase at low, moderate, or high levels relative to the expression levels of a reference mRNA, gene, or protein; or a reference to the actual percentage of cells that express beta lactamase. In the latter example, high levels of expression would be achieved if the majority of cells were expressing beta lactamase, while low levels of expression would be achieved if only a subset of cells were expressing beta lactamase. Such cells may also express other proteins, such as the proteins of the present invention at low, moderate, or high levels as well.

### BRIEF DESCRIPTION OF THE FIGURES

FIGS. 1A-B presents the nucleic acid sequence (SEQ ID NO:1) of the novel human GPCR, called BMSOTR herein. The standard one-letter abbreviation for amino acids is used to illustrate the deduced amino acid sequence. The polynucleotide sequence contains a sequence of 1840 nucleotides (SEQ ID NO:1), encoding a polypeptide of 383 amino acids (SEQ ID NO:2). The coding sequence (CDS) of BMSOTR comprises nucleotides 116 to 1264 of SEQ ID NO:1. An analysis of the BMSOTR polypeptide determined that it comprised the seven transmembrane domains (TM1 to TM7). Each transmembrane domain is represented by double underlining.

FIGS. 2A-B presents the nucleic acid sequence (SEQ ID NO:3) of a BMSOTR variant. The standard one-letter abbreviation for amino acids is used to illustrate the deduced amino acid sequence. The polynucleotide sequence contains a sequence of 1993 nucleotides (SEQ ID NO:3), encoding a polypeptide of 434 amino acids (SEQ ID NO:4). The coding sequence (CDS) of the BMSOTR variant comprises nucleotides 116 to 1417. An analysis of the BMSOTR variant polypeptide determined that it comprised the seven transmembrane domains (TM1 to TM7). Each transmembrane domain is represented by double underlining.

FIGS. 2A-BA presents an amino acid alignment between BMSOTR (SEQ ID NO:2) and the 7-transmembrane receptor Pfam model (SEQ ID NO:14); ‘Q’ indicates the query amino acid sequence (BMSOTR), and ‘T’ indicates the target amino acid sequence (7-transmembrane receptor Pfam model). FIGS. 2A-BB presents an amino acid alignment between BMSOTR variant (SEQ ID NO:4) and the 7-transmembrane receptor Pfam model (SEQ ID NO:14); ‘Q’ indicates the amino acid sequence of BMSOTR variant and ‘T’ indicates the amino acid sequence of the 7-transmembrane receptor Pfam model.

FIGS. 4A and 4B present the TMPRED prediction results of transmembrane domains for BMSOTR (FIGS. 2A-BA) and BMSOTR variant (FIGS. 2A-BB). Each amino acid is given a score, residues that score above 500 are strongly considered to be a transmembrane residue.

FIGS. 5A-C illustrates a sequence alignment of the amino acid sequence of the novel human GPCR BMSOTR (SEQ ID NO:2) and the novel human BMSOTR variant (SEQ ID NO:4) of the present invention with the closest species homologue OXYR_MOUSE OXYTOCIN RECEPTOR (SEQ ID NO:13; ACCESSION NO:3024340), a GPCR protein. The alignment was performed using the CLUSTALW algorithm using default parameters as described herein (Vector NTI suite of programs). The darkly shaded amino acids represent regions of matching identity. The lightly shaded amino acids represent regions of matching similarity. Dots (“•”) between residues indicate gapped regions of non-identity for the aligned polypeptides.

FIG. 6 shows the expression profile the novel human GPCR BMSOTR, as described in Example 6 herein.

FIG. 7 shows an expanded expression profile of the novel human transient GPCR, BMSOTR. The figure illustrates the relative expression level of BMSOTR amongst various mRNA tissue sources. As shown, the BMSOTR polypeptide was expressed predominately in the nervous system, specifically the cortex, hippocampus, with lower levels observed in other brain sub-regions. Expression data was obtained by measuring the steady state BMSOTR mRNA levels by quantitative PCR using the PCR primer pair provided as SEQ ID NO:53 and 54, and Taqman probe (SEQ ID NO:55) as described in Example 7 herein.

Table I provides a summary of various conservative substitutions encompassed by the present invention.

Table II provides a summary of the novel polypeptides and their encoding polynucleotides of the present invention.

### DETAILED DESCRIPTION OF THE INVENTION

The present invention provides a novel human GPCR (BMSOTR) gene (i.e., polynucleotide or nucleic acid sequence), (SEQ ID NO:1) which encodes a BMSOTR protein (polypeptide), (SEQ ID NO:2), preferably the full-length BMSOTR polypeptide. Based on percent sequence identity analysis, the protein BMSOTR has been determined to be a novel GPCR, in particular, a newly discovered oxytocin receptor. The BMSOTR is also referred to as “OXYTOCIN_CAND”, and “GPCR-170”.

The present invention also provides a BMSOTR variant gene (SEQ ID NO:3) which encodes a BMSOTR variant protein (SEQ ID NO:4). The BMSOTR variant protein or polypeptide contains additional amino acid residues 151-201 of the variant polypeptide sequence and amino acid changes at residues 206 and 207 corresponding to residues 155 and 156 of SEQ ID NO:2. All references to “BMSOTR” shall be construed to apply to BMSOTR, and the BMSOTR variant, unless otherwise specified herein.”

The invention further relates to fragments and portions of the novel BMSOTR nucleic acid sequence and its encoded amino acid sequence (peptides and polypeptides). Preferably, the fragments and portions of the BMSOTR polypeptide are functional or active. The invention also provides methods of using the novel BMSOTR polynucleotide sequence and the encoded BMSOTR polypeptide for diagnosis, genetic screening and treatment of diseases, disorders, conditions, or syndromes associated with BMSOTR and BMSOTR activity and function. The BMSOTR polynucleotide and polypeptide, may be involved in a variety of diseases, disorders and conditions associated with BMSOTR activity, which include, but are not limited to, immune-related disorders, acute heart failure, hypotension, hypertension, endocrinal diseases, growth disorders, neuropathic pain, obesity, anorexia, Prader-Willi syndrome, cancers, bulimia, asthma, Parkinson's disease, Alzheimer's disease, osteoporosis, angina pectoris, myocardial infarction, psychotic, metabolic, cardiovascular, reproductive and neurological disorders. Reproductive, endocrine, and neurological system-related conditions are particularly relevant.

### Definitions

The following definitions are provided to more fully describe the present invention in its various aspects. The definitions are intended to be useful for guidance and elucidation, and are not intended to limit the disclosed invention or its embodiments.

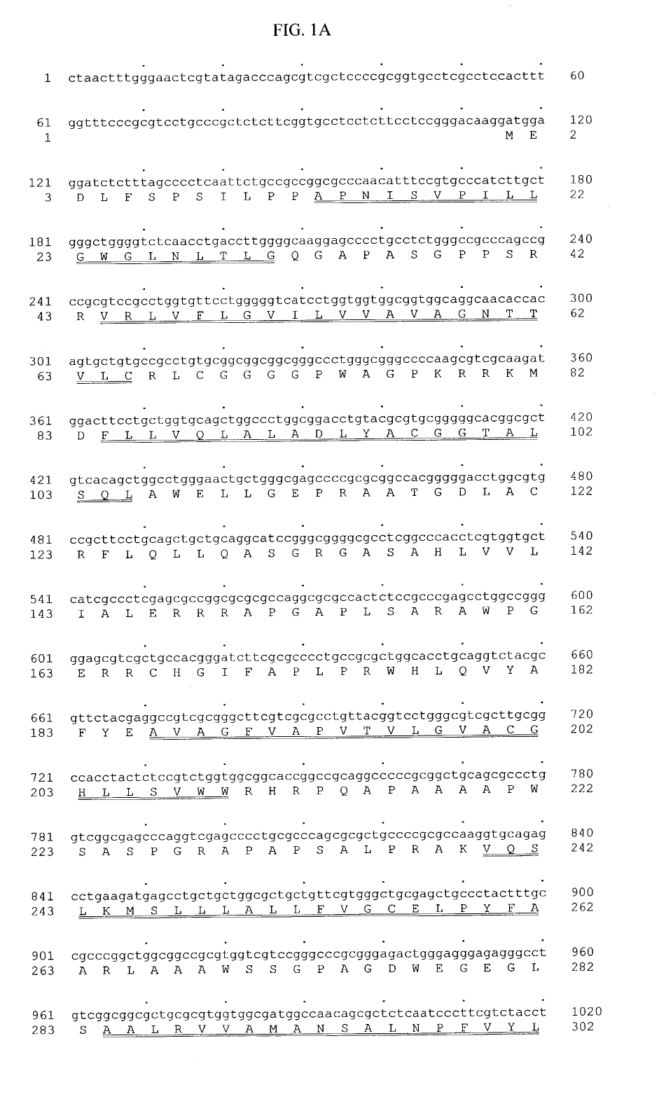

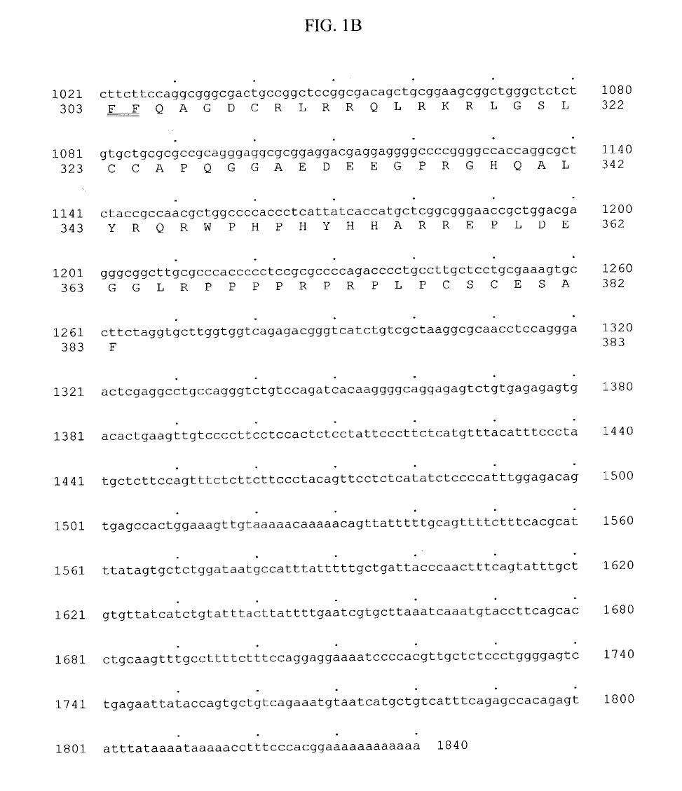

“Amino acid sequence” as used herein can refer to an oligopeptide, peptide, polypeptide, or protein sequence, and fragments or portions thereof, as well as to naturally occurring or synthetic molecules, preferably isolated polypeptides of BMSOTR. Amino acid sequence fragments are typically from about 4 to about 30, preferably from about 5 to about 15, more preferably from about 5 to about 15 amino acids in length and preferably retain the biological activity or function of a BMSOTR polypeptide. As will be appreciated by the skilled practitioner, should the amino acid fragment comprise an antigenic epitope, for example, biological function per se need not be maintained. The BMSOTR amino acid sequence of this invention is set forth in SEQ ID NO:2 and as illustrated in FIGS. 1A-B. The terms BMSOTR polypeptide and BMSOTR protein are used interchangeably herein to refer to the encoded product of the BMSOTR nucleic acid sequence according to the present invention.

Isolated BMSOTR polypeptide refers to the amino acid sequence of substantially purified BMSOTR, which may be obtained from any species, preferably mammalian, and more preferably, human, and from a variety of sources, including natural, synthetic, semi-synthetic, or recombinant. More particularly, the BMSOTR polypeptide of this invention is identified in SEQ ID NO:2. Fragments, preferably functional fragments, of the BMSOTR polypeptide are also embraced by the present invention.

“Similar” amino acids are those which have the same or similar physical properties and in many cases, the function is conserved with similar residues. For example, amino acids lysine and arginine are similar; while residues such as proline and cysteine do not share any physical property and are not considered to be similar.

The term “consensus” refers to a sequence that reflects the most common choice of base or amino acid at each position among a series of related DNA, RNA or protein sequences. Areas of particularly good agreement often represent conserved functional domains.

A “variant” of the BMSOTR polypeptide refers to an amino acid sequence that is altered by one or more amino acids. The variant may have “conservative” changes, in which a substituted amino acid has similar structural or chemical properties, e.g., replacement of leucine with isoleucine. More rarely, a variant may have “nonconservative” changes, for example, replacement of a glycine with a tryptophan. The encoded protein may also contain deletions, insertions, or substitutions of amino acid residues, which produce a silent change and result in a functionally equivalent BMSOTR protein. Deliberate amino acid substitutions may be made on the basis of similarity in polarity, charge, solubility, hydrophobicity, hydrophilicity, and/or the amphipathic nature of the residues, as long as the biological activity of BMSOTR protein is retained. For example, negatively charged amino acids may include aspartic acid and glutamic acid; positively charged amino acids may include lysine and arginine; and amino acids with uncharged polar head groups having similar hydrophilicity values may include leucine, isoleucine, and valine; glycine and alanine; asparagine and glutamine; serine and threonine; and phenylalanine and tyrosine. Guidance in determining which amino acid residues may be substituted, inserted, or deleted without abolishing functional biological or immunological activity may be found using computer programs well known in the art, for example, DNASTAR, Inc. software (Madison, Wis.).

The invention encompasses polypeptides having a lower degree of identity but having sufficient similarity so as to perform one or more of the same functions performed by the polypeptide of the present invention. Similarity is determined by conserved amino acid substitution. Such substitutions are those that substitute a given amino acid in a polypeptide by another amino acid of like characteristics (e.g., chemical properties). According to Cunningham et al above, such conservative substitutions are likely to be phenotypically silent. Additional guidance concerning which amino acid changes are likely to be phenotypically silent are found in Bowie et al., Science 247:1306-1310 (1990).

Tolerated conservative amino acid substitutions of the present invention involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

In addition, the present invention also encompasses the conservative substitutions provided in Table I below.

TABLE I

| For Amino | | |
|---|---|---|
| Acid | Code | Replace with any of: |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, β-Ala, Acp |
| Isoleucine | I | D-Ile, Val, D-Val, Leu, D-Leu, Met, D-Met |
| Leucine | L | D-Leu, Val, D-Val, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, D- |
| | | Trp, Trans-3,4, or 5-phenylproline, cis-3,4, or 5- |
| | | phenylproline |
| Proline | P | D-Pro, L-1-thioazolidine-4-carboxylic acid, D- or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, D-Met, Met(O), |
| | | D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Aside from the uses described above, such amino acid substitutions may also increase protein or peptide stability. The invention encompasses amino acid substitutions that contain, for example, one or more non-peptide bonds (which replace the peptide bonds) in the protein or peptide sequence. Also included are substitutions that include amino acid residues other than naturally occurring L-amino acids, e.g., D-amino acids or non-naturally occurring or synthetic amino acids, e.g., β or γ amino acids.

Both identity and similarity can be readily calculated by reference to the following publications: Computational Molecular Biology, Lesk, A. M., ed., Oxford University Press, New York, 1988; Biocomputing: Informatics and Genome Projects, Smith, D. W., ed., Academic Press, New York, 1993; Informatics Computer Analysis of Sequence Data, Part 1, Griffin, A. M., and Griffin, H. G., eds., Humana Press, New Jersey, 1994; Sequence Analysis in Molecular Biology, von Heinje, G., Academic 15 Press, 1987; and Sequence Analysis Primer, Gribskov, M. and Devereux, J., eds., M Stockton Press, New York, 1991.

In addition, the present invention also encompasses substitution of amino acids based upon the probability of an amino acid substitution resulting in conservation of function. Such probabilities are determined by aligning multiple genes with related function and assessing the relative penalty of each substitution to proper gene function. Such probabilities are often described in a matrix and are used by some algorithms (e.g., BLAST, CLUSTALW, GAP, etc.) in calculating percent similarity wherein similarity refers to the degree by which one amino acid may substitute for another amino acid without lose of function. An example of such a matrix is the PAM250 or BLOSUM62 matrix.

Aside from the canonical chemically conservative substitutions referenced above, the invention also encompasses substitutions which are typically not classified as conservative, but that may be chemically conservative under certain circumstances. Analysis of enzymatic catalysis for proteases, for example, has shown that certain amino acids within the active site of some enzymes may have highly perturbed pKa's due to the unique microenvironment of the active site. Such perturbed pKa's could enable some amino acids to substitute for other amino acids while conserving enzymatic structure and function. Examples of amino acids that are known to have amino acids with perturbed pKa's are the Glu-35 residue of Lysozyme, the Ile-16 residue of Chymotrypsin, the His-159 residue of Papain, etc. The conservation of function relates to either anomalous protonation or anomalous deprotonation of such amino acids, relative to their canonical, non-perturbed pKa. The pKa perturbation may enable these amino acids to actively participate in general acid-base catalysis due to the unique ionization environment within the enzyme active site. Thus, substituting an amino acid capable of serving as either a general acid or general base within the microenvironment of an enzyme active site or cavity, as may be the case, in the same or similar capacity as the wild-type amino acid, would effectively serve as a conservative amino substitution.

The term “mimetic”, as used herein, refers to a molecule, having a structure which is developed from knowledge of the structure of the BMSOTR protein, or portions thereof, and as such, is able to affect some or all of the actions of the BMSOTR protein. A mimetic may comprise a synthetic peptide or an organic molecule.

“Nucleic acid or polynucleotide sequence”, as used herein, refers to an isolated oligonucleotide (“oligo”), nucleotide, or polynucleotide, and fragments thereof, and to DNA or RNA of genomic or synthetic origin which may be single- or double-stranded, and represent the sense or anti-sense strand, preferably of BMSOTR. By way of non-limiting example, fragments include nucleic acid sequences that are 20-60 nucleotides in length, or greater, and preferably include fragments that are at least 50-100 nucleotides, or which are at least 1000 nucleotides or greater in length. The BMSOTR nucleic acid sequence of this invention is specifically identified in SEQ ID NO:1, and is illustrated in FIGS. 1A-B.

An “allele” or “allelic sequence” is an alternative form of the BMSOTR nucleic acid sequence. Alleles may result from at least one mutation in the BMSOTR nucleic acid sequence and may yield altered mRNAs or polypeptides whose structure or function may or may not be altered. Any given gene, whether natural or recombinant, may have none, one, or many allelic forms. Common mutational changes, which give rise to alleles, are generally ascribed to natural deletions, additions, or substitutions of nucleotides. Each of these types of changes may occur alone, or in combination with the others, one or more times in a given sequence.

“Peptide nucleic acid” (PNA) refers to an antisense molecule or anti-gene agent which comprises an oligonucleotide (“oligo”) linked via an amide bond, similar to the peptide backbone of amino acid residues. PNAs typically comprise oligos of at least 5 nucleotides linked via amide bonds. PNAs may or may not terminate in positively charged amino acid residues to enhance binding affinities to DNA. Such amino acids include, for example, lysine and arginine, among others. These small molecules stop transcript elongation by binding to their complementary strand of nucleic acid (P. E. Nielsen et al., 1993, Anticancer Drug Des., 8:53-63). PNA may be pegylated to extend their lifespan in the cell where they preferentially bind to complementary single stranded DNA and RNA.

“Oligonucleotides” or “oligomers”, as defined herein, refer to a BMSOTR nucleic acid sequence comprising contiguous nucleotides of at least about 5 nucleotides to about 60 nucleotides, preferably at least about 8 to 10 nucleotides in length, more preferably at least about 12 nucleotides in length, for example, about 15 to 35 nucleotides, or about 15 to 25 nucleotides, or about 20 to 35 nucleotides, which can be typically used as probes or primers, for example, in PCR amplification assays, hybridization assays, or in microarrays. It will be understood that the term oligonucleotide is substantially equivalent to the terms primer, probe, or amplimer, as commonly defined in the art. Examples of BMSOTR primers of this invention are set forth SEQ ID NOS:5-7.

The term “antisense” refers to nucleotide sequences, and compositions containing nucleic acid sequences, which are complementary to a specific DNA or RNA sequence. The term “antisense strand” is used in reference to a nucleic acid strand that is complementary to the “sense” strand. Antisense (i.e., complementary) nucleic acid molecules include PNAs and may be produced by any method, including synthesis or transcription. Once introduced into a cell, the complementary nucleotides combine with natural sequences produced by the cell to form duplexes, which block either transcription or translation. The designation “negative” is sometimes used in reference to the antisense strand, and “positive” is sometimes used in reference to the sense strand.

“Altered” nucleic acid sequences encoding a BMSOTR polypeptide include nucleic acid sequences containing deletions, insertions and/or substitutions of different nucleotides resulting in a polynucleotide that encodes the same or a functionally equivalent BMSOTR polypeptide. Altered nucleic acid sequences may further include polymorphisms of the polynucleotide encoding the BMSOTR polypeptide; such polymorphisms may or may not be readily detectable using a particular oligonucleotide probe.

The terms “Expressed Sequence Tag” or “EST” refers to the partial sequence of a cDNA insert which has been made by reverse transcription of mRNA extracted from a tissue, followed by insertion into a vector as known in the art (Adams, M. D., et al. Science (1991) 252:1651-1656; Adams, M. D. et al., Nature, (1992) 355:632634; Adams, M. D., et al., Nature (1995) 377 Supp:3-174).

The term “biologically active”, i.e., functional, refers to a protein or polypeptide or fragment thereof, having structural, regulatory, or biochemical functions of a naturally occurring molecule. Likewise, “immunologically active” refers to the capability of a natural, recombinant, or synthetic BMSOTR polypeptide, or an oligopeptide thereof, to induce a specific immune response in appropriate animals or cells, for example, to generate antibodies, to bind with specific antibodies, and/or to elicit a cellular immune response.

In a preferred embodiment, the functional activity displayed by a polypeptide encoded by a polynucleotide fragment of the invention may be one or more biological activities typically associated with the full-length polypeptide of the invention. Illustrative of these biological activities includes the fragments ability to bind to at least one of the same antibodies which bind to the full-length protein, the fragments ability to interact with at lease one of the same proteins which bind to the full-length, the fragments ability to elicit at least one of the same immune responses as the full-length protein (i.e., to cause the immune system to create antibodies specific to the same epitope, etc.), the fragments ability to bind to at least one of the same polynucleotides as the full-length protein, the fragments ability to bind to a receptor of the full-length protein, the fragments ability to bind to a ligand of the full-length protein, and the fragments ability to multimerize with the full-length protein. However, the skilled artisan would appreciate that some fragments may have biological activities which are desirable and directly inapposite to the biological activity of the full-length protein. The functional activity of polypeptides of the invention, including fragments, variants, derivatives, and analogs thereof can be determined by numerous methods available to the skilled artisan, some of which are described elsewhere herein.

It is another aspect of the present invention to provide modulators of the BMSOTR and/or BMSORT variant protein and BMSOTR and/or BMSORT variant peptide targets which can affect the function or activity of BMSOTR and/or BMSORT variant in a cell in which BMSOTR and/or BMSORT variant function or activity is to be modulated or affected. In addition, modulators of BMSOTR and/or BMSORT variant can affect downstream systems and molecules that are regulated by, or which interact with, BMSOTR and/or BMSORT variant in the cell. Modulators of BMSOTR and/or BMSORT variant include compounds, materials, agents, drugs, and the like, that antagonize, inhibit, reduce, block, suppress, diminish, decrease, or eliminate BMSOTR and/or BMSORT variant function and/or activity. Such compounds, materials, agents, drugs and the like can be collectively termed “antagonists”. Alternatively, modulators of BMSOTR and/or BMSORT variant include compounds, materials, agents, drugs, and the like, that agonize, enhance, increase, augment, or amplify BMSOTR and/or BMSORT variant function in a cell. Such compounds, materials, agents, drugs and the like can be collectively termed “agonists”.

An “agonist” refers to a molecule which, when bound to, or associated with, a BMSOTR polypeptide, or a functional fragment thereof, increases or prolongs the duration of the effect of the BMSOTR polypeptide. Agonists may include proteins, nucleic acids, carbohydrates, or any other molecules that bind to and modulate the effect of the BMSOTR polypeptide. Agonists typically enhance, increase, or augment the function or activity of a BMSOTR molecule.

An “antagonist” refers to a molecule which, when bound to, or associated with, a BMSOTR polypeptide, or a functional fragment thereof, decreases or inhibits the amount or duration of the biological or immunological activity of BMSOTR polypeptide. Antagonists may include proteins, nucleic acids, carbohydrates, antibodies, or any other molecules that decrease or reduce the effect of a BMSOTR polypeptide. Antagonists typically, diminish, inhibit, or reduce the function or activity of a BMSOTR molecule.

As used herein the terms “modulate” or “modulates” refer to an increase or decrease in the amount, quality or effect of a particular activity, DNA, RNA, or protein. The definition of “modulate” or “modulates” as used herein is meant to encompass agonists and/or antagonists of a particular activity, DNA, RNA, or protein.

The terms “complementary” or “complementarity” refer to the natural binding of polynucleotides under permissive salt and temperature conditions by base pairing. For example, the sequence “A-G-T” binds to the complementary sequence “T-C-A”. Complementarity between two single-stranded molecules may be “partial”, in which only some of the nucleic acids bind, or it may be “complete” when total complementarity exists between single stranded molecules. The degree of complementarity between nucleic acid strands has significant effects on the efficiency and strength of hybridization between nucleic acid strands. This is of particular importance in amplification reactions, which depend upon binding between nucleic acids strands, as well as in the design and use of PNA molecules.

The term “homology” refers to a degree of complementarity. There may be partial homology or complete homology, wherein complete homology is equivalent to identity. A partially complementary sequence that at least partially inhibits an identical sequence from hybridizing to a target nucleic acid is referred to as the functional term “substantially homologous”. The inhibition of hybridization of the completely complementary sequence to the target sequence may be examined using a hybridization assay (for example, Southern or Northern blot, solution hybridization, and the like) under conditions of low stringency. A substantially homologous sequence or probe will compete for and inhibit the binding (i.e., the hybridization) of a completely homologous sequence or probe to the target sequence under conditions of low stringency. Nonetheless, conditions of low stringency do not permit nonspecific binding; low stringency conditions require that the binding of two sequences to one another be a specific (i.e., selective) interaction. The absence of non-specific binding may be tested by the use of a second target sequence which lacks even a partial degree of complementarity (for example, less than about 30% identity). In the absence of non-specific binding, the probe will not hybridize to the second non-complementary target sequence.

The present invention is also directed to polynucleotide sequences which comprise, or alternatively consist of, a polynucleotide sequence which is at least about 80%, 85%, 88.0%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

Another aspect of the invention provides an isolated nucleic acid molecule comprising, or alternatively, consisting of, a polynucleotide having a nucleotide sequence selected from the group consisting of: (a) a nucleotide sequence encoding a BMSOTR and/or BMSOTR variant related polypeptide having an amino acid sequence as shown in the sequence listing and descried in Table II; (b) a nucleotide sequence encoding a mature BMSOTR and/or BMSOTR variant related polypeptide having the amino acid sequence as shown in the sequence listing and descried in Table II; (c) a nucleotide sequence encoding a biologically active fragment of a BMSOTR and/or BMSOTR variant related polypeptide having an amino acid sequence as shown in the sequence listing and descried in Table II; (d) a nucleotide sequence encoding an antigenic fragment of a BMSOTR and/or BMSOTR variant related polypeptide having an amino acid sequence as shown in the sequence listing and descried in Table II; (e) a nucleotide sequence encoding a BMSOTR and/or BMSOTR variant related polypeptide comprising the complete amino acid sequence encoded by a human cDNA in a cDNA plasmid contained in the ATCC Deposit and described in Table II; (f) a nucleotide sequence encoding a mature BMSOTR and/or BMSOTR variant related polypeptide having an amino acid sequence encoded by a human cDNA in a cDNA plasmid contained in the ATCC Deposit and described in Table II: (g) a nucleotide sequence encoding a biologically active fragment of a BMSOTR and/or BMSOTR variant related polypeptide having an amino acid sequence encoded by a human cDNA in a cDNA plasmid contained in the ATCC Deposit and described in Table II; (h) a nucleotide sequence encoding an antigenic fragment of a BMSOTR and/or BMSOTR variant related polypeptide having an amino acid sequence encoded by a human cDNA in a cDNA plasmid contained in the ATCC deposit and described in Table II; (i) a nucleotide sequence complimentary to any of the nucleotide sequences in (a), (b), (c), (d), (e), (f), (g), or (h) above.

The present invention is also directed to nucleic acid molecules which comprise, or alternatively, consist of, a nucleotide sequence which is at least about 80%, 85%, 88.0%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, any of the nucleotide sequences in (a), (b), (c), (d), (e), (f), (g), or (h), above.

The present invention encompasses polypeptide sequences which comprise, or alternatively consist of, an amino acid sequence which is at least about 80%, 85%, 87.4%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, the following non-limited examples, the polypeptide sequence identified as SEQ ID NO:2 and/or 4, the polypeptide sequence encoded by a cDNA provided in the deposited clone, and/or polypeptide fragments of any of the polypeptides provided herein. Polynucleotides encoded by these nucleic acid molecules are also encompassed by the invention. In another embodiment, the invention encompasses nucleic acid molecules which comprise, or alternatively, consist of a polynucleotide which hybridizes under stringent conditions, or alternatively, under lower stringency conditions, to a polynucleotide in (a), (b), (c), (d), (e), (f), (g), or (h), above. Polynucleotides which hybridize to the complement of these nucleic acid molecules under stringent hybridization conditions or alternatively, under lower stringency conditions, are also encompassed by the invention, as are polypeptides encoded by these polypeptides.

The present invention is also directed to polypeptides which comprise, or alternatively consist of, an amino acid sequence which is at least about 80%, 85%, 87.4%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for example, the polypeptide sequence shown in SEQ ID NO:2 and/or 4, a polypeptide sequence encoded by the nucleotide sequence in SEQ ID NO:1 and/or 3, a polypeptide sequence encoded by the cDNA in cDNA plasmid:Z, and/or polypeptide fragments of any of these polypeptides (e.g., those fragments described herein). Polynucleotides which hybridize to the complement of the nucleic acid molecules encoding these polypeptides under stringent hybridization conditions or alternatively, under lower stringency conditions, are also encompasses by the present invention, as are the polypeptides encoded by these polynucleotides.

By a nucleic acid having a nucleotide sequence at least, for example, 95% “identical” to a reference nucleotide sequence of the present invention, it is intended that the nucleotide sequence of the nucleic acid is identical to the reference sequence except that the nucleotide sequence may include up to five point mutations per each 100 nucleotides of the reference nucleotide sequence encoding the polypeptide. In other words, to obtain a nucleic acid having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. The query sequence may be an entire sequence referenced in Table II, the ORF (open reading frame), or any fragment specified as described herein.

As a practical matter, whether any particular nucleic acid molecule or polypeptide is at least about 80%, 85%, 87.4%, 88.0%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both DNA sequences. An RNA sequence can be compared by converting U's to T's. However, the CLUSTALW algorithm automatically converts U's to T's when comparing RNA sequences to DNA sequences. The result of said global sequence alignment is in percent identity. Preferred parameters used in a CLUSTALW alignment of DNA sequences to calculate percent identity via pairwise alignments are: Matrix=IUB, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of 5′ or 3′ deletions, not because of internal deletions. If only the local pairwise percent identity is required, no manual correction is needed. However, a manual correction may be applied to determine the global percent identity from a global polynucleotide alignment. Percent identity calculations based upon global polynucleotide alignments are often preferred since they reflect the percent identity between the polynucleotide molecules as a whole (i.e., including any polynucleotide overhangs, not just overlapping regions), as opposed to, only local matching polynucleotides. Manual corrections for global percent identity determinations are required since the CLUSTALW program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the CLUSTALW sequence alignment. This percentage is then subtracted from the percent identity, calculated by the above CLUSTALW program using the specified parameters, to arrive at a final percent identity score. This corrected score may be used for the purposes of the present invention. Only bases outside the 5′ and 3′ bases of the subject sequence, as displayed by the CLUSTALW alignment, which are not matched/aligned with the query sequence, are calculated for the purposes of manually adjusting the percent identity score.

For example, a 90 base subject sequence is aligned to a 100 base query sequence to determine percent identity. The deletions occur at the 5′ end of the subject sequence and therefore, the CLUSTALW alignment does not show a matched/alignment of the first 10 bases at 5′ end. The 10 unpaired bases represent 10% of the sequence (number of bases at the 5′ and 3′ ends not matched/total number of bases in the query sequence) so 10% is subtracted from the percent identity score calculated by the CLUSTALW program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by CLUSTALW is not manually corrected. Once again, only bases 5′ and 3′ of the subject sequence which are not matched/aligned with the query sequence are manually corrected for. No other manual corrections are required for the purposes of the present invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least about 80%, 85%, 87.4%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% identical to, for instance, an amino acid sequence referenced in Table II (SEQ ID NO:2) or to the amino acid sequence encoded by cDNA contained in a deposited clone, can be determined conventionally using known computer programs. A preferred method for determining the best overall match between a query sequence (a sequence of the present invention) and a subject sequence, also referred to as a global sequence alignment, can be determined using the CLUSTALW computer program (Thompson, J. D., et al., Nucleic Acids Research, 2(22):4673-4680, (1994)), which is based on the algorithm of Higgins, D. G., et al., Computer Applications in the Biosciences (CABIOS), 8(2):189-191, (1992). In a sequence alignment the query and subject sequences are both amino acid sequences. The result of said global sequence alignment is in percent identity. Preferred parameters used in a CLUSTALW alignment of polypeptide sequences to calculate percent identity via pairwise alignments are: Matrix=BLOSUM, k-tuple=1, Number of Top Diagonals=5, Gap Penalty=3, Gap Open Penalty 10, Gap Extension Penalty=0.1, Scoring Method=Percent, Window Size=5 or the length of the subject nucleotide sequence, whichever is shorter. For multiple alignments, the following CLUSTALW parameters are preferred: Gap Opening Penalty=10; Gap Extension Parameter=0.05; Gap Separation Penalty Range=8; End Gap Separation Penalty=Off; % Identity for Alignment Delay=40%; Residue Specific Gaps:Off; Hydrophilic Residue Gap=Off; and Transition Weighting=0. The pairwise and multple alignment parameters provided for CLUSTALW above represent the default parameters as provided with the AlignX software program (Vector NTI suite of programs, version 6.0).

The present invention encompasses the application of a manual correction to the percent identity results, in the instance where the subject sequence is shorter than the query sequence because of N- or C-terminal deletions, not because of internal deletions. If only the local pairwise percent identity is required, no manual correction is needed. However, a manual correction may be applied to determine the global percent identity from a global polypeptide alignment. Percent identity calculations based upon global polypeptide alignments are often preferred since they reflect the percent identity between the polypeptide molecules as a whole (i.e., including any polypeptide overhangs, not just overlapping regions), as opposed to, only local matching polypeptides. Manual corrections for global percent identity determinations are required since the CLUSTALW program does not account for N- and C-terminal truncations of the subject sequence when calculating percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the CLUSTALW sequence alignment. This percentage is then subtracted from the percent identity, calculated by the above CLUSTALW program using the specified parameters, to arrive at a final percent identity score. This final percent identity score is what may be used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence.

For example, a 90 amino acid residue subject sequence is aligned with a 100 residue query sequence to determine percent identity. The deletion occurs at the N-terminus of the subject sequence and therefore, the CLUSTALW alignment does not show a matching/alignment of the first 10 residues at the N-terminus. The 10 unpaired residues represent 10% of the sequence (number of residues at the N- and C-termini not matched/total number of residues in the query sequence) so 10% is subtracted from the percent identity score calculated by the CLUSTALW program. If the remaining 90 residues were perfectly matched the final percent identity would be 90%. In another example, a 90 residue subject sequence is compared with a 100 residue query sequence. This time the deletions are internal deletions so there are no residues at the N- or C-termini of the subject sequence, which are not matched/aligned with the query. In this case the percent identity calculated by CLUSTALW is not manually corrected. Once again, only residue positions outside the N- and C-terminal ends of the subject sequence, as displayed in the CLUSTALW alignment, which are not matched/aligned with the query sequence are manually corrected for. No other manual corrections are required for the purposes of the present invention.

In addition to the above method of aligning two or more polynucleotide or polypeptide sequences to arrive at a percent identity value for the aligned sequences, it may be desirable in some circumstances to use a modified version of the CLUSTALW algorithm which takes into account known structural features of the sequences to be aligned, such as for example, the SWISS-PROT designations for each sequence. The result of such a modifed CLUSTALW algorithm may provide a more accurate value of the percent identity for two polynucleotide or polypeptide sequences. Support for such a modified version of CLUSTALW is provided within the CLUSTALW algorithm and would be readily appreciated to one of skill in the art of bioinformatics.

Those having skill in the art will know how to determine percent identity between/among sequences using, for example, algorithms such as those used in the GAP computer program (S. B. Needleman and C. D. Wunsch. A general method applicable to the search for similarities in the amino acid sequence of two proteins. J. Mol. Biol. 48(3):443-53, 1970) or based on the CLUSTALW computer program (J. D. Thompson et al., 1994, Nuc. Acids Res., 2(22):4673-4680), or FASTDB, (Brutlag et al., 1990, Comp. App. Biosci., 6:237-245), as known in the art. Although the FASTDB algorithm typically does not consider internal non-matching deletions or additions in sequences, i.e., gaps, in its calculation, this can be corrected manually to avoid an overestimation of the % identity. GAP and CLUSTALW, however, do take sequence gaps into account in their identity calculations.

Also available to those having skill in this art are the BLAST and BLAST 2.0 algorithms (Altschul et al., 1977, Nuc. Acids Res., 25:3389-3402 and Altschul et al., 1990, J. Mol. Biol., 215:403-410). The BLASTN program for nucleic acid sequences uses as defaults a wordlength (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff & Henikoff, 1989, Proc. Natl. Acad. Sci., USA, 89:10915) uses alignments (B) of 50, expectation (E) of 10, M=5, N=4, and a comparison of both strands.

The term “hybridization” refers to any process by which a strand of nucleic acids binds with a complementary strand through base pairing. The term “hybridization complex” refers to a complex formed between two nucleic acid sequences by virtue of the formation of hydrogen bonds between complementary G and C bases and between complementary A and T bases. The hydrogen bonds may be further stabilized by base stacking interactions. The two complementary nucleic acid sequences hydrogen bond in an anti-parallel configuration. A hybridization complex may be formed in solution (for example, Cₒt or Rₒt analysis), or between one nucleic acid sequence present in solution and another nucleic acid sequence immobilized on a solid phase or support (for example, membranes, filters, chips, pins, or glass slides, or any other appropriate substrate to which cells or their nucleic acids have been affixed).

The terms “stringency” or “stringent conditions” refer to the conditions for hybridization as defined by nucleic acid composition, salt, and temperature. These conditions are well known in the art and may be altered to identify and/or detect identical or related polynucleotide sequences in a sample. A variety of equivalent conditions comprising either low, moderate, or high stringency depend on factors such as the length and nature of the sequence (DNA, RNA, base composition), reaction milieu (in solution or immobilized on a solid substrate), nature of the target nucleic acid (DNA, RNA, base composition), concentration of salts and the presence or absence of other reaction components (for example, formamide, dextran sulfate and/or polyethylene glycol) and reaction temperature (within a range of from about 5° C. below the melting temperature of the probe to about 20° C. to 25° C. below the melting temperature). One or more factors may be varied to generate conditions, either low or high stringency that is different from but equivalent to the aforementioned conditions.

As will be understood by those of skill in the art, the stringency of hybridization may be altered in order to identify or detect identical or related polynucleotide sequences. As will be further appreciated by the skilled practitioner, the melting temperature, Tₘ, can be approximated by the formulas as well known in the art, depending on a number of parameters, such as the length of the hybrid or probe in number of nucleotides, or hybridization buffer ingredients and conditions (see, for example, T. Maniatis et al., Molecular Cloning: A Laboratory Manual, Cold Spring Harbor Laboratory, Cold Spring Harbor, N.Y., 1982 and J. Sambrook et al., Molecular Cloning: A Laboratory Manual, Cold Spring Harbor Laboratory, Cold Spring Harbor, N.Y., 1989; Current Protocols in Molecular Biology, Eds. F. M. Ausubel et al., Vol. 1, “Preparation and Analysis of DNA”, John Wiley and Sons, Inc., 1994-1995, Suppls. 26, 29, 35 and 42; pp. 2.10.7-2.10.16; G. M. Wahl and S. L. Berger (1987; Methods Enzymol. 152:399-407); and A. R. Kimmel, 1987; Methods of Enzymol. 152:507-511).

As a general guide, Tₘ decreases approximately 1° C.-1.5° C. with every 1% decrease in sequence homology. Also, in general, the stability of a hybrid is a function of sodium ion concentration and temperature. Typically, the hybridization reaction is initially performed under conditions of low stringency, followed by washes of varying, but higher stringency. Reference to hybridization stringency, for example, high, moderate, or low stringency, typically relates to such washing conditions. It is to be understood that the low, moderate and high stringency hybridization or washing conditions can be varied using a variety of ingredients, buffers and temperatures well known to and practiced by the skilled artisan.

A “composition”, as defined herein, refers broadly to any composition containing a BMSOTR polynucleotide, polypeptide, derivative, or mimetic thereof, or antibodies thereto. The composition may comprise a dry formulation or an aqueous solution. Compositions comprising the BMSOTR polynucleotide sequence (SEQ ID NO:1) encoding BMSOTR polypeptide (SEQ ID NO:2), or fragments thereof, may be employed as hybridization probes. The probes may be stored in a freeze-dried form and may be in association with a stabilizing agent such as a carbohydrate. In hybridizations, the probe may be employed in an aqueous solution containing salts (for example, NaCl), detergents or surfactants (for example, SDS) and other components (for example, Denhardt's solution, dry milk, salmon sperm DNA, and the like).

The term “substantially purified” refers to nucleic acid sequences or amino acid sequences that are removed from their natural environment, isolated or separated, and are at least 60% free, preferably 75% to 85% free, and most preferably 90% to 95%, or greater, free from other components with which they are naturally associated.

The term “sample”, or “biological sample”, is meant to be interpreted in its broadest sense. A non-limiting example of a biological sample suspected of containing a BMSOTR nucleic acid encoding BMSOTR protein, or fragments thereof, or a BMSOTR protein itself, may comprise, but is not limited to, a body fluid, an extract from cells or tissue, chromosomes isolated from a cell (for example, a spread of metaphase chromosomes), organelle, or membrane isolated from a cell, a cell, nucleic acid such as genomic BMSOTR DNA (in solution or bound to a solid support such as, for example, for Southern analysis), BMSOTR RNA (in solution or bound to a solid support such as for Northern analysis), BMSOTR cDNA (in solution or bound to a solid support), a tissue, a tissue print, and the like.

“Transformation” or transfection refers to a process by which exogenous DNA, preferably BMSOTR DNA, enters and changes a recipient cell. It may occur under natural or artificial conditions using various methods well known in the art. Transformation may rely on any known method for the insertion of foreign nucleic acid sequences into a prokaryotic or eukaryotic host cell. The method is selected based on the type of host cell being transformed and may include, but is not limited to, viral infection, electroporation, heat shock, lipofection, and partial bombardment. Such “transformed” cells include stably transformed cells in which the inserted DNA is capable of replication either as an autonomously replicating plasmid or as part of the host chromosome. Transformed cells also include those cells, which transiently express the inserted DNA or RNA for limited periods of time.

The term “correlates with expression of a polynucleotide” indicates that the detection of the presence of ribonucleic acid that is similar to the nucleic acid sequence of BMSOTR by Northern analysis is indicative of the presence of mRNA encoding BMSOTR polypeptide (SEQ ID NO:2) in a sample and thereby correlates with expression of the transcript from the polynucleotide encoding the protein.

An alteration in the polynucleotide of SEQ ID NO:1 comprises any alteration in the sequence of the polynucleotide encoding BMSOTR polypeptide, including deletions, insertions, and point mutations that may be detected using hybridization assays. Included within this definition is the detection of alterations to the genomic DNA sequence which encodes the BMSOTR polypeptide (e.g., by alterations in the pattern of restriction fragment length polymorphisms capable of hybridizing to nucleic acid sequences of SEQ ID NO:1), the inability of a selected fragment of SEQ ID NO:1 to hybridize to a sample of genomic DNA (e.g., using allele-specific oligonucleotide probes), and improper or unexpected hybridization, such as hybridization to a locus other than the normal chromosomal locus for the polynucleotide sequence encoding the BMSOTR polypeptide (e.g., using fluorescent in situ hybridization (FISH) to metaphase chromosome spreads).

The term “antibody” refers to intact molecules as well as fragments thereof, such as Fab, F(ab′)₂, Fv, which are capable of binding an epitopic or antigenic determinant. Antibodies that bind to a BMSOTR polypeptide can be prepared using intact polypeptides or fragments containing small peptides of interest or prepared recombinantly for use as the immunizing antigen. The polypeptide or oligopeptide used to immunize an animal can be derived from the transition of RNA or synthesized chemically, and can be conjugated to a carrier protein, if desired. Commonly used carriers that are chemically coupled to peptides include, but are not limited to, bovine serum albumin (BSA), keyhole limpet hemocyanin (KLH), and thyroglobulin. The coupled peptide is then used to immunize the animal (for example, a mouse, a rat, or a rabbit).

The term “humanized” antibody refers to antibody molecules in which amino acids have been replaced in the non-antigen binding regions (i.e., framework regions) of the immunoglobulin in order to more closely resemble a human antibody, while still retaining the original binding capability, for example, as described in U.S. Pat. No. 5,585,089 to C. L. Queen et al. In the present instance, humanized antibodies are preferably anti-BMSOTR specific antibodies.

The term “antigenic determinant” refers to that portion of a molecule that makes contact with a particular antibody (i.e., an epitope). When a protein or fragment of a protein, preferably a BMSOTR protein, is used to immunize a host animal, numerous regions of the protein may induce the production of antibodies which bind specifically to a given region or three-dimensional structure on the protein; these regions or structures are referred to an antigenic determinants. An antigenic determinant may compete with the intact antigen (i.e., the immunogen used to elicit the immune response) for binding to an antibody.

The terms “specific binding” or “specifically binding” refer to the interaction between a protein or peptide, preferably a BMSOTR protein, and a binding molecule, such as an agonist, an antagonist, or an antibody. The interaction is dependent upon the presence of a particular structure (i.e., an antigenic determinant or epitope) of the protein that is recognized by the binding molecule.

### DESCRIPTION OF THE INVENTION

The present invention provides a novel BMSOTR polynucleotide (SEQ ID NO:1) and its encoded BMSOTR polypeptide (SEQ ID NO:2). The BMSOTR according to this invention is preferably a full-length molecule. More specifically, the BMSOTR according to the invention is a GPCR family member. BMSOTR particularly belongs to the group of oxytocin and vasopressin receptor GPCRs.

GPCRs also include sensory receptors, chemokine receptors, orphan receptors, dopamine receptors, rhodopsin receptors, kinin receptors, N-formyl peptide receptors, opioid receptors, calcitonin receptors, adrenergic receptors, endothelin receptors, cAMP receptors, adenosine receptors, muscarinic receptors, acetylcholine receptors, serotonin receptors, histamine receptors, thrombin receptors, follicle stimulating hormone receptors, opsin receptors, endothelial differentiation gene-1 receptors, taste receptors, odorant receptors, or cytomegalovirus receptors.

The BMSOTR polynucleotide and/or polypeptide of this invention are useful for diagnosing diseases related to over- or under-expression of the BMSOTR protein. For example, such BMSOTR-associated diseases can be assessed by identifying mutations in the BMSOTR gene using BMSOTR probes or primers, or by determining BMSOTR protein or mRNA expression levels. A BMSOTR polypeptide is also useful for screening compounds which affect activity of the protein. The invention further encompasses the polynucleotide encoding the BMSOTR polypeptide and the use of the BMSOTR polynucleotide or polypeptide, or compositions thereof, in the screening, diagnosis, treatment, or prevention of disorders associated with aberrant or uncontrolled cellular growth and/or function, such as neoplastic diseases (for example, cancers and tumors). BMSOTR probes or primers can be used, for example, to screen for diseases associated with BMSOTR expression.

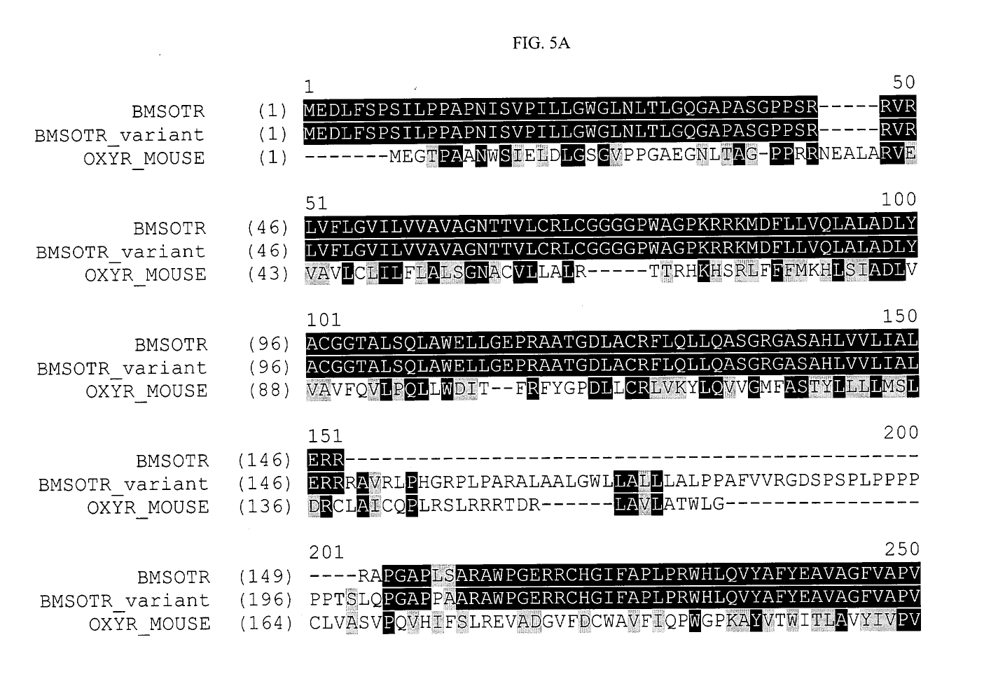

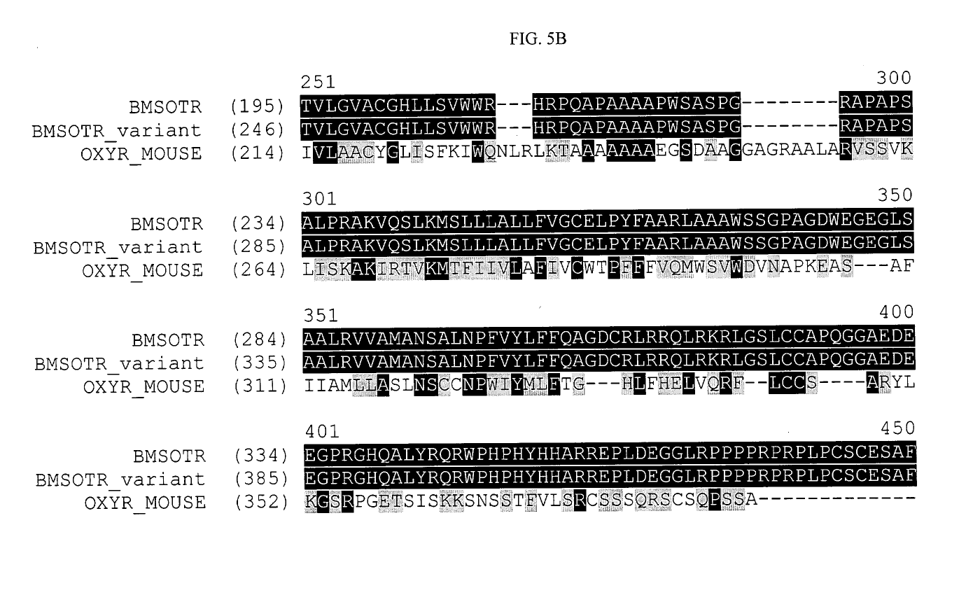

One embodiment of the present invention encompasses a novel BMSOTR polypeptide comprising the amino acid sequence of SEQ ID NO:2 as shown in FIGS. 1A-B. More specifically, the BMSOTR polypeptide of SEQ ID NO:2 is 383 amino acids in length and has 27% local amino acid sequence identity and 43% local amino acid sequence similarity, FIGS. 5A-B, with the GPCR, OXYR_MOUSE OXYTOCIN RECEPTOR (SEQ ID NO:13).

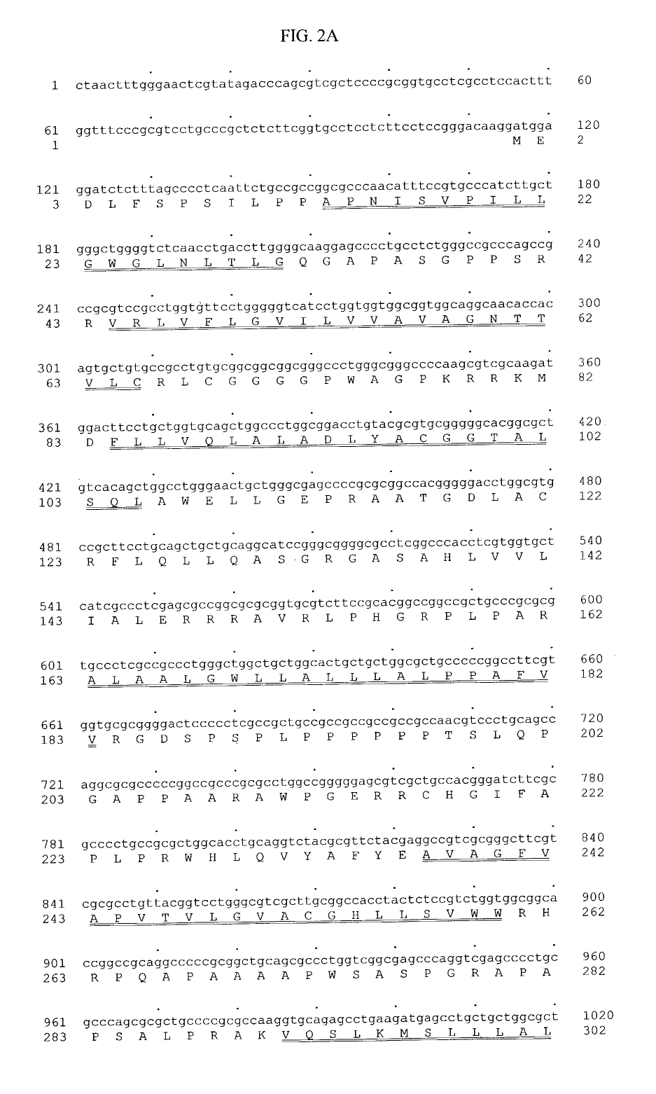

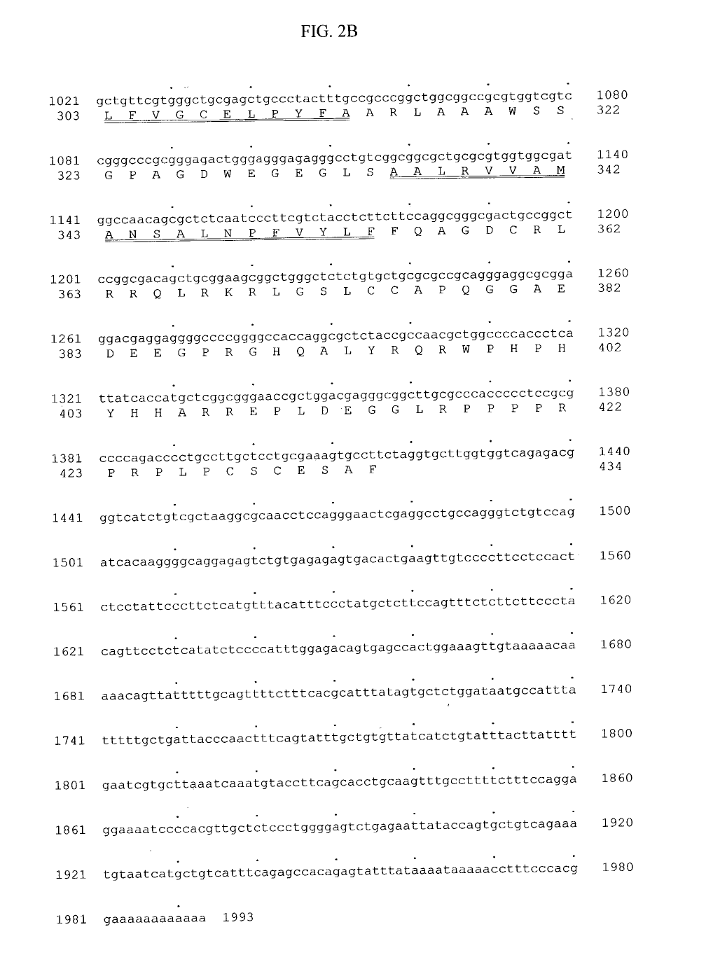

One embodiment of the present invention encompasses a novel BMSOTR variant polypeptide comprising the amino acid sequence of SEQ ID NO:4 as shown in FIGS. 2A-B. More specifically, the BMSOTR variant polypeptide of SEQ ID NO:4 is 434 amino acids in length and has 28% local amino acid sequence identity and 44% local amino acid sequence similarity, FIGS. 5A-B, with the GPCR, OXYR_MOUSE OXYTOCIN RECEPTOR (SEQ ID NO:13).

Variants of BMSOTR polypeptide are also encompassed by the present invention, such as a BMSOTR variant polypeptide comprising the amino acid sequence of SEQ ID NO:4 as shown in FIGS. 2A-B and the BMSOTR variant nucleic acid (SEQ ID NO:3; FIGS. 2A-B) which encodes SEQ ID NO:4. Preferably, a BMSOTR variant has at least 75 to 80%, more preferably at least 85 to 90%, and even more preferably at least 90% amino acid sequence identity to the BMSOTR amino acid sequence disclosed herein, and more preferably, retains at least one biological, immunological, or other functional characteristic or activity of the non-variant BMSOTR polypeptide. Most preferred are BMSOTR variants or substantially purified fragments thereof having at least 95% amino acid sequence identity to that of SEQ ID NO:2. Variants of BMSOTR polypeptide or substantially purified fragments of the polypeptide can also include amino acid sequences that differ from the SEQ ID NO:2 amino acid sequence only by conservative substitutions. The invention also encompasses polypeptide homologues of the amino acid sequence as set forth in SEQ ID NO:2.

The strong homology to oxytocin receptors, combined with the predominate expression in pancreas tissue suggests a potential utility for BMSOTR polynucleotides and polypeptides in treating, diagnosing, prognosing, and/or preventing pancreatic, in addition to metabolic and gastrointestinal disorders.

In preferred embodiments, BMSOTR polynucleotides and polypeptides including agonists, antagonists, and fragments thereof, have uses which include treating, diagnosing, prognosing, and/or preventing the following, non-limiting, diseases or disorders of the pancreas: diabetes mellitus, diabetes, type 1 diabetes, type 2 diabetes, adult onset diabetes, indications related to islet cell transplantation, indications related to pancreatic transplantation, pancreatitis, pancreatic cancer, pancreatic exocrine insufficiency, alcohol induced pancreatitis, maldigestion of fat, maldigestion of protein, hypertriglyceridemia, vitamin b12 malabsorption, hypercalcemia, hypocalcemia, hyperglycemia, ascites, pleural effusions, abdominal pain, pancreatic necrosis, pancreatic abscess, pancreatic pseudocyst, gastrinomas, pancreatic islet cell hyperplasia, multiple endocrine neoplasia type 1 (men 1) syndrome, insulitis, amputations, diabetic neuropathy, pancreatic auto-immune disease, genetic defects of cell function, HNF-1 aberrations (formerly MODY3), glucokinase aberrations (formerly MODY2), HNF-4 aberrations (formerly MODY1), mitochondrial DNA aberrations, genetic defects in insulin action, type a insulin resistance, leprechaunism, Rabson-Mendenhall syndrome, lipoatrophic diabetes, pancreatectomy, cystic fibrosis, hemochromatosis, fibrocalculous pancreatopathy, endocrinopathies, acromegaly, Cushing's syndrome, glucagonoma, pheochromocytoma, hyperthyroidism, somatostatinoma, aldosteronoma, drug- or chemical-induced diabetes such as from the following drugs: Vacor, Pentamdine, Nicotinic acid, Glucocorticoids, Thyroid hormone, Diazoxide, Adrenergic agonists, Thiazides, Dilantin, and Interferon, pancreatic infections, congential rubella, cytomegalovirus, uncommon forms of immune-mediated diabetes, “stiff-man” syndrome, anti-insulin receptor antibodies, in addition to other genetic syndromes sometimes associated with diabetes which include, for example, Down's syndrome, Klinefelter's syndrome, Turner's syndrome, Wolfram's syndrome, Friedrich's ataxia, Huntington's chorea, Lawrence Moon Beidel syndrome, Myotonic dystrophy, Porphyria, and Prader Willi syndrome, and/or Gestational diabetes mellitus (GDM).

Alternatively, the strong homology to oxytocin receptors, combined with the predominate expression in brain tissue suggests a potential utility for BMSOTR polynucleotides and polypeptides in treating, diagnosing, prognosing, and/or preventing neurodegenerative disease states, behavioral disorders, or inflammatory conditions. Briefly, the uses include, but are not limited to the detection, treatment, and/or prevention of Alzheimer's Disease, Parkinson's Disease, Huntington's Disease, Tourette Syndrome, meningitis, encephalitis, demyelinating diseases, peripheral neuropathies, neoplasia, trauma, congenital malformations, spinal cord injuries, ischemia and infarction, aneurysms, hemorrhages, schizophrenia, mania, dementia, paranoia, obsessive compulsive disorder, depression, panic disorder, learning disabilities, ALS, psychoses, autism, and altered behaviors, including disorders in feeding, sleep patterns, balance, and perception. In addition, elevated expression of this gene product in regions of the brain indicates it plays a role in normal neural function. Potentially, this gene product is involved in synapse formation, neurotransmission, learning, cognition, homeostasis, or neuronal differentiation or survival. Furthermore, the protein may also be used to determine biological activity, to raise antibodies, as tissue markers, to isolate cognate ligands or receptors, to identify agents that modulate their interactions, in addition to its use as a nutritional supplement. Protein, as well as, antibodies directed against the protein may show utility as a tumor marker and/or immunotherapy targets for the above listed tissues.

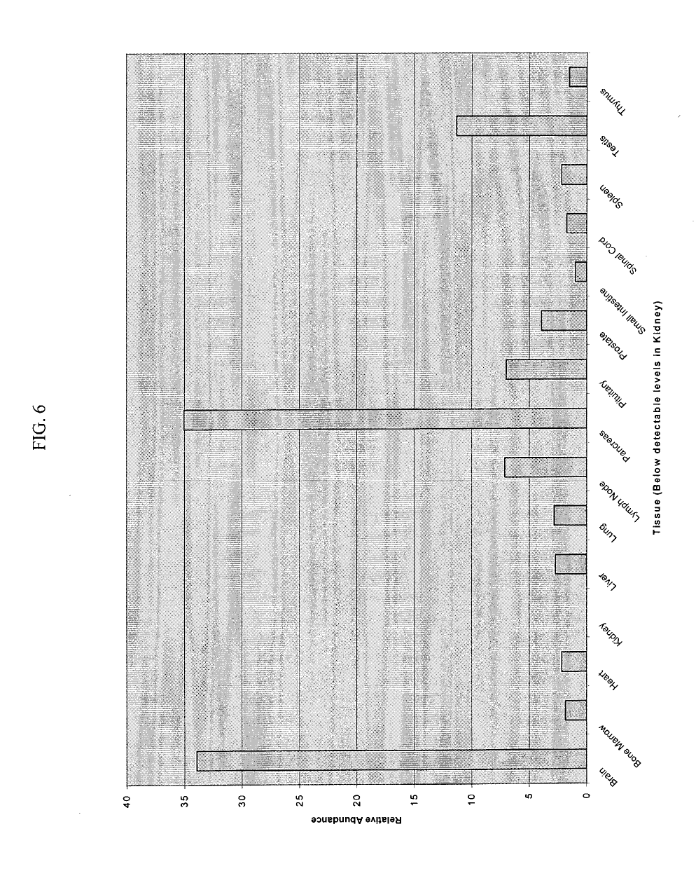

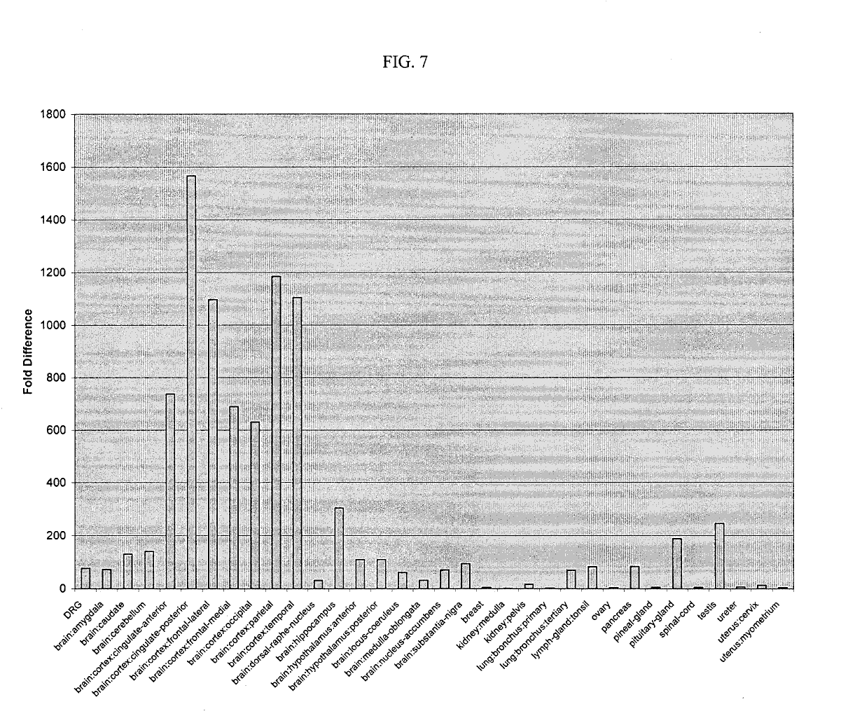

Expanded analysis of BMSOTR expression levels by TaqMan™ quantitative PCR (see FIG. 7) confirmed the initial SYBR green expression analysis of the BMSOTR polypeptide by demonstrating that BMSOTR is expressed in the brain (FIG. 6). BMSOTR mRNA was expressed predominately in the nervous system, specifically the cortex, hippocampus, and to a lesser extent in other brain sub-regions. Expression through out the female reproductive tract was not observed, especially in the uterus myometrium. Extremely small amounts of BMSOTR transcripts are found in the DRG, but are not observed in the spinal cord.

Collectively the expression data suggest a role for BMSOTR in a diverse set of neural processes, including executive functions concerned with the organization of behavior, memory and cognitive functioning. BMSOTR expression in the dorsal raphe, the site of origin of the serotonin nervous system, suggests that this GPCR could participate in the control of anxiety, fear, depression, sleep and pain. Expression in the locus coeruleus suggests involvement in the maintenance of an attentive or alert state. Expression in the nucleus accumbens, the region of the brain best known as the ‘reward center’ effecting the release of neurotransmitters such as dopamine, opioid peptides, serotonin, GABA, and glutamate suggests a possible role in the establishment of addictive behaviors. Expression in the hypothalamus suggest a possible involvement the control of a diverse set of homeostatic and neuroendocrine functions, while expression in the hippocampus suggest a role in the establishment of long term potentiation. Expression in the pineal gland suggests a possible involvement in the establishment and maintenance of circadian rhythms and the control of the sleep/wake cycle. Expression in the substantia nigra suggests a possible involvement with the dopaminergic functions that emanate from this region. Expression in the DRG suggest roles in various neuronal transmission systems, most notably pain.

In another embodiment, the present invention encompasses polynucleotides which encode BMSOTR polypeptides. Accordingly, any nucleic acid sequence that encodes the amino acid sequence of a BMSOTR polypeptide of the invention can be used to produce recombinant molecules that express a BMSOTR protein. More particularly, the invention encompasses the BMSOTR polynucleotide having the nucleic acid sequence of SEQ ID NO:1. The present invention also provides a clone containing BMSOTR cDNA, deposited at the American Type Culture Collection (ATCC), 10801 University Boulevard, Manassas, Va. 20110-2209 on ______ and under ATCC Accession No(s). ______ according to the terms of the Budapest Treaty.

As will be appreciated by the skilled practitioner in the art, the degeneracy of the genetic code results in many nucleotide sequences that can encode the described BMSOTR polypeptide. Some of the sequences bear minimal or no homology to the nucleotide sequences of any known and naturally occurring gene. Accordingly, the present invention contemplates each and every possible variation of nucleotide sequence that could be made by selecting combinations based on possible codon choices. These combinations are made in accordance with the standard triplet genetic code as applied to the nucleotide sequence of naturally occurring BMSOTR, and all such variations are to be considered as being specifically disclosed and able to be understood by the skilled practitioner.

In preferred embodiments, the following N-terminal BMSOTR deletion polypeptides are encompassed by the present invention: M1-F383, E2-F383, D3-F383, L4-F383, F5-F383, S6-F383, P7-F383, S8-F383, I9-F383, L10-F383, P11-F383, P12-F383, A13-F383, P14-F383, N15-F383, I16-F383, S17-F383, V18-F383, P19-F383, I20-F383, L21-F383, L22-F383, G23-F383, W24-F383, G25-F383, L26-F383, N27-F383, L28-F383, T29-F383, L30-F383, G31-F383, Q32-F383, G33-F383, A34-F383, P35-F383, A36-F383, S37-F383, G38-F383, P39-F383, P40-F383, S41-F383, R42-F383, R43-F383, V44-F383, R45-F383, L46-F383, V47-F383, F48-F383, L49-F383, G50-F383, V51-F383, I52-F383, L53-F383, V54-F383, V55-F383, A56-F383, V57-F383, A58-F383, G59-F383, N60-F383, T61-F383, T62-F383, V63-F383, L64-F383, C65-F383, R66-F383, L67-F383, C68-F383, G69-F383, G70-F383, G71-F383, G72-F383, P73-F383, W74-F383, A75-F383, G76-F383, P77-F383, K78-F383, R79-F383, R80-F383, K81-F383, M82-F383, D83-F383, F84-F383, L85-F383, L86-F383, V87-F383, Q88-F383, L89-F383, A90-F383, L91-F383, A92-F383, D93-F383, L94-F383, Y95-F383, A96-F383, C97-F383, G98-F383, G99-F383, T100-F383, A101-F383, L102-F383, S103-F383, Q104-F383, L105-F383, A106-F383, W107-F383, E108-F383, L109-F383, L110-F383, G111-F383, E112-F383, P113-F383, R114-F383, A115-F383, A161-F383, T117-F383, G118-F383, D119-F383, L120-F383, A121-F383, C122-F383, R123-F383, F124-F383, L125-F383, Q126-F383, L127-F383, L128-F383, Q129-F383, A130-F383, S131-F383, G132-F383, R133-F383, G134-F383, A135-F383, S136-F383, A137-F383, H138-F383, L139-F383, V140-F383, V141-F383, L142-F383, I143-F383, A144-F383, L145-F383, E146-F383, R147-F383, R148-F383, R149-F383, A150-F383, P151-F383, G152-F383, A153-F383, P154-F383, L155-F383, S156-F383, A157-F383, R158-F383, A159-F383, W160-F383, P161-F383, G162-F383, E163-F383, R164-F383, R165-F383, C166-F383, H167-F383, G168-F383, I169-F383, F170-F383, A171-F383, P172-F383, L173-F383, P174-F383, R175-F383, W176-F383, H177-F383, L178-F383, Q179-F383, V180-F383, Y181-F383, A182-F383, F183-F383, Y184-F383, E185-F383, A186-F383, V187-F383, A188-F383, G189-F383, F190-F383, V191-F383, A192-F383, P193-F383, V194-F383, T195-F383, V196-F383, L197-F383, G198-F383, V199-F383, A200-F383, C201-F383, G202-F383, H203-F383, L204-F383, L205-F383, S206-F383, V207-F383, W208-F383, W209-F383, R210-F383, H211-F383, R212-F383, P213-F383, Q214-F383, A215-F383, P216-F383, A217-F383, A218-F383, A219-F383, A220-F383, P221-F383, W222-F383, S223-F383, A224-F383, S225-F383, P226-F383, G227-F383, R228-F383, A229-F383, P230-F383, A231-F383, P232-F383, S233-F383, A234-F383, L235-F383, P236-F383, R237-F383, A238-F383, K239-F383, V240-F383, Q241-F383, S242-F383, L243-F383, K244-F383, M245-F383, S246-F383, L247-F383, L248-F383, L249-F383, A250-F383, L251-F383, L252-F383, F253-F383, V254-F383, G255-F383, C256-F383, E257-F383, L258-F383, P259-F383, Y260-F383, F261-F383, A262-F383, A263-F383, R264-F383, L265-F383, A266-F383, A267-F383, A268-F383, W269-F383, S270-F383, S271-F383, G272-F383, P273-F383, A274-F383, G275-F383, D276-F383, W277-F383, E278-F383, G279-F383, E280-F383, G281-F383, L282-F383, S283-F383, A284-F383, A285-F383, L286-F383, R287-F383, V288-F383, V289-F383, A290-F383, M291-F383, A292-F383, N293-F383, S294-F383, A295-F383, L296-F383, N297-F383, P298-F383, F299-F383, V300-F383, Y301-F383, L302-F383, F303-F383, F304-F383, Q305-F383, A306-F383, G307-F383, D308-F383, C309-F383, R310-F383, L311-F383, R312-F383, R313-F383, Q314-F383, L315-F383, R316-F383, K317-F383, R318-F383, L319-F383, G320-F383, S321-F383, L322-F383, C323-F383, C324-F383, A325-F383, P326-F383, Q327-F383, G328-F383, G329-F383, A330-F383, E331-F383, D332-F383, E333-F383, E334-F383, G335-F383, P336-F383, R337-F383, G338-F383, H339-F383, Q340-F383, A341-F383, L342-F383, Y343-F383, R344-F383, Q345-F383, R346-F383, W347-F383, P348-F383, H349-F383, P350-F383, H351-F383, Y352-F383, H353-F383, H354-F383, A355-F383, R356-F383, R357-F383, E358-F383, P359-F383, L360-F383, D361-F383, E362-F383, G363-F383, G364-F383, L365-F383, R366-F383, P367-F383, P368-F383, P369-F383, P370-F383, R371-F383, P372-F383, R373-F383, P374-F383, L375-F383, P376-F383, and/or C377-F383 of SEQ ID NO:2. Polynucleotide sequences encoding these polypeptides are also provided. The present invention also encompasses the use of these N-terminal BMSOTR deletion polypeptides as immunogenic and/or antigenic epitopes as described elsewhere herein.

In preferred embodiments, the following C-terminal BMSOTR deletion polypeptides are encompassed by the present invention: M1-F383, M1-A382, M1-S381, M1-E380, M1-C379, M1-S378, M1-C377, M1-P376, M1-L375, M1-P374, M1-R373, M1-P372, M1-R371, M1-P370, M1-P369, M1-P368, M1-P367, M1-R366, M1-L365, M1-G364, M1-G363, M1-E362, M1-D361, M1-L360, M1-P359, M1-E358, M1-R357, M1-R356, M1-A355, M1-H354, M1-H353, M1-Y352, M1-H351, M1-P350, M1-H349, M1-P348, M1-W347, M1-R346, M1-Q345, M1-R344, M1-Y343, M1-L342, M1-A341, M1-Q340, M1-H339, M1-G338, M1-R337, M1-P336, M1-G335, M1-E334, M1-E333, M1-D332, M1-E331, M1-A330, M1-G329, M1-G328, M1-Q327, M1-P326, M1-A325, M1-C324, M1-C323, M1-L322, M1-S321, M1-G320, M1-L319, M1-R318, M1-K317, M1-R316, M1-L315, M1-Q314, M1-R313, M1-R312, M1-L311, M1-R310, M1-C309, M1-D308, M1-G307, M1-A306, M1-Q305, M1-F304, M1-F303, M1-L302, M1-Y301, M1-V300, M1-F299, M1-P298, M1-N297, M1-L296, M1-A295, M1-S294, M1-N293, M1-A292, M1-M291, M1-A290, M1-V289, M1-V288, M1-R287, M1-L286, M1-A285, M1-A284, M1-S283, M1-L282, M1-G281, M1-E280, M1-G279, M1-E278, M1-W277, M1-D276, M1-G275, M1-A274, M1-P273, M1-G272, M1-S271, M1-S270, M1-W269, M1-A268, M1-A267, M1-A266, M1-L265, M1-R264, M1-A263, M1-A262, M1-F261, M1-Y260, M1-P259, M1-L258, M1-E257, M1-C256, M1-G255, M1-V254, M1-F253, M1-L252, M1-L251, M1-A250, M1-L249, M1-L248, M1-L247, M1-S246, M1-M245, M1-K244, M1-L243, M1-S242, M1-Q241, M1-V240, M1-K239, M1-A238, M1-R237, M1-P236, M1-L235, M1-A234, M1-S233, M1-P232, M1-A231, M1-P230, M1-A229, M1-R228, M1-G227, M1-P226, M1-S225, M1-A224, M1-S223, M1-W222, M1-P221, M1-A220, M1-A219, M1-A218, M1-A217, M1-P216, M1-A215, M1-Q214, M1-P213, M1-R212, M1-H211, M1-R210, M1-W209, M1-W208, M1-V207, M1-S206, M1-L205, M1-L204, M1-H203, M1-G202, M1-C201, M1-A200, M1-V199, M1-G198, M1-L197, M1-V196, M1-T195, M1-V194, M1-P193, M1-A192, M1-V191, M1-F190, M1-G189, M1-A188, M1-V187, M1-A186, M1-E185, M1-Y184, M1-F183, M1-A182, M1-Y181, M1-V180, M1-Q179, M1-L178, M1-H177, M1-W176, M1-R175, M1-P174, M1-L173, M1-P172, M1-A171, M1-F170, M1-I169, M1-G168, M1-H167, M1-C166, M1-R165, M1-R164, M1-E163, M1-G162, M1-P161, M1-W160, M1-A159, M1-R158, M1-A157, M1-S156, M1-L155, M1-P154, M1-A153, M1-G152, M1-P151, M1-A150, M1-R149, M1-R148, M1-R147, M1-E146, M1-L145, M1-A144, M1-I143, M1-L142, M1-V141, M1-V140, M1-L139, M1-H138, M1-A137, M1-S136, M1-A135, M1-G134, M1-R133, M1-G132, M1-S131, M1-A130, M1-Q129, M1-L128, M1-L127, M1-Q126, M1-L125, M1-F124, M1-R123, M1-C122, M1-A121, M1-L120, M1-D119, M1-G118, M1-T117, M1-A116, M1-A115, M1-R114, M1-P113, M1-E112, M1-G111, M1-L110, M1-L109, M1-E108, M1-W107, M1-A106, M1-L105, M1-Q104, M1-S103, M1-L102, M1-A111, M1-T110, M1-G99, M1-G98, M1-C97, M1-A96, M1-Y95, M1-L94, M1-D93, M1-A92, M1-L91, M1-A90, M1-L89, M1-Q88, M1-V87, M1-L86, M1-L85, M1-F84, M1-D83, M1-M82, M1-K81, M1-R80, M1-R79, M1-K178, M1-P77, M1-G76, M1-A75, M1-W74, M1-P73, M1-G72, M1-G71, M1-G70, M1-G69, M1-C68, M1-L67, M1-R66, M1-C65, M1-L64, M1-V63, M1-T62, M1-T61, M1-N60, M1-G59, M1-A58, M1-V57, M1-A56, M1-V55, M1-V54, M1-L53, M1-I52, M1-V51, M1-G50, M1-L49, M1-F48, M1-V47, M1-I46, M1-R45, M1-V44, M1-R43, M1-R42, M1-S41, M1-P40, M1-P39, M1-G38, M1-S37, M1-A36, M1-P35, M1-A34, M1-G33, M1-Q32, M1-G31, M1-L30, M1-T29, M1-L28, M1-N27, M1-L26, M1-G25, M1-W24, M1-G23, M1-L22, M1-L21, M-1-I20, M1-P19, M1-V18, M1-S17, M1-I16, M1-N15, M1-P14, M1-A13, M1-P12, M1-P11, M1-L10, M1-I9, M1-S8, and/or M1-P7 of SEQ ID NO:2. Polynucleotide sequences encoding these polypeptides are also provided. The present invention also encompasses the use of these C-terminal BMSOTR deletion polypeptides as immunogenic and/or antigenic epitopes as described elsewhere herein.

Alternatively, preferred polypeptides of the present invention encompass polypeptide sequences corresponding to, for example, internal regions of the BMSOTR polypeptide (e.g., any combination of both N- and C-terminal BMSOTR polypeptide deletions) of SEQ ID NO:2. For example, internal regions could be defined by the equation: amino acid NX to amino acid CX, wherein NX refers to any N-terminal deletion polypeptide amino acid of BMSOTR (SEQ ID NO:2), and where CX refers to any C-terminal deletion polypeptide amino acid of BMSOTR (SEQ ID NO:2). Polynucleotides encoding these polypeptides are also provided. The present invention also encompasses the use of these polypeptides as an immunogenic and/or antigenic epitope as describes elsewhere herein.

In another embodiment, the present invention encompasses a polynucleotide lacking the initiating start codon, in addition to, the resulting encoded polypeptide of BMSOTR. Specifically, the present invention encompasses the polynucleotide corresponding to nucleotides 119 through 1264 of SEQ ID NO:1, and the polypeptide corresponding to amino acids 2 through 383 of SEQ ID NO:2. Also encompassed are recombinant vectors comprising the encoding sequence, and host cells comprising the vector.

In preferred embodiments, the following N-terminal BMSOTR variant deletion polypeptides are encompassed by the present invention: M1-F434, E2-F434, D3-F434, L4-F434, F5-F434, S6-F434, P7-F434, S8-F434, 19-F434, L10-F434, P11-F434, P12-F434, A13-F434, P14-F434, N15-F434, I16-F434, S17-F434, V18-F434, P19-F434, 120-F434, L21-F434, L22-F434, G23-F434, W24-F434, G25-F434, L26-F434, N27-F434, L28-F434, T29-F434, L30-F434, G31-F434, Q32-F434, G33-F434, A34-F434, P35-F434, A36-F434, S37-F434, G38-F434, P39-F434, P40-F434, S41-F434, R42-F434, R43-F434, V44-F434, R45-F434, L46-F434, V47-F434, F48-F434, L49-F434, G50-F434, V51-F434, 152-F434, L53-F434, V54-F434, V55-F434, A56-F434, V57-F434, A58-F434, G59-F434, N60-F434, T61-F434, T62-F434, V63-F434, L64-F434, C65-F434, R66-F434, L67-F434, C68-F434, G69-F434, G70-F434, G71-F434, G72-F434, P73-F434, W74-F434, A75-F434, G76-F434, P77-F434, K78-F434, R79-F434, R80-F434, K81-F434, M82-F434, D83-F434, F84-F434, L85-F434, L86-F434, V87-F434, Q88-F434, L89-F434, A90-F434, L91-F434, A92-F434, D93-F434, L94-F434, Y95-F434, A96-F434, C97-F434, G98-F434, G99-F434, T100-F434, A101-F434, L102-F434, S103-F434, Q104-F434, L105-F434, A106-F434, W107-F434, E108-F434, L109-F434, L110-F434, G1′-F434, E112-F434, P113-F434, R114-F434, A115-F434, A116-F434, Ti 17-F434, G118-F434, D119-F434, L120-F434, A121-F434, C122-F434, R123-F434, F124-F434, L125-F434, Q126-F434, L127-F434, L128-F434, Q129-F434, A130-F434, S131-F434, G132-F434, R133-F434, G134-F434, A135-F434, S136-F434, A137-F434, H138-F434, L139-F434, V140-F434, V141-F434, L142-F434, I143-F434, A144-F434, L145-F434, E146-F434, R147-F434, R148-F434, R149-F434, A150-F434, V151-F434, R152-F434, L153-F434, P154-F434, H155-F434, G156-F434, R157-F434, P158-F434, L159-F434, P160-F434, A161-F434, R162-F434, A163-F434, L164-F434, A165-F434, A166-F434, L167-F434, G168-F434, W169-F434, L170-F434, L171-F434, A172-F434, L173-F434, L174-F434, L175-F434, A176-F434, L177-F434, P178-F434, P179-F434, A180-F434, F181-F434, V182-F434, V183-F434, R184-F434, G185-F434, D186-F434, S187-F434, P188-F434, S189-F434, P190-F434, L191-F434, P192-F434, P193-F434, P194-F434, P195-F434, P196-F434, P197-F434, T198-F434, S199-F434, L200-F434, Q201-F434, P202-F434, G203-F434, A204-F434, P205-F434, and/or P206-F434 of SEQ ID NO:4. Polynucleotide sequences encoding these polypeptides are also provided. The present invention also encompasses the use of these N-terminal BMSOTR variant deletion polypeptides as immunogenic and/or antigenic epitopes as described elsewhere herein.

In preferred embodiments, the following C-terminal BMSOTR variant deletion polypeptides are encompassed by the present invention: M1-F434, M1-A433, M1-S432, M1-E431, M1-C430, M1-S429, M1-C428, M1-P427, M1-L426, M1-P425, M1-R424, M1-P423, M1-R422, M1-P421, M1-P420, M1-P419, M1-P418, M1-R417, M1-L416, M1-G415, M1-G414, M1-E413, M1-D412, M1-L411, M1-P410, M1-E409, M1-R408, M1-R407, M1-A406, M1-H405, M1-H404, M1-Y403, M1-H402, M1-P401, M1-H400, M1-P399, M1-W398, M1-R397, M1-Q396, M1-R395, M1-Y394, M1-L393, M1-A392, M1-Q391, M1-H390, M1-G389, M1-R388, M1-P387, M1-G386, M1-E385, M1-E384, M1-D383, M1-E382, M1-A381, M1-G380, M1-G379, M1-Q378, M1-P377, M1-A376, M1-C375, M1-C374, M1-L373, M1-S372, M1-G371, M1-L370, M1-R369, M1-K368, M1-R367, M1-L366, M1-Q365, M1-R364, M1-R363, M1-L362, M1-R361, M1-C360, M1-D359, M1-G358, M1-A357, M1-Q356, M1-F355, M1-F354, M1-L353, M1-Y352, M1-V351, M1-F350, M1-P349, M1-N348, M1-L347, M1-A346, M1-S345, M1-N344, M1-A343, M1-M342, M1-A341, M1-V340, M1-V339, M1-R338, M1-L337, M1-A336, M1-A335, M1-S334, M1-L333, M1-G332, M1-E331, M1-G330, M1-E329, M1-W328, M1-D327, M1-G326, M1-A325, M1-P324, M1-G323, M1-S322, M1-S321, M1-W320, M1-A319, M1-A318, M1-A317, M1-L316, M1-R315, M1-A314, M1-A313, M1-F312, M1-Y311, M1-P310, M1-L309, M1-E308, M1-C307, M1-G306, M1-V305, M1-F304, M1-L303, M1-L302, M1-A301, M1-L300, M1-L299, M1-L298, M1-S297, M1-M296, M1-K295, M1-L294, M1-S293, M1-Q292, M1-V291, M1-K290, M1-A289, M1-R288, M1-P287, M1-L286, M1-A285, M1-S284, M1-P283, M1-A282, M1-P281, M1-A280, M1-R279, M1-G278, M1-P277, M1-S276, M1-A275, M1-S274, M1-W273, M1-P272, M1-A271, M1-A270, M1-A269, M1-A268, M1-P267, M1-A266, M1-Q265, M1-P264, M1-R263, M1-H262, M1-R261, M1-W260, M1-W259, M1-V258, M1-S257, M1-L256, M1-L255, M1-H254, M1-G253, M1-C252, M1-A251, M1-V250, M1-G249, M1-L248, M1-V247, M1-T246, M1-V245, M1-P244, M1-A243, M1-V242, M1-F241, M1-G240, M1-A239, M1-V238, M1-A237, M1-E236, M1-Y235, M1-F234, M1-A233, M1-Y232, M1-V231, M1-Q230, M1-L229, M1-H228, M1-W227, M1-R226, M1-P225, M1-L224, M1-P223, M1-A222, M1-F221, M1-I220, M1-G219, M1-H218, M1-C217, M1-R216, M1-R215, M1-E214, M1-G213, M1-P212, M1-W211, M1-A210, M1-R209, M1-A208, M1-A207, M1-P206, M1-P205, M1-A204, M1-G203, M1-P202, M1-Q201, M1-L200, M1-S199, M1-T198, M1-P197, M1-P196, M1-P195, M1-P194, M1-P193, M1-P192, M1-L191, M1-P190, M1-S189, M1-P188, M1-S187, M1-D186, M1-G185, M1-R184, M1-V183, M1-V182, M1-F181, M1-A180, M1-P179, M1-P178, M1-L177, M1-A176, M1-L175, M1-L174, M1-L173, M1-A172, M1-L171, M1-L170, M1-W169, M1-G168, M1-L167, M1-A166, M1-A165, M1-L164, M1-A163, M1-R162, M1-A161, M1-P160, M1-L159, M1-P158, M1-R157, M1-G156, M1-H155, M1-P154, M1-L153, M1-R152, and/or M1-V151 of SEQ ID NO:4. Polynucleotide sequences encoding these polypeptides are also provided. The present invention also encompasses the use of these C-terminal BMSOTR variant deletion polypeptides as immunogenic and/or antigenic epitopes as described elsewhere herein.

Alternatively, preferred polypeptides of the present invention encompass polypeptide sequences corresponding to, for example, internal regions of the BMSOTR variant polypeptide (e.g., any combination of both N- and C-terminal BMSOTR polypeptide deletions) of SEQ ID NO:4. For example, internal regions could be defined by the equation: amino acid NX to amino acid CX, wherein NX refers to any N-terminal deletion polypeptide amino acid of the BMSOTR variant (SEQ ID NO:4), and where CX refers to any C-terminal deletion polypeptide amino acid of the BMSOTR variant (SEQ ID NO:4). Polynucleotides encoding these polypeptides are also provided. The present invention also encompasses the use of these polypeptides as an immunogenic and/or antigenic epitope as describes elsewhere herein.

In another embodiment, the present invention encompasses a polynucleotide lacking the initiating start codon, in addition to, the resulting encoded polypeptide of the BMSOTR variant. Specifically, the present invention encompasses the polynucleotide corresponding to nucleotides 119 through 1417 of SEQ ID NO:3, and the polypeptide corresponding to amino acids 2 through 434 of SEQ ID NO:4. Also encompassed are recombinant vectors comprising the encoding sequence, and host cells comprising the vector.

In one embodiment, a BMSOTR polypeptide comprises a portion of the amino sequence depicted in FIGS. 1A-B. In another embodiment, a BMSOTR polypeptide comprises at least 4, 5, 6, 7, 8, 9, 10, 11, 12, 13, 14, 15, 16, 17, 18, 19 or 20 amino acids of the amino sequence depicted in FIGS. 1A-B. In further embodiments, the following BMSOTR polypeptide fragments are specifically excluded from the present invention:

(SEQ ID NO:56)

AAAWSSGPAGDWEGEGLSAALRVVAMANSALNPFVYLFFQAGDCRLRRQL
RKRLGSLCCAPQGGAEDEEGPRGHQALYRQRWPHPHYHHARREPLDEGGL
RPPPPRPRPLPCSCESAF;

(SEQ ID NO:57)

DWEGEGLSAALRVVAMANSALNPFVYLFFQAGDCRLRRQLRKRLGSLCCA
PQGGAEDEEGPRGHQALYRQRWPHPHYHHARREPLDEGGLRPPPPRPRPL
PCSCESAF;

(SEQ ID NO:58)

AGDWEGEGLSAALRVVAMANSALNPFVYLFFQAGDCRLRRQLRKLGSLCC
APQGGAEDEEGPRGHQALYRQRWPHPHYHHARREPLDEGGLRPPPPRPRP
LPCSCESAF;

(SEQ ID NO:59)

MANSALNPFVYLFFQAGDCRLRRQLRKRLGSLCCAPQGGAEDEEGPRGHQ
ALYRQRWPHPHYHHARREP;

(SEQ ID NO:60)

PLDEGGLRPPPPRPRPLPCSCESAF;

(SEQ ID NO:61)

AAAWSSGPAGDWEGEGLSAALRVVAMANSALNPFVYLFFQAGDCRLRRQL
RKRLGSLCCAPQGGAEDEEGPRGHQALYRQR;

(SEQ ID NO:62)

PHPHYHHARREPLDEGGLRPPPPRPRPLPCSCESAF;

(SEQ ID NO:63)

MEDLFSPSILPPAPNISVPILLGWGLNLTLGQGAPASGPPSRRVRLVFLG
VWVVAVAGNTTVLCRL;

(SEQ ID NO:64)

EGLSAALRVVAMANSALNPFVYLFFQAGDCRLRRQLRKRLGSLCCAP;

(SEQ ID NO:65)

and/or VAMANSALNPFVYLFFQAGDC.

Many polynucleotide sequences, such as EST sequences, are publicly available and accessible through sequence databases. Some of these sequences are related to SEQ ID NO:1 and may have been publicly available prior to conception of the present invention. Preferably, such related polynucleotides are specifically excluded from the scope of the present invention. To list every related sequence would be cumbersome. Accordingly, preferably excluded from the present invention are one or more polynucleotides consisting of a nucleotide sequence described by the general formula of a-b, where a is any integer between 1 to 1826 of SEQ ID NO:1, b is an integer between 15 to 1840, where both a and b correspond to the positions of nucleotide residues shown in SEQ ID NO:1, and where b is greater than or equal to a+14.

TABLE II

| | | ATCC | | NT | Total | 5’ NT | 3’ | AA | Total |
|---|---|---|---|---|---|---|---|---|---|
| | CDNA | Deposit | | SEQ | NT | of Start | NT | Seq | AA |
| Gene | Clone | No. Z | | ID. No. | Seq of | Codon | of | ID | of |
| No. | ID | and Date | Vector | X | Clone | of ORF | ORF | No. Y | ORF |
| 1. | BMSOTR | N/A | pSport | 1 | 1840 | 116 | 1264 | 2 | 383 |
| | (also referred | | 1 | | | | | | |
| | to as GPCR- | | | | | | | | |
| | 170 and/or | | | | | | | | |
| | OXYTOCIN_{—} | | | | | | | | |
| | CAND) | | | | | | | | |
| 2. | BMSOTR | N/A | pSport | 3 | 1993 | 116 | 1417 | 4 | 434 |
| | variant | | 1 | | | | | | |

Table II summarizes the information corresponding to each “Gene No.” described above. The nucleotide sequence identified as “NT SEQ ID NO:1 and/or 3” was assembled from partially homologous (“overlapping”) sequences obtained from the “cDNA clone ID” identified in Table II and, in some cases, from additional related DNA clones. The overlapping sequences were assembled into a single contiguous sequence of high redundancy (usually several overlapping sequences at each nucleotide position), resulting in a final sequence identified as SEQ ID NO:1 and/or 3. However, for the purposes of the present invention, SEQ ID NO:1 and/or 3 may refer to any polynucleotide of the present invention.

The cDNA Clone ID was deposited on the date and given the corresponding deposit number listed in “ATCC Deposit No:Z and Date.” “Vector” refers to the type of vector contained in the cDNA Clone ID.

“Total NT Seq. Of Clone” refers to the total number of nucleotides in the clone contig identified by “Gene No.” The deposited clone may contain all or most of the sequence of SEQ ID NO:1 and/or 3. The nucleotide position of SEQ ID NO:1 and/or 3 of the putative start codon (methionine) is identified as “5′ NT of Start Codon of ORF.”

The translated amino acid sequence, beginning with the methionine, is identified as “AA SEQ ID NO:2 and/or 4” although other reading frames can also be easily translated using known molecular biology techniques. The polypeptides produced by these alternative open reading frames are specifically contemplated by the present invention.

The total number of amino acids within the open reading frame of SEQ ID NO:2 and/or 4 is identified as “Total AA of ORF”.

SEQ ID NO:1 and/or 3 (where X may be any of the polynucleotide sequences disclosed in the sequence listing) and the translated SEQ ID NO:2 and/or 4 (where Y may be any of the polypeptide sequences disclosed in the sequence listing) are sufficiently accurate and otherwise suitable for a variety of uses well known in the art and described further herein. For instance, SEQ ID NO:1 and/or 3 is useful for designing nucleic acid hybridization probes that will detect nucleic acid sequences contained in SEQ ID NO:1 and/or 3 or the cDNA contained in the deposited clone. These probes will also hybridize to nucleic acid molecules in biological samples, thereby enabling a variety of forensic and diagnostic methods of the invention. Similarly, polypeptides identified from SEQ ID NO:2 and/or 4 may be used, for example, to generate antibodies which bind specifically to proteins containing the polypeptides and the proteins encoded by the cDNA clones identified in Table II.

Nevertheless, DNA sequences generated by sequencing reactions can contain sequencing errors. The errors exist as misidentified nucleotides, or as insertions or deletions of nucleotides in the generated DNA sequence. The erroneously inserted or deleted nucleotides may cause frame shifts in the reading frames of the predicted amino acid sequence. In these cases, the predicted amino acid sequence diverges from the actual amino acid sequence, even though the generated DNA sequence may be greater than 99.9% identical to the actual DNA sequence (for example, one base insertion or deletion in an open reading frame of over 1000 bases).

Accordingly, for those applications requiring precision in the nucleotide sequence or the amino acid sequence, the present invention provides not only the generated nucleotide sequence identified as SEQ ID NO:1 and the predicted translated amino acid sequence identified as SEQ ID NO:2, but also a sample of plasmid DNA containing a cDNA of the invention deposited with the ATCC, as set forth in Table II. The nucleotide sequence of each deposited clone can readily be determined by sequencing the deposited clone in accordance with known methods. The predicted amino acid sequence can then be verified from such deposits. Moreover, the amino acid sequence of the protein encoded by a particular clone can also be directly determined by peptide sequencing or by expressing the protein in a suitable host cell containing the deposited cDNA, collecting the protein, and determining its sequence.

Although nucleic acid sequences which encode the BMSOTR polypeptide and variants thereof are preferably capable of hybridizing to the nucleotide sequence of the naturally occurring BMSOTR polypeptide under appropriately selected conditions of stringency, it may be advantageous to produce nucleotide sequences encoding BMSOTR polypeptides, or derivatives thereof, which possess a substantially different codon usage. For example, codons may be selected to increase the rate at which expression of the peptide/polypeptide occurs in a particular prokaryotic or eukaryotic host in accordance with the frequency with which particular codons are utilized by the host. Another reason for substantially altering the nucleotide sequence encoding a BMSOTR polypeptide, or its derivatives, without altering the encoded amino acid sequences, includes the production of RNA transcripts having more desirable properties, such as a greater half-life, than transcripts produced from the naturally occurring sequence.

The present invention also encompasses the production of DNA sequences, or portions thereof, which encode the BMSOTR polypeptide, or derivatives thereof, entirely by synthetic chemistry. After production, the synthetic sequence may be inserted into any of the many available expression vectors and cell systems using reagents that are well known and practiced by those in the art. Moreover, synthetic chemistry may be used to introduce mutations into a sequence encoding a BMSOTR polypeptide, or any fragment thereof.

In an embodiment of the present invention, a gene delivery vector containing the polynucleotide, or functional fragment thereof is provided. Preferably, the gene delivery vector contains the polynucleotide, or functional fragment thereof comprising an isolated and purified polynucleotide encoding a human BMSOTR having the sequence as set forth in SEQ ID NO:1.

It will also be appreciated by those skilled in the pertinent art that in addition to the primers disclosed herein (e.g., SEQ ID NO:5-10), a longer oligonucleotide probe, or mixtures of probes, for example, degenerate probes, can be used to detect longer, or more complex, nucleic acid sequences, such as, for example, genomic or full length DNA. In such cases, the probe may comprise at least 20-300 nucleotides, preferably, at least 30-100 nucleotides, and more preferably, 50-100 nucleotides.

The present invention also provides methods of obtaining the full length sequence of the BMSOTR polypeptide as described herein. In one instance, the method of multiplex cloning was devised as a means of extending large numbers of bioinformatic gene predictions into full length sequences by multiplexing probes and cDNA libraries in an effort to minimize the overall effort typically required for cDNA cloning. The method relies on the conversion of plasmid-based, directionally cloned cDNA libraries into a population of pure, covalently-closed, circular, single-stranded molecules and long biotinylated DNA oligonucleotide probes designed from predicted gene sequences.

For such a multiplex cloning method, (see, for example, Example 3 herein), probes and libraries are subjected to solution hybridization in a formamide buffer which has been found to be superior to aqueous buffers typically used in other biotin/streptavidin cDNA capture methods (e.g., GeneTrapper). The hybridization is performed without prior knowledge of the clones represented in the libraries. Hybridization is performed two times. After the first selection, the isolated sequences are screened with PCR primers specific for the targeted clones. The second hybridization is carried out with only those oligo probes whose gene-specific PCR assays give positive results.

The secondary hybridization serves to ‘normalize’ the selected library, thereby decreasing the amount of screening needed to identify particular clones. The method is robust and sensitive. Typically, dozens of cDNAs are isolated for any one particular gene, thereby increasing the chances of obtaining a full length cDNA. The entire complexity of any cDNA library is screened in the solution hybridization process, which is advantageous for finding rare sequences. The procedure is scalable, with 50 oligonucleotide probes per experiment currently being used, although this is not to be considered a limiting number.

Using bioinformatic predicted gene sequence, the following types of PCR primers and cloning oligos can be designed: A) PCR primer pairs that reside within a single predicted exon; B) PCR primer pairs that cross putative exon/intron boundaries; and C) 80mer antisense and sense oligos containing a biotin moiety on the 5′ end. The primer pairs of the A type above are optimized on human genomic DNA; the B type primer pairs are optimized on a mixture of first strand cDNAs made with and without reverse transcriptase. Primers are optimized using mRNA derived from appropriate tissues sources, for example, brain, lung, uterus, cartilage, and testis poly A+ RNA.

The information obtained with the B type primers is used to assess those putative expressed sequences which can be experimentally observed to have reverse transcriptase-dependent expression. The primer pairs of the A type are less stringent in terms of identifying expressed sequences. However, because they amplify genomic DNA as well as cDNA, their ability to amplify genomic DNA provides for the necessary positive control for the primer pair. Negative results with the B type are subject to the caveat that the sequence(s) may not be expressed in the tissue first strand that is under examination.

The biotinylated 80-mer oligonucleotides are added en mass to pools of single strand cDNA libraries. Up to 50 probes have been successfully used on pools for 15 different libraries. After the primary selection is performed, all of the captured DNA is repaired to double strand form using the T7 primer for the commercial libraries in pCMVSPORT, and the Sp6 primer for other constructed libraries in pSPORT. The resulting DNA is electroporated into E. coli DH12S and plated onto 150 mm plates with nylon filters. The cells are scraped and a frozen stock is made, thereby comprising the primary selected library.

One-fifth of the library is generally converted into single strand form and the DNA is assayed with gene specific primer pairs (GSPs). The next round of solution hybridization capture is carried out with 80 mer oligos for only those sequences that are positive with the gene-specific-primers. After the second round, the captured single strand DNAs are repaired with a pool of GSPs, where only the primer complementary to polarity of the single-stranded circular DNA is used (i.e., the antisense primer for pCMVSPORT and pSPORT1 and the sense primer for pSPORT2).

The resulting colonies are screened by PCR using the GSPs. Typically, greater than 80% of the clones are positive for any given GSP. The entire 96 well block of clones is subjected to “mini-prep”, as known in the art, and each of clones is sized by either PCR or restriction enzyme digestion. A selection of different sized clones for each targeted sequence is chosen for transposon-hopping and DNA sequencing.

Preferably, as for established cDNA cloning methods used by the skilled practitioner, the libraries employed are of high quality. High complexity and large average insert size are optimal. High Pressure Liquid Chromatography (HPLC) may be employed as a means of fractionating cDNA for the purpose of constructing libraries.

Another embodiment of the present invention provides a method of identifying full-length genes encoding the disclosed polypeptide. The BMSOTR polynucleotide of the present invention, the polynucleotide encoding the BMSOTR polypeptide of the present invention, or the polypeptide encoded by the deposited clone(s) preferably represent the complete coding region (i.e., full-length gene).

Several methods are known in the art for the identification of the 5′ or 3′ noncoding and/or coding portions of a given gene. The methods described herein are exemplary and should not be construed as limiting the scope of the invention. These methods include, but are not limited to, filter probing, clone enrichment using specific probes, and protocols similar or identical to 5′ and 3′ “RACE” protocols that are well known in the art. For instance, a method similar to 5′ RACE is available for generating the missing 5′ end of a desired full-length transcript. (Fromont-Racine et al., Nucleic Acids Res. 21(7):1683-1684 (1993)).

Briefly, in the RACE method, a specific RNA oligonucleotide is ligated to the 5′ ends of a population of RNA presumably containing full-length gene RNA transcripts. A primer set containing a primer specific to the ligated RNA oligonucleotide and a primer specific to a known sequence of the gene of interest is used to PCR amplify the 5′ portion of the desired full-length gene. This amplified product may then be sequenced and used to generate the full-length gene.

The above method utilizes total RNA isolated from the desired source, although poly-A+ RNA can be used. The RNA preparation is treated with phosphatase, if necessary, to eliminate 5′ phosphate groups on degraded or damaged RNA that may interfere with the later RNA ligase step. The phosphatase is preferably inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase.

The above-described modified RNA preparation is used as a template for first strand cDNA synthesis employing a gene specific oligonucleotide. The first strand synthesis reaction is used as a template for PCR amplification of the desired 5′ end using a primer specific to the ligated RNA oligonucleotide and a primer specific to the known sequence of the gene of interest. The resultant product is then sequenced and analyzed to confirm that the 5′ end sequence belongs to the desired gene. It may also be advantageous to optimize the RACE protocol to increase the probability of isolating additional 5′ or 3′ coding or non-coding sequences. Various methods of optimizing a RACE protocol are known in the art; for example, a detailed description summarizing these methods can be found in B. C. Schaefer, Anal. Biochem., 227:255-273, (1995).

An alternative method for carrying out 5′ or 3′ RACE for the identification of coding or non-coding nucleic acid sequences is provided by Frohman, M. A., et al., Proc. Nat'l. Acad. Sci. USA, 85:8998-9002 (1988). Briefly, a cDNA clone missing either the 5′ or 3′ end can be reconstructed to include the absent base pairs extending to the translational start or stop codon, respectively. In some cases, cDNAs are missing the start of translation for an encoded product. A brief description of a modification of the original 5′ RACE procedure is as follows. Poly A+ or total RNA is reverse transcribed with Superscript II (Gibco/BRL) and an antisense or an I complementary primer specific to any one of the cDNA sequences provided as SEQ ID NOS:1 and 3. The primer is removed from the reaction with a Microcon Concentrator (Amicon). The first-strand cDNA is then tailed with dATP and terminal deoxynucleotide transferase (Gibco/BRL). Thus, an anchor sequence is produced which is needed for PCR amplification. The second strand is synthesized from the dA-tail in PCR buffer, Taq DNA polymerase (Perkin-Elmer Cetus), an oligo-dT primer containing three adjacent restriction sites (XhoIJ Sail and ClaI) at the 5′ end and a primer containing just these restriction sites. This double-stranded cDNA is PCR amplified for 40 cycles with the same primers, as well as a nested cDNA-specific antisense primer. The PCR products are size-separated on an ethidium bromide-agarose gel and the region of gel containing cDNA products having the predicted size of missing protein-coding DNA is removed.

cDNA is purified from the agarose with the Magic PCR Prep kit (Promega), restriction digested with XhoI or SalI, and ligated to a plasmid such as pBluescript SKII (Stratagene) at XhoI and EcoRV sites. This DNA is transformed into bacteria and the plasmid clones sequenced to identify the correct protein-coding inserts. Correct 5′ ends are confirmed by comparing this sequence with the putatively identified homologue and overlap with the partial cDNA clone. Similar methods known in the art and/or commercial kits are used to amplify and recover 3′ ends.

Several quality-controlled kits are commercially available for purchase. Similar reagents and methods to those above are supplied in kit form from Gibco/BRL for both 5′ and 3′ RACE for recovery of full length genes. A second kit is available from Clontech which is a modification of a related technique, called single-stranded ligation to single-stranded cDNA, (SLIC), developed by Dumas et al., Nucleic Acids Res., 19:5227-32(1991). The major difference in the latter procedure is that the RNA is alkaline hydrolyzed after reverse transcription and RNA ligase is used to join a restriction site-containing anchor primer to the first-strand cDNA. This obviates the necessity for the dA-tailing reaction which results in a polyT stretch that can impede sequencing.

An alternative to generating 5′ or 3′ cDNA from RNA is to use cDNA library double-stranded DNA. An asymmetric PCR-amplified antisense cDNA strand is synthesized with an antisense cDNA-specific primer and a plasmid-anchored primer. These primers are removed and a symmetric PCR reaction is performed with a nested cDNA-specific antisense primer and the plasmid-anchored primer.

Also encompassed by the present invention are polynucleotide sequences that are capable of hybridizing to the novel BMSOTR nucleic acid sequence as set forth in SEQ ID NO:1 under various conditions of stringency. Hybridization conditions are typically based on the melting temperature (Tₘ) of the nucleic acid binding complex or probe (see, G. M. Wahl and S. L. Berger, 1987; Methods Enzymol., 152:399-407 and A. R. Kimmel, 1987; Methods of Enzymol., 152:507-511), and may be used at a defined stringency. For example, included in the present invention are sequences capable of hybridizing under moderately stringent conditions to the BMSOTR sequence of SEQ ID NO:1 and other sequences which are degenerate to those which encode the novel BMSOTR polypeptide. For example, a non-limiting example of moderate stringency conditions include prewashing solution of 2×SSC, 0.5% SDS, 1.0 mM EDTA, pH 8.0, and hybridization conditions of 50° C., 5×SSC, overnight.

The nucleic acid sequence encoding the BMSOTR protein of the present invention may be extended by utilizing a partial nucleotide sequence and employing various methods known in the art to detect upstream sequences such as promoters and regulatory elements. For example, one method that can be employed is restriction-site PCR, which utilizes universal primers to retrieve unknown sequence adjacent to a known locus (See, e.g., G. Sarkar, 1993, PCR Methods Applic., 2:318-322). In particular, genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR may also be used to amplify or extend sequences using divergent primers based on a known region or sequence (T. Triglia et al., 1988, Nucleic Acids Res., 16:8186). The primers may be designed using OLIGO 4.06 Primer Analysis software (National Biosciences, Inc., Plymouth, Minn.), or another appropriate program, to be 22-30 nucleotides in length, to have a GC content of 50% or more, and to anneal to the target sequence at temperatures about 68° C.-72° C. The method uses several restriction enzymes to generate a suitable fragment in the known region of a gene. The fragment is then circularized by intramolecular ligation and used as a PCR template.

Another method which may be used to amplify or extend sequences is capture PCR which involves PCR amplification of DNA fragments adjacent to a known sequence in human and yeast artificial chromosome (YAC) DNA (M. Lagerstrom et al., 1991, PCR Methods Applic., 1:111-119). In this method, multiple restriction enzyme digestions and ligations may also be used to place an engineered double-stranded sequence into an unknown portion of the DNA molecule before performing PCR. J. D. Parker et al. (1991; Nucleic Acids Res., 19:3055-3060) provide another method which may be used to retrieve unknown sequences. Bacterial artificial chromosomes (BACs) are also used for such applications. In addition, PCR, nested primers, and PROMOTERFINDER libraries can be used to “walk” genomic DNA (Clontech, Palo Alto, Calif.). This process avoids the need to screen libraries and is useful in finding intron/exon junctions.

When screening for full-length cDNAs, it is preferable to use libraries that have been size-selected to include larger cDNAs. Also, random-primed libraries are also preferable, since such libraries will contain more sequences that comprise the 5′ regions of genes. The use of a randomly primed library may be especially preferable for situations in which an oligo d(T) library does not yield a full-length cDNA. Genomic libraries may be useful for extension of sequence into the 5′ and 3′ non-transcribed regulatory regions.

The embodiments of the present invention can be practiced using methods for DNA sequencing which are well known and generally available in the art. The methods may employ such enzymes as the Klenow fragment of DNA polymerase I, SEQUENASE (US Biochemical Corp. Cleveland, Ohio), Taq polymerase (PE Biosystems), thermostable T7 polymerase (Amersham Pharmacia Biotech, Piscataway, N.J.), or combinations of recombinant polymerases and proofreading exonucleases such as the ELONGASE Amplification System marketed by Life Technologies (Gaithersburg, Md.). Preferably, the process is automated with machines such as the Hamilton Micro Lab 2200 (Hamilton, Reno, Nev.), Peltier Thermal Cycler (PTC200; MJ Research, Watertown, Mass.) and the ABI Catalyst and 373 and 377 DNA sequencers (PE Biosystems). Commercially available capillary electrophoresis systems may be used to analyze the size or confirm the nucleotide sequence of sequencing or PCR products. Capillary electrophoresis is especially preferable for the sequencing of small pieces of DNA, which might be present in limited amounts in a particular sample.

In another embodiment of the present invention, polynucleotide sequences or portions thereof which encode a BMSOTR polypeptide or peptides can comprise recombinant DNA molecules to direct the expression of BMSOTR polypeptide products, peptide fragments, or functional equivalents thereof, in appropriate host cells. Because of the inherent degeneracy of the genetic code, other DNA sequences, which encode substantially the same or a functionally equivalent amino acid sequence, may be produced and these sequences may be used to clone and express the BMSOTR proteins as described.

As will be appreciated by those having skill in the art, it may be advantageous to produce BMSOTR polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

The nucleotide sequences of the present invention can be engineered using methods generally known in the art in order to alter the BMSOTR polypeptide-encoding sequences for a variety of reasons, including, but not limited to, alterations which modify the cloning, processing, and/or expression of the gene product. DNA shuffling by random fragmentation, PCR reassembly of gene fragments and synthetic oligonucleotides may be used to engineer the nucleotide sequences. For example, site-directed mutagenesis may be used to insert new restriction sites, alter glycosylation patterns, change codon preference, produce splice variants, or introduce mutations, and the like.

In another embodiment of the present invention, natural, modified, or recombinant nucleic acid sequences encoding the BMSOTR polypeptide may be ligated to a heterologous sequence to encode a fusion (or chimeric or hybrid) protein. For example, a fusion protein can comprise all or part of the amino acid sequence as set forth in SEQ ID NO:2 and an amino acid sequence of an Fc portion (or constant region) of a human immunoglobulin protein. The fusion protein may further comprise an amino acid sequence that differs from SEQ ID NO:2 only by conservative substitutions. As another example, to screen peptide libraries for inhibitors of BMSOTR activity, it may be useful to generate a chimeric BMSOTR protein that can be recognized by a commercially available antibody. A fusion protein may also be engineered to contain a cleavage site located between the BMSOTR protein-encoding sequence and the heterologous protein sequence, so that the BMSOTR protein may be cleaved and purified away from the heterologous moiety.

In a further embodiment, sequences encoding the BMSOTR polypeptide may be synthesized in whole, or in part, using chemical methods well known in the art (see, for example, M. H. Caruthers et al., 1980, Nucl. Acids Res. Symp. Ser., 215-223 and T. Horn et al., 1980, Nucl. Acids Res. Symp. Ser., 225-232). Alternatively, the BMSOTR protein itself, or a fragment or portion thereof, may be produced using chemical methods to synthesize the amino acid sequence of the BMSOTR polypeptide, or a fragment or portion thereof. For example, peptide synthesis can be performed using various solid-phase techniques (J. Y. Roberge et al., 1995, Science, 269:202-204) and automated synthesis can be achieved, for example, using the ABI 431A Peptide Synthesizer (PE Biosystems).

The newly synthesized BMSOTR polypeptide or peptide can be substantially purified by preparative high performance liquid chromatography (e.g., T. Creighton, 1983, Proteins, Structures and Molecular Principles, W. H. Freeman and Co., New York, N.Y.), by reverse-phase high performance liquid chromatography (HPLC), or other purification methods as known and practiced in the art. The composition of the synthetic peptides may be confirmed by amino acid analysis or sequencing (e.g., the Edman degradation procedure; Creighton, supra). In addition, the amino acid sequence of a BMSOTR polypeptide, or any portion thereof, can be altered during direct synthesis and/or combined using chemical methods with sequences from other proteins, or any part thereof, to produce a variant polypeptide.

To express a biologically active BMSOTR polypeptide or peptide, the nucleotide sequences encoding the BMSOTR polypeptide, or functional equivalents, may be inserted into an appropriate expression vector, i.e., a vector, which contains the necessary elements for the transcription and translation of the inserted coding sequence.

In an embodiment of the present invention, an expression vector contains an isolated and purified polynucleotide sequence as set forth in SEQ ID NO:1 encoding human BMSOTR, or a functional fragment thereof, in which the human BMSOTR comprises the amino acid sequence as set forth in SEQ ID NO:2. Alternatively, an expression vector can contain the complement of the aforementioned BMSOTR nucleic acid sequence.

Expression vectors derived from retroviruses, adenovirus, herpes or vaccinia viruses, or from various bacterial plasmids can be used for the delivery of nucleotide sequences to a target organ, tissue or cell population. Methods, which are well known to those skilled in the art, may be used to construct expression vectors containing sequences encoding the BMSOTR polypeptide along with appropriate transcriptional and translational control elements. These methods include in vitro recombinant DNA techniques, synthetic techniques, and in vivo genetic recombination. Such techniques are described in the most recent edition of J. Sambrook et al., 1989, Molecular Cloning, A Laboratory Manual, Cold Spring Harbor Press, Plainview, N.Y. and in F. M. Ausubel et al., 1989, Current Protocols in Molecular Biology, John Wiley & Sons, New York, N.Y.

A variety of expression vector/host systems may be utilized to contain and express sequences encoding the BMSOTR polypeptide or peptides. Such expression vector/host systems include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus (CaMV) and tobacco mosaic virus (TMV)), or with bacterial expression vectors (e.g., Ti or pBR322 plasmids); or animal cell systems, including mammalian cell systems. The host cell employed is not limiting to the present invention. Preferably, the host cell of the invention contains an expression vector comprising an isolated and purified polynucleotide having a nucleic acid sequence selected from SEQ ID NO:1 and encoding the BMSOTR of this invention, or a functional fragment thereof, comprising an amino acid sequence as set forth in SEQ ID NO:2.

Bacterial artificial chromosomes (BACs) may be used to deliver larger fragments of DNA than can be contained and expressed in a plasmid vector. BACs are vectors used to clone DNA sequences of 100-300 kb, on average 150 kb, in size in E. coli cells. BACs are constructed and delivered via conventional delivery methods (e.g., liposomes, polycationic amino polymers, or vesicles) for therapeutic purposes.

“Control elements” or “regulatory sequences” are those non-translated regions of the vector, e.g., enhancers, promoters, 5′ and 3′ untranslated regions, which interact with host cellular proteins to carry out transcription and translation. Such elements may vary in their strength and specificity. Depending on the vector system and host utilized, any number of suitable transcription and translation elements, including constitutive and inducible promoters, may be used. Specific initiation signals may also be used to achieve more efficient translation of sequences encoding a BMSOTR polypeptide. Such signals include the ATG initiation codon and adjacent sequences. In cases where sequences encoding a BMSOTR polypeptide, its initiation codon, and upstream sequences are inserted into the appropriate expression vector, no additional transcriptional or translational control signals may be needed. However, in cases where only a BMSOTR coding sequence, or a fragment thereof, is inserted, exogenous translational control signals, including the ATG initiation codon, are optimally provided. Furthermore, the initiation codon should be in the correct reading frame to insure translation of the entire insert. Exogenous translational elements and initiation codons can be of various origins, both natural and synthetic. The efficiency of expression can be enhanced by the inclusion of enhancers which are appropriate for the particular cell system that is used, such as those described in the literature (see, e.g., D. Scharf et al., 1994, Results Probl. Cell Differ., 20:125-162).

In bacterial systems, a number of expression vectors may be selected, depending upon the use intended for the expressed BMSOTR product. For example, when large quantities of expressed protein are needed for the generation of antibodies, vectors that direct high level expression of fusion proteins that can be readily purified may be used. Such vectors include, but are not limited to, the multifunctional E. coli cloning and expression vectors such as BLUESCRIPT (Stratagene), in which the sequence encoding the BMSOTR polypeptide can be ligated into the vector in-frame with sequences for the amino-terminal Met and the subsequent 7 residues of β-galactosidase, so that a hybrid protein is produced; pIN vectors (see, G. Van Heeke and S. M. Schuster, 1989, J. Biol. Chem . . . , 264:5503-5509); and the like. pGEX vectors (Promega, Madison, Wis.) can also be used to express foreign polypeptides as fusion proteins with glutathione S-transferase (GST). In general, such fusion proteins are soluble and can be easily purified from lysed cells by adsorption to glutathione-agarose beads followed by elution in the presence of free glutathione. Proteins made in such systems can be designed to include heparin, thrombin, or factor XA protease cleavage sites so that the cloned polypeptide of interest can be released from the GST moiety at will.

In mammalian host cells, a number of viral-based expression systems can be utilized. In cases where an adenovirus is used as an expression vector, sequences encoding the BMSOTR polypeptide may be ligated into an adenovirus transcription/translation complex containing the late promoter and tripartite leader sequence. Insertion into a non-essential E1 or E3 region of the viral genome may be used to obtain a viable virus which is capable of expressing a BMSOTR polypeptide in infected host cells (J. Logan and T. Shenk, 1984, Proc. Natl. Acad. Sci., 81:3655-3659). In addition, transcription enhancers, such as the Rous sarcoma virus (RSV) enhancer, may be used to increase expression in mammalian host cells. Other expression systems can also be used, such as, but not limited to yeast, plant, and insect vectors.

Moreover, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells having specific cellular machinery and characteristic mechanisms for such post-translational activities (e.g., CHO, HeLa, MDCK, HEK293, and WI 38) are available from the American Type Culture Collection (ATCC), 10801 University Boulevard, Manassas, Va. 20110-2209, and may be chosen to ensure the correct modification and processing of the heterologous protein.

Host cells transformed with vectors containing nucleotide sequences encoding a BMSOTR protein, or fragments thereof, may be cultured under conditions suitable for the expression and recovery of the protein from cell culture. The protein produced by a recombinant cell may be secreted or contained intracellularly depending on the sequence and/or the vector used. As will be understood by those having skill in the art, expression vectors containing polynucleotides which encode a BMSOTR protein can be designed to contain signal sequences which direct secretion of the BMSOTR protein through a prokaryotic or eukaryotic cell membrane. Other constructions can be used to join nucleic acid sequences encoding a BMSOTR protein to a nucleotide sequence encoding a polypeptide domain, which will facilitate purification of soluble proteins. Such purification facilitating domains include, but are not limited to, metal chelating peptides such as histidine-tryptophan modules that allow purification on immobilized metals; protein A domains that allow purification on immobilized immunoglobulin; and the domain utilized in the FLAGS extension/affinity purification system (Immunex Corp., Seattle, Wash.). The inclusion of cleavable linker sequences such as those specific for Factor XA or enterokinase (Invitrogen, San Diego, Calif.) between the purification domain and the BMSOTR protein may be used to facilitate purification. One such expression vector provides for expression of a fusion protein containing BMSOTR and a nucleic acid encoding 6 histidine residues preceding a thioredoxin or an enterokinase cleavage site. The histidine residues facilitate purification on IMAC (immobilized metal ion affinity chromatography) as described by J. Porath et al., 1992, Prot. Exp. Purif., 3:263-281, while the enterokinase cleavage site provides a means for purifying the 6 histidine residue tag from the fusion protein. For a discussion of suitable vectors for fusion protein production, see D. J. Kroll et al., 1993; DNA Cell Biol., 12:441-453.

Any number of selection systems may be used to recover transformed cell lines. These include, but are not limited to, the Herpes Simplex Virus thymidine kinase (HSV TK), (M. Wigler et al., 1977, Cell, 11:223-32) and adenine phosphoribosyltransferase (I. Lowy et al., 1980, Cell, 22:817-23) genes which can be employed in tk⁻ or aprt⁻ cells, respectively. Also, anti-metabolite, antibiotic or herbicide resistance can be used as the basis for selection; for example, dhfr, which confers resistance to methotrexate (M. Wigler et al., 1980, Proc. Natl. Acad. Sci., 77:3567-70); npt, which confers resistance to the aminoglycosides neomycin and G418 (F. Colbere-Garapin et al., 1981, J. Mol. Biol., 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (S. C. Hartman and R. C. Mulligan, 1988, Proc. Natl. Acad. Sci., 85:8047-51). Recently, the use of visible markers has gained popularity with such markers as the anthocyanins, β-glucuronidase and its substrate GUS, and luciferase and its substrate luciferin, which are widely used not only to identify transformants, but also to quantify the amount of transient or stable protein expression that is attributable to a specific vector system (C. A. Rhodes et al., 1995, Methods Mol. Biol., 55:121-131).

Although the presence or absence of marker gene expression suggests that the gene of interest is also present, the presence and expression of the desired gene of interest may need to be confirmed. For example, if the nucleic acid sequence encoding the BMSOTR polypeptide is inserted within a marker gene sequence, recombinant cells containing a polynucleotide sequence encoding the BMSOTR polypeptide can be identified by the absence of marker gene function. Alternatively, a marker gene can be placed in tandem with a sequence encoding the BMSOTR polypeptide under the control of a single promoter. Expression of the marker gene in response to induction or selection typically indicates co-expression of the tandem gene.

A wide variety of labels and conjugation techniques are known and employed by those skilled in the art and may be used in various nucleic acid and amino acid assays. Means for producing labeled hybridization or PCR probes for detecting sequences related to polynucleotides encoding a BMSOTR polypeptide include oligo-labeling, nick translation, end-labeling, or PCR amplification using a labeled nucleotide. Alternatively, the sequences encoding a BMSOTR polypeptide of this invention, or any portion or fragment thereof, can be cloned into a vector for the production of an mRNA probe. Such vectors are known in the art, are commercially available, and may be used to synthesize RNA probes in vitro by addition of an appropriate RNA polymerase, such as T7, T3, or SP(6) and labeled nucleotides. These procedures may be conducted using a variety of commercially available kits (e.g., Amersham Pharmacia Biotech, Promega and U.S. Biochemical Corp.). Suitable reporter molecules or labels which can be used include radionucleotides, enzymes, fluorescent, chemiluminescent, or chromogenic agents, as well as substrates, cofactors, inhibitors, magnetic particles, and the like.

Alternatively, host cells which contain the nucleic acid sequence coding for a BMSOTR polypeptide of the invention and which express the BMSOTR polypeptide product may be identified by a variety of procedures known to those having skill in the art. These procedures include, but are not limited to, DNA-DNA or DNA-RNA hybridizations and protein bioassay or immunoassay techniques, including membrane, solution, or chip based technologies, for the detection and/or quantification of nucleic acid or protein.

The presence of polynucleotide sequences encoding BMSOTR polypeptides can be detected by DNA-DNA or DNA-RNA hybridization, or by amplification using probes, portions, or fragments of polynucleotides encoding a BMSOTR polypeptide. Nucleic acid amplification based assays involve the use of oligonucleotides or oligomers based on the nucleic acid sequences encoding a BMSOTR polypeptide to detect transformants containing DNA or RNA encoding a BMSOTR polypeptide.

In addition to recombinant production, fragments of the BMSOTR polypeptide may be produced by direct peptide synthesis using solid phase techniques (J. Merrifield, 1963, J. Am. Chem. Soc., 85:2149-2154). Protein synthesis may be performed using manual techniques or by automation. Automated synthesis may be achieved, for example, using ABI 431A Peptide Synthesizer (PE Biosystems). Various fragments of the BMSOTR polypeptide can be chemically synthesized separately and then combined using chemical methods to produce the full length molecule.

### Diagnostic Assays

In another embodiment of the present invention, antibodies which specifically bind to the BMSOTR polypeptide may be used for the diagnosis of conditions or diseases characterized by expression (or overexpression) of the BMSOTR polynucleotide or polypeptide, or in assays to monitor patients being treated with the BMSOTR polypeptide, or agonists, antagonists, or inhibitors of the novel BMSOTR. The antibodies useful for diagnostic purposes can be prepared in the same manner as those described herein for use in therapeutic methods. Diagnostic assays for the BMSOTR polypeptide include methods which utilize the antibody and a label to detect the protein in human body fluids or extracts of cells or tissues. The antibodies may be used with or without modification, and may be labeled by joining them, either covalently or non-covalently, with a reporter molecule. A wide variety of reporter molecules known to those in the art may be used, several of which are described herein.

Another embodiment of the present invention contemplates a method of detecting a BMSOTR homologue, or an antibody-reactive fragment thereof, in a sample. The method comprises a) contacting the sample with an antibody specific for a BMSOTR polypeptide of the present invention, or an antigenic fragment thereof, under conditions in which an antigen-antibody complex can form between the antibody and the polypeptide or antigenic fragment thereof in the sample; and b) detecting the antigen-antibody complex formed in step a), wherein detection of the complex indicates the presence of the BMSOTR polypeptide, or an antigenic fragment thereof, in the sample.

Several assay protocols including enzyme-linked immunosorbent assay (ELISA), radioimmunoassay (RIA), and fluorescence activated cell sorting (FACS) for measuring a BMSOTR polypeptide are known in the art and provide a basis for diagnosing altered or abnormal levels of BMSOTR polypeptide expression. Normal or standard values for BMSOTR polypeptide expression are established by combining body fluids or cell extracts taken from normal mammalian subjects, preferably human, with antibody to the BMSOTR polypeptide under conditions suitable for complex formation. The amount of standard complex formation may be quantified by various methods; photometric means are preferred. Quantities of BMSOTR polypeptide expressed in a subject or test sample, control sample, and disease samples from biopsied tissues are compared with the standard values. Deviation between standard and subject values establishes the parameters for diagnosing disease.

A variety of protocols for detecting and measuring the expression of BMSOTR polypeptide using either polyclonal or monoclonal antibodies specific for the polypeptide, or epitopic portions thereof, are known and practiced in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive with two non-interfering epitopes on a BMSOTR polypeptide is preferred, but a competitive binding assay may also be employed. These and other assays are described in the art as represented by the publication of R. Hampton et al., 1990; Serological Methods, a Laboratory Manual, APS Press, St Paul, Minn. and D. E. Maddox et al., 1983; J. Exp. Med., 158:1211-1216).

Another embodiment of the present invention encompasses a method of using a BMSOTR-encoding polynucleotide sequence to isolate and/or purify a molecule or compound in a sample, wherein the molecule or compound specifically binds to the polynucleotide. The method comprises: a) combining a BMSOTR-encoding polynucleotide of the invention with a sample undergoing testing to determine if the sample contains the molecule or compound, under conditions to allow specific binding; b) detecting specific binding between the BMSOTR-encoding polynucleotide and the molecule or compound, if present; c) recovering the bound polynucleotide; and d) separating the polynucleotide from the molecule or compound, thereby obtaining a purified or substantially purified molecule or compound.

This invention also relates to a method of using BMSOTR polynucleotides as diagnostic reagents. For example, the detection of a mutated form of the BMSOTR gene associated with a dysfunction can provide a diagnostic tool that can add to or define diagnosis of a disease or susceptibility to a disease which results from under-expression, over-expression, or altered expression of BMSOTR. Individuals carrying mutations in the BMSOTR gene may be detected at the DNA level by a variety of techniques.

Nucleic acids for diagnosis may be obtained from various sources of a subject, for example, from cells, tissue, blood, urine, saliva, tissue biopsy or autopsy material. Genomic DNA may be used directly for detection or may be amplified by using PCR or other amplification techniques prior to analysis. RNA or cDNA may also be used in similar fashion. Deletions and insertions in a BMSOTR-encoding polynucleotide can be detected by a change in size of the amplified product compared with that of the normal genotype. Hybridizing amplified DNA to labeled GPCR polynucleotide sequences can identify point mutations. Perfectly matched sequences can be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing. See, for example, Myers et al., Science (1985) 230:1242. Sequence changes at specific locations may also be revealed by nuclease protection assays, such as RNase and S1 protection or the chemical cleavage method. (See Cotton et al., Proc. Natl. Acad. Sci., USA (1985) 85:43297-4401).

In another embodiment, an array of oligonucleotide probes comprising the BMSOTR nucleotide sequence or fragments thereof can be constructed to conduct efficient screening of, for example, genetic mutations. Array technology methods are well known, have general applicability and can be used to address a variety of questions in molecular genetics, including gene expression, genetic linkage, and genetic variability (see for example: M. Chee et al., Science, 274:610-613, 1996).

Yet another aspect of the present invention involves a method of screening a library of molecules or compounds with a BMSOTR-encoding polynucleotide to identify at least one molecule or compound therein which specifically binds to the BMSOTR polynucleotide sequence. Such a method includes a) combining a BMSOTR-encoding polynucleotide of the present invention with a library of molecules or compounds under conditions to allow specific binding; and b) detecting specific binding, thereby identifying a molecule or compound, which specifically binds to a BMSOTR-encoding polynucleotide sequence, wherein the library is selected from DNA molecules, RNA molecules, artificial chromosome constructions, PNAs, peptides and proteins.

The present invention provides diagnostic assays for determining or monitoring through detection of a mutation in the BMSOTR gene (polynucleotide) described herein susceptibility to the following conditions, diseases, or disorders: cancers; anorexia; Prader-Willi syndrome, bulimia; asthma; Alzheimer's disease, Parkinson's disease; acute heart failure; hypotension; hypertension; urinary retention; osteoporosis; angina pectoris; myocardial infarction; ulcers; asthma; allergies; benign prostatic hypertrophy; reproductive disorders; memory and learning disorders; behavioral disorders; and psychotic and neurological disorders, including anxiety, depression, headache, migraine, schizophrenia, obsessive-compulsive disorder, manic depression, delirium, dementia, severe mental retardation and dyskinesias, such as Huntington's disease or Gilles de la Tourette's syndrome.

In addition, such diseases, disorder, or conditions, can be diagnosed by methods of determining from a sample derived from a subject having an abnormally decreased or increased level of BMSOTR polypeptide or BMSOTR mRNA. Decreased or increased expression can be measured at the RNA level using any of the methods well known in the art for the quantification of polynucleotides, such as, for example, PCR, RT-PCR, RNase protection, Northern blotting and other hybridization methods. Assay techniques that can be used to determine levels of a protein, such as BMSOTR in a sample derived from a host are well known to those of skill in the art. Such assay methods include, without limitation, radioimmunoassays, competitive-binding assays, Western Blot analysis and ELISA assays.

In another of its aspects, this invention relates to a kit for detecting and diagnosing a BMSOTR-associated disease or susceptibility to such a disease, which comprises a BMSOTR or BMSOTR variant polynucleotide, preferably the nucleotide sequence of SEQ ID NOS:1 or 3, or a fragment thereof; or a nucleotide sequence complementary to the BMSOTR polynucleotide of SEQ ID NOS:1 or 3; or a BMSOTR or BMSOTR variant polypeptide, preferably the polypeptide of SEQ ID NOS:2 or 4, or a fragment thereof; or an antibody to the BMSOTR or BMSOTR variant polypeptide, preferably to the polypeptide of SEQ ID NOS:2 or 4, an epitope-containing portion thereof, or combinations of the foregoing. It will be appreciated that in any such kit, any of the previously mentioned components may comprise a substantial component. Also preferably included are instructions for use.

The BMSOTR polynucleotides which may be used in the diagnostic assays according to the present invention include oligonucleotide sequences, complementary RNA and DNA molecules, and PNAs. The polynucleotides may be used to detect and quantify BMSOTR-encoding nucleic acid expression in biopsied tissues in which expression (or under- or over-expression) of the BMSOTR polynucleotide may be determined, as well as correlated with disease. The diagnostic assays may be used to distinguish between the absence of BMSOTR, the presence of BMSOTR, or the excess expression of BMSOTR, and to monitor the regulation of BMSOTR polynucleotide levels during therapeutic treatment or intervention.

In a related aspect, hybridization with PCR probes which are capable of detecting polynucleotide sequences, including genomic sequences, encoding a BMSOTR polypeptide according to the present invention, or closely related molecules, may be used to identify nucleic acid sequences which encode a BMSOTR polypeptide. The specificity of the probe, whether it is made from a highly specific region, for example, about 8 to 10 contiguous nucleotides in the 5′ regulatory region, or a less specific region, for example, especially in the 3′ coding region, and the stringency of the hybridization or amplification (maximal, high, intermediate, or low) will determine whether the probe identifies only naturally occurring sequences encoding BMSOTR polypeptide, alleles thereof, or related sequences, as understood by the skilled practitioner.

Probes may also be used for the detection of related sequences, and should preferably contain at least 50% of the nucleotides encoding the BMSOTR polypeptide. The hybridization probes or primers of this invention may be DNA or RNA and may be derived from the nucleotide sequences of SEQ ID NO:1, or may be derived from genomic sequence, including promoter, enhancer elements, and introns of the naturally occurring BMSOTR protein, wherein the probes or primers comprise a polynucleotide sequence capable of hybridizing with a polynucleotide of SEQ ID NO:1, under low, moderate, or high stringency conditions.

Methods for producing specific hybridization probes for DNA encoding the BMSOTR polypeptide include the cloning of a nucleic acid sequence that encodes the BMSOTR polypeptide, or BMSOTR derivatives, into vectors for the production of mRNA probes. Such vectors are known in the art, or are commercially available, and may be used to synthesize RNA probes in vitro by means of the addition of the appropriate RNA polymerases and the appropriate labeled nucleotides. Hybridization probes may be labeled by a variety of detector/reporter groups, including, but not limited to, radionucleotides such as ³²P or ³⁵S, or enzymatic labels, such as alkaline phosphatase coupled to the probe via avidin/biotin coupling systems, and the like.

The polynucleotide sequence encoding the BMSOTR polypeptide of this invention, or fragments thereof, may be used for the diagnosis of disorders associated with expression of BMSOTR. The polynucleotide sequence encoding the BMSOTR polypeptide may be used in Southern or Northern analysis, dot blot, or other membrane-based technologies; in PCR technologies; or in dipstick, pin, ELISA or chip assays utilizing fluids or tissues from patient biopsies to detect the status of, for example, levels of, or overexpression of, BMSOTR, or to detect altered BMSOTR expression or levels. Such qualitative or quantitative methods are commonly practiced in the art.

In a particular aspect, a nucleotide sequence encoding BMSOTR polypeptide as described herein may be useful in assays that detect activation or induction of various neoplasms, cancers, or other BMSOTR-related diseases, disorders, or conditions. The nucleotide sequence encoding a BMSOTR polypeptide may be labeled by standard methods, and added to a fluid or tissue sample from a patient, under conditions suitable for the formation of hybridization complexes. After a suitable incubation period, the sample is washed and the signal is quantified and compared with a standard value. If the amount of signal in the biopsied or extracted sample is significantly altered from that of a comparable control sample, the nucleotide sequence has hybridized with nucleotide sequence present in the sample, and the presence of altered levels of nucleotide sequence encoding the BMSOTR polypeptide in the sample indicates the presence of the associated disease. Such assays may also be used to evaluate the efficacy of a particular therapeutic treatment regimen in animal studies, in clinical trials, or in monitoring the treatment or responsiveness of an individual patient.

Once disease is established and a treatment protocol is initiated, hybridization assays may be repeated on a regular basis to evaluate whether the level of expression in the patient begins to approximate that which is observed in a normal individual. The results obtained from successive assays may be used to show the efficacy of treatment over a period ranging from several days to months.

With respect to tumors or cancer, the presence of an abnormal amount or level of a BMSOTR transcript in biopsied tissue from an individual may indicate a predisposition for the development of the disease, or may provide a means for detecting the disease prior to the appearance of actual clinical symptoms. A more definitive diagnosis of this type may allow health practitioners to employ preventative measures or aggressive treatment earlier, thereby preventing the development or further progression of the tumor or cancer.

Additional diagnostic uses for oligonucleotides designed from the nucleic acid sequences encoding the novel BMSOTR polypeptide of this invention can involve the use of PCR. Such oligomers may be chemically synthesized, generated enzymatically, or produced from a recombinant source. Oligomers will preferably comprise two nucleotide sequences: one with sense orientation (5′→3′) and another with antisense orientation (3′→5′), employed under optimized conditions for identification of a specific gene or condition. The same two oligomers, nested sets of oligomers, or even a degenerate pool of oligomers may be employed under less stringent conditions for detection and/or quantification of closely related DNA or RNA sequences.

Methods suitable for quantifying the expression of BMSOTR include radiolabeling or biotinylating nucleotides, co-amplification of a control nucleic acid, and standard curves onto which the experimental results are interpolated (P. C. Melby et al., 1993, J. Immunol. Methods, 159:235-244; and C. Duplaa et al., 1993, Anal. Biochem., 229-236). The speed of quantifying multiple samples may be accelerated by running the assay in an ELISA format where the oligomer of interest is presented in various dilutions and a spectrophotometric or calorimetric response gives rapid quantification.

In another embodiment of the invention, a compound to be tested can be radioactively, calorimetrically or fluorimetrically labeled using methods well known in the art and incubated with the BMSOTR polypeptide for testing. After incubation, it is determined whether the test compound is bound to the BMSOTR polypeptide. If so, the compound is to be considered a potential agonist or antagonist. Functional assays are performed to determine whether the receptor activity is activated (or enhanced or increased) or inhibited (or decreased or reduced). These assays include, but are not limited to, cell cycle analysis and in vivo tumor formation assays. Responses can also be measured in cells expressing the receptor using signal transduction systems including, but not limited to, protein phosphorylation, adenylate cyclase activity, phosphoinositide hydrolysis, guanylate cyclase activity, ion fluxes (i.e. calcium) and pH changes. These types of responses can either be present in the host cell or introduced into the host cell along with the receptor.

The present invention further embraces a method of screening for candidate compounds capable of modulating the activity of a BMSOTR-encoding polynucleotide. Such a method comprises a) contacting a test compound with a cell, cell membrane or tissue expressing a BMSOTR polypeptide of the invention (e.g., recombinant expression); and b) selecting as candidate modulating compounds those test compounds that modulate activity of the BMSOTR polypeptide. Those candidate compounds which modulate BMSOTR activity are preferably agonists or antagonists, more preferably antagonists of BMSOTR activity.

### Therapeutic Assays

The BMSOTR protein according to this invention may play a role in cell signaling, in cell cycle regulation, and/or in neurological disorders. The BMSOTR protein may further be involved in neoplastic, cardiovascular, and immunological disorders.

Nervous system diseases, disorders, and/or conditions, which can be treated, prevented, and/or diagnosed with the compositions of the invention (e.g., polypeptides, polynucleotides, and/or agonists or antagonists), include, but are not limited to, nervous system injuries, and diseases, disorders, and/or conditions which result in either a disconnection of axons, a diminution or degeneration of neurons, or demyelination. Nervous system lesions which may be treated, prevented, and/or diagnosed in a patient (including human and non-human mammalian patients) according to the invention, include but are not limited to, the following lesions of either the central (including spinal cord, brain) or peripheral nervous systems: (1) ischemic lesions, in which a lack of oxygen in a portion of the nervous system results in neuronal injury or death, including cerebral infarction or ischemia, or spinal cord infarction or ischemia; (2) traumatic lesions, including lesions caused by physical injury or associated with surgery, for example, lesions which sever a portion of the nervous system, or compression injuries; (3) malignant lesions, in which a portion of the nervous system is destroyed or injured by malignant tissue which is either a nervous system associated malignancy or a malignancy derived from non-nervous system tissue; (4) infectious lesions, in which a portion of the nervous system is destroyed or injured as a result of infection, for example, by an abscess or associated with infection by human immunodeficiency virus, herpes zoster, or herpes simplex virus or with Lyme disease, tuberculosis, syphilis; (5) degenerative lesions, in which a portion of the nervous system is destroyed or injured as a result of a degenerative process including but not limited to degeneration associated with Parkinson's disease, Alzheimer's disease, Huntington's chorea, or amyotrophic lateral sclerosis (ALS); (6) lesions associated with nutritional diseases, disorders, and/or conditions, in which a portion of the nervous system is destroyed or injured by a nutritional disorder or disorder of metabolism including but not limited to, vitamin B12 deficiency, folic acid deficiency, Wernicke disease, tobacco-alcohol amblyopia, Marchiafava-Bignami disease (primary degeneration of the corpus callosum), and alcoholic cerebellar degeneration; (7) neurological lesions associated with systemic diseases including, but not limited to, diabetes (diabetic neuropathy, Bell's palsy), systemic lupus erythematosus, carcinoma, or sarcoidosis; (8) lesions caused by toxic substances including alcohol, lead, or particular neurotoxins; and (9) demyelinated lesions in which a portion of the nervous system is destroyed or injured by a demyelinating disease including, but not limited to, multiple sclerosis, human immunodeficiency virus-associated myelopathy, transverse myelopathy or various etiologies, progressive multifocal leukoencephalopathy, and central pontine myelinolysis.

In a preferred embodiment, the polypeptides, polynucleotides, or agonists or antagonists of the invention are used to protect neural cells from the damaging effects of cerebral hypoxia. According to this embodiment, the compositions of the invention are used to treat, prevent, and/or diagnose neural cell injury associated with cerebral hypoxia. In one aspect of this embodiment, the polypeptides, polynucleotides, or agonists or antagonists of the invention are used to treat, prevent, and/or diagnose neural cell injury associated with cerebral ischemia. In another aspect of this embodiment, the polypeptides, polynucleotides, or agonists or antagonists of the invention are used to treat, prevent, and/or diagnose neural cell injury associated with cerebral infarction. In another aspect of this embodiment, the polypeptides, polynucleotides, or agonists or antagonists of the invention are used to treat, prevent, and/or diagnose or prevent neural cell injury associated with a stroke. In a further aspect of this embodiment, the polypeptides, polynucleotides, or agonists or antagonists of the invention are used to treat, prevent, and/or diagnose neural cell injury associated with a heart attack.

The compositions of the invention which are useful for treating or preventing a nervous system disorder may be selected by testing for biological activity in promoting the survival or differentiation of neurons. For example, and not by way of limitation, compositions of the invention which elicit any of the following effects may be useful according to the invention: (1) increased survival time of neurons in culture; (2) increased sprouting of neurons in culture or in vivo; (3) increased production of a neuron-associated molecule in culture or in vivo, e.g., choline acetyltransferase or acetylcholinesterase with respect to motor neurons; or (4) decreased symptoms of neuron dysfunction in vivo. Such effects may be measured by any method known in the art. In preferred, non-limiting embodiments, increased survival of neurons may routinely be measured using a method set forth herein or otherwise known in the art, such as, for example, the method set forth in Arakawa et al. (J. Neurosci. 10:3507-3515 (1990)); increased sprouting of neurons may be detected by methods known in the art, such as, for example, the methods set forth in Pestronk et al. (Exp. Neurol. 70:65-82 (1980)) or Brown et al. (Ann. Rev. Neurosci. 4:17-42 (1981)); increased production of neuron-associated molecules may be measured by bioassay, enzymatic assay, antibody binding, Northern blot assay, etc., using techniques known in the art and depending on the molecule to be measured; and motor neuron dysfunction may be measured by assessing the physical manifestation of motor neuron disorder, e.g., weakness, motor neuron conduction velocity, or functional disability.

In specific embodiments, motor neuron diseases, disorders, and/or conditions that may be treated, prevented, and/or diagnosed according to the invention include, but are not limited to, diseases, disorders, and/or conditions such as infarction, infection, exposure to toxin, trauma, surgical damage, degenerative disease or malignancy that may affect motor neurons as well as other components of the nervous system, as well as diseases, disorders, and/or conditions that selectively affect neurons such as amyotrophic lateral sclerosis, and including, but not limited to, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

In one embodiment in accordance with the present invention, the novel BMSOTR protein may play a role in neoplastic disorders. An antagonist or inhibitor of the BMSOTR protein may be administered to an individual to prevent or treat a neoplastic disorder. Such disorders may include, but are not limited to, adenocarcinoma, leukemia, lymphoma, melanoma, myeloma, sarcoma, and teratocarcinoma, and particularly, cancers of the adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid, and uterus. In a related aspect, an antibody which specifically binds to BMSOTR may be used directly as an antagonist or indirectly as a targeting or delivery mechanism for bringing a pharmaceutical agent to cells or tissue which express the BMSOTR polypeptide.

In yet another embodiment of the present invention, an antagonist or inhibitory agent of the BMSOTR polypeptide may be administered therapeutically to an individual to prevent or treat an immunological disorder. Such disorders may include, but are not limited to, AIDS, HIV infection, Addison's disease, adult respiratory distress syndrome, allergies, anemia, asthma, atherosclerosis, bronchitis, cholecystitis, Crohn's disease, ulcerative colitis, atopic dermatitis, dermatomyositis, diabetes mellitus, emphysema, erythema nodosum, atrophic gastritis, glomerulonephritis, gout, Graves' disease, hypereosinophilia, irritable bowel syndrome, lupus erythematosus, multiple sclerosis, myasthenia gravis, myocardial or pericardial inflammation, osteoarthritis, osteoporosis, pancreatitis, polymyositis, rheumatoid arthritis, scleroderma, Sjogren's syndrome, and autoimmune thyroiditis; complications of cancer, hemodialysis, extracorporeal circulation; viral, bacterial, fungal, parasitic, protozoal, and helminthic infections, trauma, and neurological disorders including, but not limited to, akathesia, Alzheimer's disease, amnesia, amyotrophic lateral sclerosis, bipolar disorder, catatonia, cerebral neoplasms, dementia, depression, Down's syndrome, tardive dyskinesia, dystonias, epilepsy, Huntington's disease, multiple sclerosis, Parkinson's disease, paranoid psychoses, schizophrenia, obsessive-compulsive disorder, Prader-Willi syndrome and Tourette's disorder.

A preferred method of treating a BMSOTR associated disease, disorder, syndrome, or condition in a mammal comprises administration of a modulator, preferably an inhibitor or antagonist, of a BMSOTR polypeptide or homologue of the invention, in an amount effective to treat, reduce, and/or ameliorate the symptoms incurred by the BMSOTR-associated disease, disorder, syndrome, or condition. In some instances, an agonist or enhancer of a BMSOTR polypeptide or homologue of the invention is administered in an amount effective to treat and/or ameliorate the symptoms incurred by a BMSOTR-related disease, disorder, syndrome, or condition. In other instances, the administration of a novel BMSOTR polypeptide or homologue thereof pursuant to the present invention is envisioned for administration to treat a BMSOTR associated disease.

In yet another embodiment of the present invention, an expression vector containing the complement of the polynucleotide encoding a BMSOTR polypeptide is administered to an individual to treat or prevent any one of the types of diseases, disorders, or conditions previously described, in an antisense therapy method.

The BMSOTR protein, modulators, including antagonists, antibodies, and agonists, complementary sequences, or vectors of the present invention can also be administered in combination with other appropriate therapeutic agents as necessary or desired. Selection of the appropriate agents for use in combination therapy may be made by the skilled practitioner in the art, according to conventional pharmaceutical and clinical principles. The combination of therapeutic agents may act synergistically to effect the treatment or prevention of the various disorders described above. Using this approach, one may be able to achieve therapeutic efficacy with lower dosages of each agent, thus reducing the potential for adverse side effects or adverse events.

The BMSOTR protein, modulators, including antagonists, antibodies, and agonists, complementary sequences, or vectors of the present invention can also be administered to act as taste modifiers.

Antagonists or inhibitors of the BMSOTR polypeptide of this invention can be produced using methods which are generally known in the art. In particular, purified BMSOTR protein, or fragments thereof, can be used to produce antibodies, or to screen libraries of pharmaceutical agents, to identify those which specifically bind to the novel BMSOTR polypeptide as described herein.

Antibodies specific for BMSOTR polypeptide, or immunogenic peptide fragments thereof, can be generated using methods that have long been known and conventionally practiced in the art. Such antibodies may include, but are not limited to, polyclonal, monoclonal, neutralizing antibodies, (i.e., those which inhibit dimer formation), chimeric, single chain, Fab fragments, and fragments produced by an Fab expression library. A non-limiting example of the BMSOTR polypeptide or immunogenic fragments thereof that may be used to generate antibodies is provided in SEQ ID NO:2.

For the production of antibodies, various hosts, including goats, rabbits, sheep, rats, mice, humans, and others, can be immunized by injection with the BMSOTR polypeptide, or any immunogenic and/or epitope-containing fragment or oligopeptide thereof, which have immunogenic properties. Depending on the host species, various adjuvants may be used to increase the immunological response. Non-limiting examples of suitable adjuvants include Freund's (incomplete), mineral gels such as aluminum hydroxide or silica, and surface active substances such as lysolecithin, pluronic polyols, polyanions, peptides, oil emulsions, KLH, and dinitrophenol. Adjuvants typically used in humans include BCG (bacilli Calmette Guérin) and Corynebacterium parvumn.

Preferably, the BMSOTR polypeptide, peptides, fragments, or oligopeptides used to induce antibodies to the BMSOTR polypeptide immunogens have an amino acid sequence of at least five amino acids in length, and more preferably, at least 7-10, or more, amino acids. It is also preferable that the immunogens are identical to a portion of the amino acid sequence of the natural protein; they may also contain the entire amino acid sequence of a small, naturally occurring molecule. The peptides, fragments or oligopeptides may comprise a single epitope or antigenic determinant or multiple epitopes. Short stretches of BMSOTR amino acids may be fused with another protein as carrier, such as KLH, such that antibodies are produced against the chimeric molecule.

Monoclonal antibodies to the BMSOTR polypeptide, or immunogenic fragments thereof, may be prepared using any technique which provides for the production of antibody molecules by continuous cell lines in culture. Such techniques are conventionally used in the art. These include, but are not limited to, the hybridoma technique, the human B-cell hybridoma technique, and the EBV-hybridoma technique (G. Köhler et al., 1975, Nature, 256:495-497; D. Kozbor et al., 1985, J. Immunol. Methods, 81:31-42; R. J. Cote et al., 1983, Proc. Natl. Acad. Sci. USA, 80:2026-2030; and S. P. Cole et al., 1984, Mol. Cell Biol., 62:109-120). The production of monoclonal antibodies to immunogenic proteins and peptides is well known and routinely used in the art.

In addition, techniques developed for the production of “chimeric antibodies,” the splicing of mouse antibody genes to human antibody genes to obtain a molecule with appropriate antigen specificity and biological activity can be used (S. L. Morrison et al., 1984, Proc. Natl. Acad. Sci. USA, 81:6851-6855; M. S. Neuberger et al., 1984, Nature, 312:604-608; and S. Takeda et al., 1985, Nature, 314:452-454). Alternatively, techniques described for the production of single chain antibodies may be adapted, using methods known in the art, to produce BMSOTR polypeptide-specific single chain antibodies. Antibodies with related specificity, but of distinct idiotypic composition, may be generated by chain shuffling from random combinatorial immunoglobulin libraries (D. R. Burton, 1991, Proc. Natl. Acad. Sci. USA, 88:11120-3). Antibodies may also be produced by inducing in vivo production in the lymphocyte population or by screening recombinant immunoglobulin libraries or panels of highly specific binding reagents as disclosed in the literature (R. Orlandi et al., 1989, Proc. Natl. Acad. Sci. USA, 86:3833-3837 and G. Winter et al., 1991, Nature, 349:293-299).

Antibody fragments, which contain specific binding sites for a BMSOTR polypeptide, may also be generated. For example, such fragments include, but are not limited to, F(ab′)₂ fragments which can be produced by pepsin digestion of the antibody molecule and Fab fragments which can be generated by reducing the disulfide bridges of the F(ab′)₂ fragments. Alternatively, Fab expression libraries may be constructed to allow rapid and easy identification of monoclonal Fab fragments with the desired specificity (e.g., W. D. Huse et al., 1989, Science, 254.1275-1281).

Various immunoassays can be used for screening to identify antibodies having the desired specificity. Numerous protocols for competitive binding or immunoradiometric assays using either polyclonal or monoclonal antibodies with established specificities are well known in the art. Such immunoassays typically involve measuring the formation of complexes between a BMSOTR polypeptide and its specific antibody. A two-site, monoclonal-based immunoassay utilizing monoclonal antibodies reactive with two non-interfering BMSOTR polypeptide epitopes is suitable, but a competitive binding assay may also be employed (Maddox, supra).

To induce an immunological response in a mammal, a host animal is inoculated with a BMSOTR polypeptide, or a fragment thereof, of this invention in an amount adequate to produce an antibody and/or a T cell immune response to protect the animal from a disease or disorder associated with the expression or production of a BMSOTR polypeptide. Yet another aspect of the invention relates to a method of inducing immunological response in a mammal, if applicable or required. Such a method comprises delivering BMSOTR polypeptide via a vector directing expression of BMSOTR polynucleotide in vivo in order to induce such an immunological response to produce antibody to protect said animal from BMSOTR-related diseases.

A further aspect of the invention relates to an immunological vaccine or immunogen formulation or composition which, when introduced into a mammalian host, induces an immunological response in that mammal to a BMSOTR polypeptide wherein the composition comprises a BMSOTR polypeptide or BMSOTR gene. The vaccine or immunogen formulation may further comprise a suitable carrier. Since the BMSOTR polypeptide may be broken down in the stomach, it is preferably administered parenterally (including subcutaneous, intramuscular, intravenous, intradermal, etc., injection). Formulations suitable for parenteral administration include aqueous and non-aqueous sterile injection solutions which may contain antioxidants, buffers, bacteriostats and solutes which render the formulation isotonic with the blood of the recipient; and aqueous and non-aqueous sterile suspensions which may include suspending agents or thickening agents.

The formulations may be presented in unit-dose or multi-dose containers, for example, sealed ampoules and vials, and may be stored in a freeze-dried condition requiring only the addition of the sterile liquid carrier immediately prior to use. A vaccine formulation may also include adjuvant systems for enhancing the immunogenicity of the formulation, such as oil-in-water systems and other systems known in the art. The dosage will depend on the specific activity of the vaccine and can be readily determined by routine experimentation.

In an aspect of the present invention, the polynucleotide encoding a BMSOTR polypeptide, or any fragment or complement thereof, as described herein may be used for therapeutic purposes. For instance, antisense to a BMSOTR polynucleotide encoding a BMSOTR polypeptide, may be used in situations in which it would be desirable to block the transcription of BMSOTR mRNA. In particular, cells may be transformed, transfected, or injected with sequences complementary to polynucleotides encoding BMSOTR polypeptide. Thus, complementary molecules may be used to modulate BMSOTR polynucleotide and polypeptide activity, or to achieve regulation of gene function. Such technology is well known in the art, and sense or antisense oligomers or oligonucleotides, or larger fragments, can be designed from various locations along the coding or control regions of the BMSOTR polynucleotide sequence encoding the novel BMSOTR polypeptide.

Polypeptides used in treatment can also be generated endogenously in the subject, in treatment modalities often referred to as “gene therapy”. Thus for example, cells from a subject may be engineered with a polynucleotide, such as DNA or RNA, to encode a polypeptide ex vivo, for example, by the use of a retroviral plasmid vector. The cells can then be introduced into the subject's body in which the desired polypeptide is expressed.

A gene encoding a BMSOTR polypeptide can be turned off by transforming a cell or tissue with an expression vector that expresses high levels of a BMSOTR polypeptide-encoding polynucleotide, or a fragment thereof. Such constructs may be used to introduce untranslatable sense or antisense sequences into a cell. Even in the absence of integration into the DNA, such vectors may continue to transcribe RNA molecules until they are disabled by endogenous nucleases. Transient expression may last for a month or more with a non-replicating vector, and even longer if appropriate replication elements are designed to be part of the vector system.

Modifications of gene expression can be obtained by designing antisense molecules or complementary nucleic acid sequences (DNA, RNA, or PNA), to the control, 5′, or regulatory regions of a BMSOTR polynucleotide sequence encoding a BMSOTR polypeptide, (e.g., a signal sequence, promoters, enhancers, and introns). Oligonucleotides may be derived from the transcription initiation site, for example, between positions −10 and +10 from the start site.

Similarly, inhibition can be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described (see, for example, J. E. Gee et al., 1994, In: B. E. Huber and B. I. Carr, Molecular and Immunologic Approaches, Futura Publishing Co., Mt. Kisco, N.Y.). The antisense molecule or complementary sequence may also be designed to block translation of mRNA by preventing the transcript from binding to ribosomes.

Many methods for introducing vectors into cells or tissues are available and are equally suitable for use in vivo, in vitro, and ex vivo. For ex vivo therapy, vectors may be introduced into stem cells or bone marrow cells obtained from the patient and clonally propagated for autologous transplant back into that same patient. Delivery by transfection, direct injection (e.g., microparticle bombardment) and by liposome injections may be achieved using methods which are well known in the art.

Any of the therapeutic methods described above can be applied to any individual in need of such therapy, including, for example, mammals such as dogs, cats, cows, horses, rabbits, monkeys, and most preferably, humans.

A polypeptide of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, organ or body part size or shape (such as, for example, breast augmentation or diminution, change in bone form or shape); effecting biorhythms or circadian cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors, analgesic effects or other pain reducing effects; promoting differentiation and growth of embryonic stem cells in lineages other than hernatopoletic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Polypeptide or polynucleotides and/or agonist or antagonists of the present invention may also be used to prepare individuals for extraterrestrial travel, low gravity environments, prolonged exposure to extraterrestrial radiation levels, low oxygen levels, reduction of metabolic activity, exposure to extraterrestrial pathogens, etc. Such a use may be administered either prior to an extraterrestrial event, during an extraterrestrial event, or both. Moreover, such a use may result in a number of beneficial changes in the recipient, such as, for example, any one of the following, non-limiting, effects: an increased level of hematopoietic cells, particularly red blood cells which would aid the recipient in coping with low oxygen levels; an increased level of B-cells, T-cells, antigen presenting cells, and/or macrophages, which would aid the recipient in coping with exposure to extraterrestrial pathogens, for example; a temporary (i.e., reversible) inhibition of hematopoietic cell production which would aid the recipient in coping with exposure to extraterrestrial radiation levels; increase and/or stability of bone mass which would aid the recipient in coping with low gravity environments; and/or decreased metabolism which would effectively facilitate the recipients ability to prolong their extraterrestrial travel by any one of the following, non-limiting means: (i) aid the recipient by decreasing their basal daily energy requirements; (ii) effectively lower the level of oxidative and/or metabolic stress in recipient (i.e., to enable recipient to cope with increased extraterrestial radiation levels by decreasing the level of internal oxidative/metabolic damage acquired during normal basal energy requirements; and/or (iii) enabling recipient to subsist at a lower metabolic temperature (i.e., cryogenic, and/or sub-cryogenic environment).

Polypeptide or polynucleotides and/or agonist or antagonists of the present invention may also be used to increase the efficacy of a pharmaceutical composition, either directly or indirectly. Such a use may be administered in simultaneous conjunction with said pharmaceutical, or separately through either the same or different route of administration (e.g., intravenous for the polynucleotide or polypeptide of the present invention, and orally for the pharmaceutical, among others described herein.).

### Pharmaceutical Preparations

A further embodiment of the present invention embraces the administration of a pharmaceutical composition, in conjunction with a pharmaceutically acceptable carrier, diluent, or excipient, to achieve any of the above-described therapeutic uses and effects. Depending upon the disease treatment, such pharmaceutical compositions can comprise BMSOTR nucleic acid, polypeptide, or peptides, antibodies to BMSOTR polypeptide, mimetics, BMSOTR modulators, such as agonists, antagonists, or inhibitors of a BMSOTR polypeptide or polynucleotide. The compositions can comprise the active agent or ingredient alone, or in combination with at least one other agent or reagent, such as a stabilizing compound, which may be administered in any sterile, biocompatible pharmaceutical carrier, including, but not limited to, saline, buffered saline, dextrose, and water. The compositions may be administered to a patient alone, or in combination with other agents, drugs, hormones, or biological response modifiers.

The pharmaceutical compositions for use in the present invention can be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, vaginal, or rectal means.

In addition to the active ingredients (e.g., BMSOTR nucleic acid or polypeptide, or functional fragments thereof, or a BMSOTR agonist or antagonist), the pharmaceutical compositions may contain pharmaceutically acceptable/physiologically suitable carriers or excipients comprising auxiliaries which facilitate processing of the active compounds into preparations that can be used pharmaceutically. Further details on techniques for formulation and administration are provided in the latest edition of Remington's Pharmaceutical Sciences (Mack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

In addition, pharmaceutical preparations for oral use can be obtained by the combination of active compounds with a solid excipient, optionally grinding a resulting mixture, and processing the mixture of granules, after adding suitable auxiliaries, if desired, to obtain tablets or dragee cores. Suitable excipients are carbohydrate or protein fillers, such as sugars, including lactose, sucrose, mannitol, or sorbitol; starch from corn, wheat, rice, potato, or other plants; cellulose, such as methyl cellulose, hydroxypropyl-methylcellulose, or sodium carboxymethylcellulose; gums, including arabic and tragacanth, and proteins such as gelatin and collagen. If desired, disintegrating or solubilizing agents may be added, such as cross-linked polyvinyl pyrrolidone, agar, alginic acid, or a physiologically acceptable salt thereof, such as sodium alginate.

Dragee cores may be used in conjunction with physiologically suitable coatings, such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification, or to characterize the quantity of active compound, i.e., dosage.

Pharmaceutical preparations, which can be used orally, further include push-fit capsules made of gelatin, as well as soft, scaled capsules made of gelatin and a coating, such as glycerol or sorbitol. Push-fit capsules can contain active ingredients mixed with fillers or binders, such as lactose or starches, lubricants, such as talc or magnesium stearate, and, optionally, stabilizers. In soft capsules, the active compounds may be dissolved or suspended in suitable liquids, such as fatty oils, liquid, or liquid polyethylene glycol with or without stabilizers.

Pharmaceutical formulations suitable for parenteral administration may be formulated in aqueous solutions, preferably in physiologically compatible buffers such as Hanks' solution, Ringer's solution, or physiologically buffered saline. Aqueous injection suspensions may contain substances, which increase the viscosity of the suspension, such as sodium carboxymethyl cellulose, sorbitol, or dextran. In addition, suspensions of the active compounds may be prepared as appropriate oily injection suspensions. Suitable lipophilic solvents or vehicles include fatty oils such as sesame oil, or synthetic fatty acid esters, such as ethyloleate or triglycerides, or liposomes. Optionally, the suspension may also contain suitable stabilizers or agents which increase the solubility of the compounds to allow for the preparation of highly concentrated solutions.

For topical or nasal administration, penetrants or permeation agents (enhancers) that are appropriate to the particular barrier to be permeated are used in the formulation. Such penetrants are generally known in the art.

The pharmaceutical compositions of the present invention can be manufactured in a manner that is known in the art, e.g., by means of conventional mixing, dissolving, granulating, dragee-making, levigating, emulsifying, encapsulating, entrapping, or lyophilizing processes.

A pharmaceutical composition can be provided as a salt and can be formed with many acids, including but not limited to, hydrochloric, sulfuric, acetic, lactic, tartaric, malic, succinic, and the like. Salts tend to be more soluble in aqueous solvents, or other protonic solvents, than are the corresponding free base forms. In other cases, the preferred preparation may be a lyophilized powder which may contain any or all of the following: 1-50 mM histidine, 0.1%-2% sucrose, and 2-7% mannitol, at a pH range of 4.5 to 5.5, combined with a buffer prior to use. After the pharmaceutical compositions have been prepared, they can be placed in an appropriate container and labeled for treatment of an indicated condition. For administration of a BMSOTR product, such labeling would include amount, frequency, and method of administration.

Pharmaceutical compositions suitable for use in the present invention include compositions wherein the active ingredients are contained in an effective amount to achieve the intended purpose. The determination of an effective dose or amount is well within the capability of those skilled in the art. For any compound, the therapeutically effective dose can be estimated initially either in cell culture assays, for example, using neoplastic cells, or in animal models, usually mice, rabbits, dogs, or pigs. The animal model may also be used to determine the appropriate concentration range and route of administration. Such information can then be used and extrapolated to determine useful doses and routes for administration in humans.

A therapeutically effective dose refers to that amount of active ingredient, for example, BMSOTR polynucleotide, BMSOTR polypeptide, or fragments thereof, antibodies to BMSOTR polypeptide, agonists, antagonists or inhibitors of BMSOTR polypeptide, which ameliorates, reduces, diminishes, or eliminates the symptoms or condition. Therapeutic efficacy and toxicity can be determined by standard pharmaceutical procedures in cell cultures or in experimental animals, e.g., ED₅₀ (the dose therapeutically effective in 50% of the population) and LD₅₀ (the dose lethal to 50% of the population). The dose ratio of toxic to therapeutic effects is the therapeutic index, which can be expressed as the ratio, LD₅₀/ED₅₀. Pharmaceutical compositions which exhibit large therapeutic indices are preferred. The data obtained from cell culture assays and animal studies are used in determining a range of dosages for human use. Preferred dosage contained in a pharmaceutical composition is within a range of circulating concentrations that include the ED₅₀ with little or no toxicity. The dosage varies within this range depending upon the dosage form employed, the sensitivity of the patient, and the route of administration.

The practitioner, who will consider the factors related to an individual requiring treatment, will determine the exact dosage. Dosage and administration are adjusted to provide sufficient levels of the active component, or to maintain the desired effect. Factors which may be taken into account include the severity of the individual's disease state; the general health of the patient; the age, weight, and gender of the patient; diet; time and frequency of administration; drug combination(s); reaction sensitivities; and tolerance/response to therapy. As a general guide, long-acting pharmaceutical compositions may be administered every 3 to 4 days, every week, or once every two weeks, depending on half-life and clearance rate of the particular formulation. Variations in these dosage levels can be adjusted using standard empirical routines for optimization, as is well understood in the art.

As a guide, normal dosage amounts may vary from 0.1 to 100,000 micrograms (μg), up to a total dose of about 1 gram (g), depending upon the route of administration. Guidance as to particular dosages and methods of delivery is provided in the literature and is generally available to practitioners in the art. Those skilled in the art will employ different formulations for nucleotides than for proteins or their inhibitors or activators. Similarly, the delivery of polynucleotides or polypeptides will be specific to particular cells, conditions, locations, and the like.

### Microarrays and Screening Assays

In another embodiment of the present invention, oligonucleotides, or longer fragments derived from the BMSOTR polynucleotide sequence described herein can be used as targets in a microarray. The microarray can be used to monitor the expression levels of large numbers of genes simultaneously (to produce a transcript image), and to identify genetic variants, mutations and polymorphisms. This information may be used to determine gene function, to understand the genetic basis of a disease, to diagnose disease, and to develop and monitor the activities of therapeutic agents. In a particular aspect, the microarray is prepared and used according to the methods described in WO 95/11995 (Chee et al.); D. J. Lockhart et al., 1996, Nature Biotechnology, 14:1675-1680; and M. Schena et al., 1996, Proc. Natl. Acad. Sci. USA, 93:10614-10619). Microarrays are further described in U.S. Pat. No. 6,015,702 to P. Lal et al.

In another embodiment of this invention, a nucleic acid sequence which encodes a novel BMSOTR polypeptide, may also be used to generate hybridization probes, which are useful for mapping the naturally occurring genomic sequence. The sequences may be mapped to a particular chromosome, to a specific region of a chromosome, or to artificial chromosome constructions (HACs), yeast artificial chromosomes (YACs), bacterial artificial chromosomes (BACs), bacterial PI constructions, or single chromosome cDNA libraries, as reviewed by C. M. Price, 1993, Blood Rev., 7:127-134 and by B. J. Trask, 1991, Trends Genet., 7:149-154.

In another embodiment of the present invention, a BMSOTR polypeptide of this invention, its catalytic or immunogenic fragments, or oligopeptides thereof, can be used for screening libraries of compounds in any of a variety of drug screening techniques. The fragment employed in such screening may be free in solution, affixed to a solid support, borne on a cell surface, or located intracellularly. The formation of binding complexes, between the BMSOTR polypeptide, or a portion thereof, and the agent being tested, may be measured utilizing techniques commonly practiced in the art.

Another technique for drug screening, which may be employed, provides for high throughput screening of compounds having suitable binding affinity to the protein of interest as described in WO 84/03564 (Venton, et al.). In this method, as applied to the BMSOTR protein, large numbers of different small test compounds are synthesized on a solid substrate, such as plastic pins or some other surface. The test compounds are reacted with the BMSOTR polypeptide, or fragments thereof, and washed. Bound BMSOTR polypeptide is then detected by methods well known in the art. Purified BMSOTR polypeptide can also be coated directly onto plates for use in the aforementioned drug screening techniques. Alternatively, non-neutralizing antibodies can be used to capture the peptide and immobilize it on a solid support.

In a further embodiment, competitive drug screening assays can be used in which neutralizing antibodies, capable of binding a BMSOTR polypeptide according to this invention, specifically compete with a test compound for binding to the BMSOTR polypeptide. In this manner, the antibodies can be used to detect the presence of any peptide that shares one or more antigenic determinants with the BMSOTR polypeptide.

The human BMSOTR and/or BMSORT variant polypeptides and/or peptides of the present invention, or immunogenic fragments or oligopeptides thereof, can be used for screening therapeutic drugs or compounds in a variety of drug screening techniques. The fragment employed in such a screening assay may be free in solution, affixed to a solid support, borne on a cell surface, or located intracellularly. The reduction or abolition of activity of the formation of binding complexes between the ion channel protein and the agent being tested can be measured. Thus, the present invention provides a method for screening or assessing a plurality of compounds for their specific binding affinity with a BMSOTR and/or BMSORT variant polypeptide, or a bindable peptide fragment, of this invention, comprising providing a plurality of compounds, combining the BMSOTR and/or BMSORT variant polypeptide, or a bindable peptide fragment, with each of a plurality of compounds for a time sufficient to allow binding under suitable conditions and detecting binding of the BMSOTR and/or BMSORT variant polypeptide or peptide to each of the plurality of test compounds, thereby identifying the compounds that specifically bind to the BMSOTR and/or BMSORT variant polypeptide or peptide.

Methods of identifying compounds that modulate the activity of the novel human BMSOTR and/or BMSORT variant polypeptides and/or peptides are provided by the present invention and comprise combining a potential or candidate compound or drug modulator of GPCR biological activity with an BMSOTR and/or BMSORT variant polypeptide or peptide, for example, the BMSOTR and/or BMSORT variant amino acid sequence as set forth in SEQ ID NO:2, and measuring an effect of the candidate compound or drug modulator on the biological activity of the BMSOTR and/or BMSORT variant polypeptide or peptide. Such measurable effects include, for example, physical binding interaction; the ability to cleave a suitable GPCR substrate; effects on native and cloned BMSOTR and/or BMSORT variant-expressing cell line; and effects of modulators or other GPCR-mediated physiological measures.

Another method of identifying compounds that modulate the biological activity of the novel BMSOTR and/or BMSORT variant polypeptides of the present invention comprises combining a potential or candidate compound or drug modulator of a GPCR biological activity with a host cell that expresses the BMSOTR and/or BMSORT variant polypeptide and measuring an effect of the candidate compound or drug modulator on the biological activity of the BMSOTR and/or BMSORT variant polypeptide. The host cell can also be capable of being induced to express the BMSOTR and/or BMSORT variant polypeptide, e.g., via inducible expression. Physiological effects of a given modulator candidate on the BMSOTR and/or BMSORT variant polypeptide can also be measured. Thus, cellular assays for particular GPCR modulators may be either direct measurement or quantification of the physical biological activity of the BMSOTR and/or BMSORT variant polypeptide, or they may be measurement or quantification of a physiological effect. Such methods preferably employ a BMSOTR and/or BMSORT variant polypeptide as described herein, or an overexpressed recombinant BMSOTR and/or BMSORT variant polypeptide in suitable host cells containing an expression vector as described herein, wherein the BMSOTR and/or BMSORT variant polypeptide is expressed, overexpressed, or undergoes upregulated expression.

Another aspect of the present invention embraces a method of screening for a compound that is capable of modulating the biological activity of a BMSOTR and/or BMSORT variant polypeptide, comprising providing a host cell containing an expression vector harboring a nucleic acid sequence encoding a BMSOTR and/or BMSORT variant polypeptide, or a functional peptide or portion thereof (e.g., SEQ ID NOS:2); determining the biological activity of the expressed BMSOTR and/or BMSORT variant polypeptide in the absence of a modulator compound; contacting the cell with the modulator compound and determining the biological activity of the expressed BMSOTR and/or BMSORT variant polypeptide in the presence of the modulator compound. In such a method, a difference between the activity of the BMSOTR and/or BMSORT variant polypeptide in the presence of the modulator compound and in the absence of the modulator compound indicates a modulating effect of the compound.

Essentially any chemical compound can be employed as a potential modulator or ligand in the assays according to the present invention. Compounds tested as GPCR modulators can be any small chemical compound, or biological entity (e.g., protein, sugar, nucleic acid, lipid). Test compounds will typically be small chemical molecules and peptides. Generally, the compounds used as potential modulators can be dissolved in aqueous or organic (e.g., DMSO-based) solutions. The assays are designed to screen large chemical libraries by automating the assay steps and providing compounds from any convenient source. Assays are typically run in parallel, for example, in microtiter formats on microtiter plates in robotic assays. There are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland), for example. Also, compounds may be synthesized by methods known in the art.

High throughput screening methodologies are particularly envisioned for the detection of modulators of the novel BMSOTR and/or BMSORT variant polynucleotides and polypeptides described herein. Such high throughput screening methods typically involve providing a combinatorial chemical or peptide library containing a large number of potential therapeutic compounds (e.g., ligand or modulator compounds). Such combinatorial chemical libraries or ligand libraries are then screened in one or more assays to identify those library members (e.g., particular chemical species or subclasses) that display a desired characteristic activity. The compounds so identified can serve as conventional lead compounds, or can themselves be used as potential or actual therapeutics.

A combinatorial chemical library is a collection of diverse chemical compounds generated either by chemical synthesis or biological synthesis, by combining a number of chemical building blocks (i.e., reagents such as amino acids). As an example, a linear combinatorial library, e.g., a polypeptide or peptide library, is formed by combining a set of chemical building blocks in every possible way for a given compound length (i.e., the number of amino acids in a polypeptide or peptide compound). Millions of chemical compounds can be synthesized through such combinatorial mixing of chemical building blocks.

The preparation and screening of combinatorial chemical libraries is well known to those having skill in the pertinent art. Combinatorial libraries include, without limitation, peptide libraries (e.g. U.S. Pat. No. 5,010,175; Furka, 1991, Int. J. Pept. Prot. Res., 37:487-493; and Houghton et al., 1991, Nature, 354:84-88). Other chemistries for generating chemical diversity libraries can also be used. Nonlimiting examples of chemical diversity library chemistries include, peptides (PCT Publication No. WO 91/019735), encoded peptides (PCT Publication No. WO 93/20242), random bio-oligomers (PCT Publication No. WO 92/00091), benzodiazepines (U.S. Pat. No. 5,288,514), diversomers such as hydantoins, benzodiazepines and dipeptides (Hobbs et al., 1993, Proc. Natl. Acad. Sci. USA, 90:6909-6913), vinylogous polypeptides (Hagihara et al., 1992, J. Amer. Chem. Soc., 114:6568), nonpeptidal peptidomimetics with glucose scaffolding (Hirschmann et al., 1992, J. Amer. Chem. Soc., 114:9217-9218), analogous organic synthesis of small compound libraries (Chen et al., 1994, J. Amer. Chem. Soc., 116:2661), oligocarbamates (Cho et al., 1993, Science, 261:1303), and/or peptidyl phosphonates (Campbell et al., 1994, J. Org. Chem., 59:658), nucleic acid libraries (see Ausubel, Berger and Sambrook, all supra), peptide nucleic acid libraries (U.S. Pat. No. 5,539,083), antibody libraries (e.g., Vaughn et al., 1996, Nature Biotechnology, 14(3):309-314) and PCT/US96/10287), carbohydrate libraries (e.g., Liang et al., 1996, Science, 274-1520-1522) and U.S. Pat. No. 5,593,853), small organic molecule libraries (e.g., benzodiazepines, Baum C&EN, Jan. 18, 1993, page 33; and U.S. Pat. No. 5,288,514; isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; and the like).

Devices for the preparation of combinatorial libraries are commercially available (e.g., 357 MPS, 390 MPS, Advanced Chem Tech, Louisville Ky.; Symphony, Rainin, Woburn, Mass.; 433A Applied Biosystems, Foster City, Calif.; 9050 Plus, Millipore, Bedford, Mass.). In addition, a large number of combinatorial libraries are commercially available (e.g., ComGenex, Princeton, N.J.; Asinex, Moscow, Russia; Tripos, Inc., St. Louis, Mo.; ChemStar, Ltd., Moscow, Russia; 3D Pharmaceuticals, Exton, Pa.; Martek Biosciences, Columbia, Md., and the like).

In one embodiment, the invention provides solid phase based in vitro assays in a high throughput format, where the cell or tissue expressing an ion channel is attached to a solid phase substrate. In such high throughput assays, it is possible to screen up to several thousand different modulators or ligands in a single day. In particular, each well of a microtiter plate can be used to perform a separate assay against a selected potential modulator, or, if concentration or incubation time effects are to be observed, every 5-10 wells can test a single modulator. Thus, a single standard microtiter plate can assay about 96 modulators. If 1536 well plates are used, then a single plate can easily assay from about 100 to about 1500 different compounds. It is possible to assay several different plates per day; thus, for example, assay screens for up to about 6,000-20,000 different compounds are possible using the described integrated systems.

In another of its aspects, the present invention encompasses screening and small molecule (e.g., drug) detection assays which involve the detection or identification of small molecules that can bind to a given protein, i.e., a BMSOTR and/or BMSORT variant polypeptide or peptide. Particularly preferred are assays suitable for high throughput screening methodologies.

In such binding-based detection, identification, or screening assays, a functional assay is not typically required. All that is needed is a target protein, preferably substantially purified, and a library or panel of compounds (e.g., ligands, drugs, small molecules) or biological entities to be screened or assayed for binding to the protein target. Preferably, most small molecules that bind to the target protein will modulate activity in some manner, due to preferential, higher affinity binding to functional areas or sites on the protein.

An example of such an assay is the fluorescence based thermal shift assay (3-Dimensional Pharmaceuticals, Inc., 3DP, Exton, Pa.) as described in U.S. Pat. Nos. 6,020,141 and 6,036,920 to Pantoliano et al.; see also, J. Zimmerman, 2000, Gen. Eng. News, 20(8)). The assay allows the detection of small molecules (e.g., drugs, ligands) that bind to expressed, and preferably purified, ion channel polypeptide based on affinity of binding determinations by analyzing thermal unfolding curves of protein-drug or ligand complexes. The drugs or binding molecules determined by this technique can be further assayed, if desired, by methods, such as those described herein, to determine if the molecules affect or modulate function or activity of the target protein.

To purify a BMSOTR and/or BMSORT variant polypeptide or peptide to measure a biological binding or ligand binding activity, the source may be a whole cell lysate that can be prepared by successive freeze-thaw cycles (e.g., one to three) in the presence of standard protease inhibitors. The BMSOTR and/or BMSORT variant polypeptide may be partially or completely purified by standard protein purification methods, e.g., affinity chromatography using specific antibody described infra, or by ligands specific for an epitope tag engineered into the recombinant BMSOTR and/or BMSORT variant polypeptide molecule, also as described herein. Binding activity can then be measured as described.

Compounds which are identified according to the methods provided herein, and which modulate or regulate the biological activity or physiology of the BMSOTR and/or BMSORT variant polypeptides according to the present invention are a preferred embodiment of this invention. It is contemplated that such modulatory compounds may be employed in treatment and therapeutic methods for treating a condition that is mediated by the novel BMSOTR and/or BMSORT variant polypeptides by administering to an individual in need of such treatment a therapeutically effective amount of the compound identified by the methods described herein.

In addition, the present invention provides methods for treating an individual in need of such treatment for a disease, disorder, or condition that is mediated by the BMSOTR and/or BMSORT variant polypeptides of the invention, comprising administering to the individual a therapeutically effective amount of the BMSOTR and/or BMSORT variant-modulating compound identified by a method provided herein.

### EXAMPLES

The Examples herein are meant to exemplify the various aspects of carrying out the invention and are not intended to limit the scope of the invention in any way. The Examples do not include detailed descriptions for conventional methods employed, such as in the construction of vectors, the insertion of cDNA into such vectors, or the introduction of the resulting vectors into the appropriate host. Such methods are well known to those skilled in the art and are described in numerous publications, for example, Sambrook, Fritsch, and Maniatis, Molecular Cloning: A Laboratory Manual, 2^{nd} Edition, Cold Spring Harbor Laboratory Press, USA, (1989).

### Example 1

### Bioinformatics Analysis

Currently, one approach used for identifying and characterizing the genes distributed along the human genome includes utilizing large fragments of genomic DNA which are isolated, cloned, and sequenced. Potential open reading frames in these genomic sequences were identified using bioinformatics software.

GPCR sequences were obtained from the GPCR database at European Molecular Biology Laboratory (EMBL) (http://www.7tm.org/gpcr/). These sequences (more than 1300 protein sequences) were used as probes to search the human genomic, public and private EST databases. The search program used was BLAST2.0 (S. F. Altschul et al., 1997, Nucl. Acid. Res., 25:3389-3402). The top genomic exon hits from the BLAST results were searched back against the non-redundant protein and patent sequence databases. From this analysis, exons encoding novel GPCR's were identified based on sequence homology. Also, the genomic region surrounding the matching exons was analyzed. Based on this analysis, the potential full length nucleotide sequence (SEQ ID NO:1, FIGS. 1A-B) of the novel human GPCR, BMSOTR, and a BMSOTR variant (SEQ ID NO:3, FIGS. 2A-B), were identified.

The amino acid sequence of the BMSOTR polypeptide (SEQ ID NO:2) encoded by the BMSOTR polynucleotide sequence (SEQ ID NO:1), and the amino acid sequence of the BMSOTR variant polypeptide (SEQ ID NO:4) encoded by the BMSOTR variant polynucleotide sequence (SEQ ID NO:3), were searched against profile Hidden Markov Models (HMM) of GPCRs. Profile hidden Markov models (profile HMMs) are built from the Pfam alignments. The Pfam is a database of multiple alignments of protein domains or conserved protein regions. The alignments represent some evolutionary conserved structure, which has implications for the protein's function. Profile HMMs are useful for automatically recognizing that a new protein belongs to an existing protein family. (Bateman et al. Nucleic Acids Research, 28:263-266 (2000)). Both BMSOTR and the BMSOTR variant matched significantly to the 7-transmembrane receptor Pfam model (rhodopsin GPCR family (SEQ ID NO:14)) (FIGS. 3A-B). In FIGS. 3A-B, the query amino acid sequence, ‘Q’, indicates the amino acid sequence of BMSOTR in FIG. 3A, and BMSOTR in FIG. 3B. The target amino acid sequence, ‘T’, indicates the amino acid sequence of the 7-transmembrane receptor Pfam model. Based upon this prediction, it is expected that the BMSOTR polypeptide and BMSOTR variant polypeptide share biological activity with members of the rhodopsin family of transmembrane receptors, in addition to specific members known in the art, or as otherwise described herein.

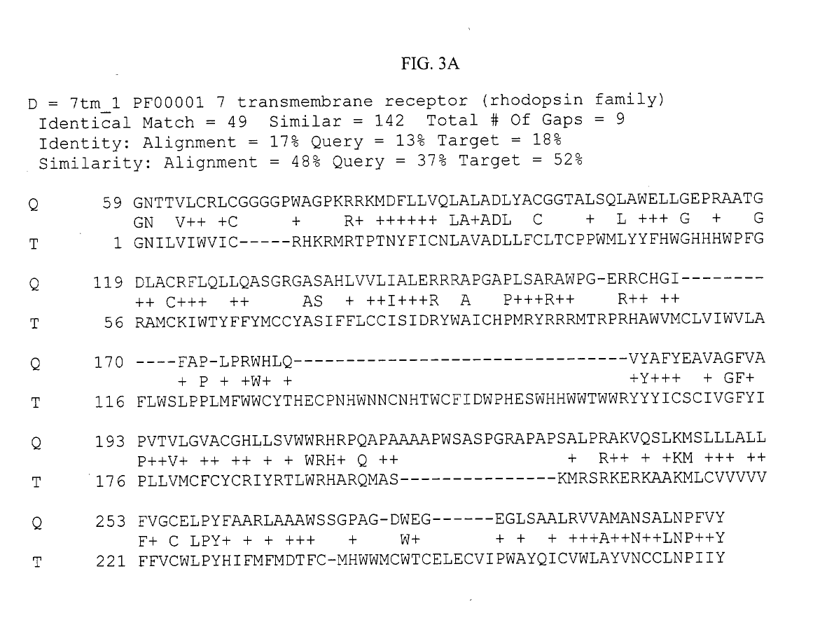

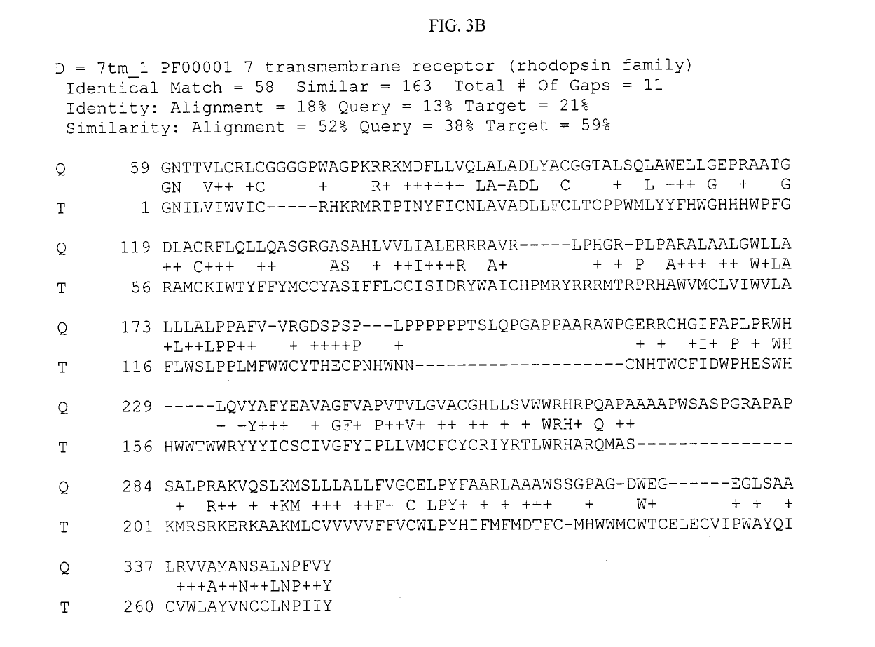

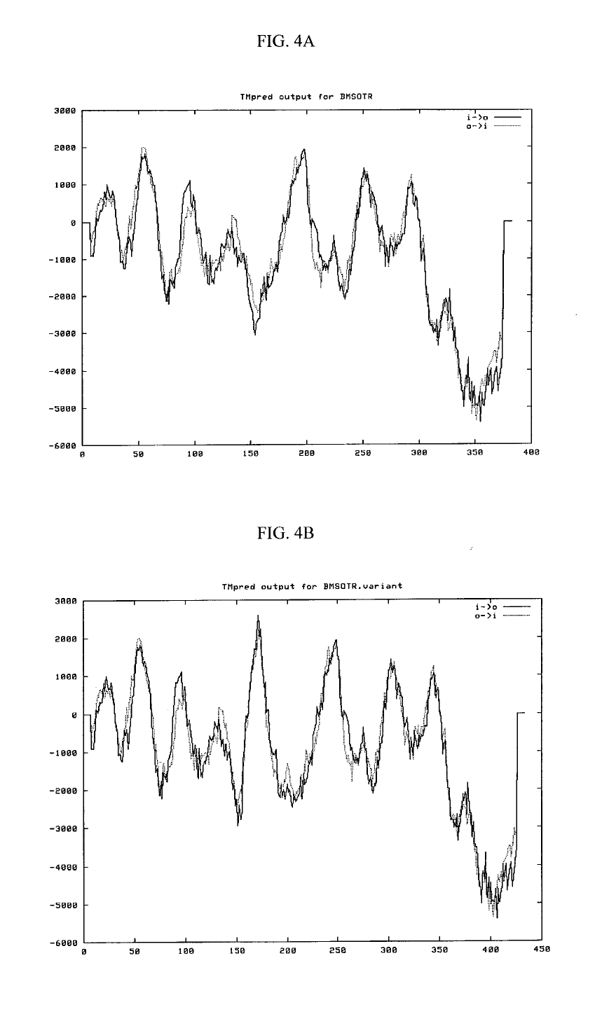

The amino acid sequences of the BMSOTR polypeptide and BMSOTR variant polypeptide were also analyzed for potential transmembrane domains. The TMPRED program, (Hofmann and Stoffel, Biol. Chem. Hoppe-Seyler, 347:166 (1993)), was used for transmembrane prediction. FIGS. 4A (BMSOTR) and 4B (BMSOTR variant) present a graphical representation of the prediction results. Each amino acid is given a score, residues that score above 500 are strongly considered to be a transmembrane residue. For example, in interpreting the results from FIG. 4A, the following amino acid regions of BMSOTR were highly predicted to be transmembrane helices (TM1 thru TM7): from about amino acid 13 to about amino acid 31, from about amino acid 44 to about amino acid 65, from about amino acid 84 to about amino acid 105, from about amino acid 186 to about amino acid 209, from about amino acid 240 to about amino acid 262, and from about amino acid 284 to about amino acid 304. In this context, the term “about” may be construed to mean 1, 2, 3, 4, 5, 6, 7, 8, 9, or 10 amino acids beyond the N-Terminus and/or C-terminus of the above referenced transmembrane domain polypeptides. Residues 126-145 was predicted to be a weak transmembrane helix. These results are consistent with the profile HMMs prediction, namely that the polypeptide sequence of BMSOTR contains seven transmembrane domains.

The present invention also encompasses the polypeptide sequences that intervene between each of the predicted BMSOTR transmembrane domains. Since these regions are solvent accessible either extracellularly or intracellularly, they are particularly useful for designing antibodies specific to each region. Such antibodies may be useful as antagonists or agonists of the BMSOTR full-length polypeptide and may modulate its activity.

In preferred embodiments, the present invention encompasses the use of N-terminal deletions, C-terminal deletions, or any combination of N-terminal and C-terminal deletions of any one or more of the BMSOTR TM1 thru TM7 transmembrane domain polypeptides as antigenic and/or immunogenic epitopes.

The following amino acid regions of BMSOTR variant were highly predicted to be transmembrane helices (TM1 thru TM7): from about amino acid 13 to about amino acid 31, from about amino acid 44 to about amino acid 65, from about amino acid 84 to about amino acid 105, from about amino acid 163 to about amino acid 183, from about amino acid 237 to about amino acid 260, from about amino acid 291 to about amino acid 313, and from about amino acid 335 to about amino acid 354. In this context, the term “about” may be construed to mean 1, 2, 3, 4, 5, 6, 7, 8, 9, or 10 amino acids beyond the N-Terminus and/or C-terminus of the above referenced transmembrane domain polypeptides. These results are consistent with the profile HMMs prediction, namely that the polypeptide sequence of BMSOTR variant contains seven transmembrane domains.

The present invention also encompasses the polypeptide sequences that intervene between each of the predicted BMSOTR variant transmembrane domains. Since these regions are solvent accessible either extracellularly or intracellularly, they are particularly useful for designing antibodies specific to each region. Such antibodies may be useful as antagonists or agonists of the BMSOTR variant full-length polypeptide and may modulate its activity.

In preferred embodiments, the present invention encompasses the use of N-terminal deletions, C-terminal deletions, or any combination of N-terminal and C-terminal deletions of any one or more of the BMSOTR variant TM1 thru TM7 transmembrane domain polypeptides as antigenic and/or immunogenic epitopes.

The amino acid sequence of the BMSOTR polypeptide (SEQ ID NO:2) encoded by the BMSOTR polynucleotide sequence (SEQ ID NO:1), and the amino acid sequence of the BMSOTR variant polypeptide (SEQ ID NO:4) encoded by the BMSOTR variant polynucleotide sequence (SEQ ID NO:3) were further searched using the BLAST2.0 program against the non-redundant protein and patent sequence databases. The alignment of BMSOTR (SEQ ID NO:2) and BMSOTR variant (SEQ ID NO:4) polypeptide sequences with the top matching hits was performed using the CLUSTALW algorithm using default parameters (S. F. Altschul, et al., Nucleic Acids Res. 25:3389-3402, 1997). See FIGS. 6A-B. These results indicate that the BMSOTR polypeptide of this invention represents a novel member of the GPCR protein super-family and vasopressin/oxytocin receptor GPCR sub-family. It is thus expected that the BMSOTR and BMSOTR variant polypeptides share biological activity with members of the GPCR family of transmembrane receptors, in addition to specific members known in the art, or as otherwise described herein.

The sequence information from the novel gene candidates is used for full-length cloning and expression profiling. Primer sequences are obtained using the primer3 program (Steve Rozen, Helen J. Skaletsky (1996, 1997) Primer3. Code available at http://www-genome.wi.mit.edu/genome_software/other/primer3.html). The right and left primers (SEQ ID NOS:5-6 and 8-9) are used in the cloning process and the “internal oligos” (SEQ ID NOS:7 and 10) are used as hybridization probes to detect the PCR product after amplification.

### Example 2

### Cloning of the Novel Human GPCR BMSOTR

The multiplex cloning method was devised as a means of extending large numbers of bioinformatic gene predictions into full length sequences by multiplexing probes and cDNA libraries in an effort to minimize the overall effort typically required for cDNA cloning. The method relies on the conversion of plasmid-based directionally cloned cDNA libraries into a population of pure, covalently-closed, circular, single-stranded molecules and long biotinylated DNA oligo probes designed from predicted gene sequences.

Probes and libraries are subjected to solution hybridization in a formamide buffer which has been found to be superior to aqueous buffers recommended in other biotin/strepavidin cDNA capture methods (i.e., Gene Trapper). The hybridization is set up without prior knowledge of the clones' representations in the libraries. The hybridization is carried out twice. After the first selection, the isolated sequences are screened with PCR primers specific for the targeted clones. The second hybridization is carried out with only those oligo probes whose gene-specific PCR assays gave positive results. The secondary hybridization serves to ‘normalize’ the selected library thereby decreasing the amount of screening needed to identify particular clones.

The method is robust and sensitive. Typically, dozens of cDNAs are isolated for any one particular gene, thereby increasing the chances of obtaining a full length cDNA. The entire complexity of any cDNA library is screened in the solution hybridization process, which is advantageous for finding rare sequences. The procedure is scaleable, with 50 oligo probes per experiment currently being used. In addition, it has been determined that larger number of probes can also be employed.

Using the bioinformatic predicted gene sequence, an antisense oligonucleotide with biotin on the 5′ end complementary to the putative coding region of BMSOTR is designed. This biotinylated oligo can be incubated with a mixture of single-stranded covalently closed circular cDNA libraries, which contain DNA corresponding to the sense strand. Hybrids between the biotinylated oligo and the circular cDNA are captured on streptavidin magnetic beads. Upon thermal release of the cDNA from the biotinylated oligo, the single stranded cDNA is converted into double strands using a primer homologous to a sequence on the cDNA cloning vector. The double stranded cDNA is introduced into E. coli by electroporation and the resulting colonies are screened by PCR, using a primer pair designed from the predicted gene sequence to identify the proper cDNA. Oligos used to identify the cDNA of the BMSOTR gene of this invention by PCR can be selected from the BMSOTR sequence as represented in SEQ ID NO:1.

### Example 3

### Multiplex Cloning of BMSOTR cDNA and BMSOTR Variant cDNA General Strategy

Using bioinformatic predicted gene sequence, the following types of gene-specific PCR primers and cloning oligos are designed: ‘A’ type PCR primer pairs that reside within a single predicted exon, ‘B’ type PCR primer pairs that cross putative exon/intron boundaries, and ‘C’ type, 80mer antisense and sense oligos containing a biotin moiety on its 5′ end. The primer pairs from the A type are optimized on human genomic DNA, and the B type on a mixture of first strand cDNAs made with and without reverse transcriptase, from brain and testis poly A+ RNA. The information obtained with the B type primers is used to assess which putative expressed sequences can be experimentally observed to have reverse transcriptase dependent expression. The primer pairs from the A type are less stringent in terms of identifying expressed sequences, but because they amplify genomic DNA as well as cDNA, the ability to amplify genomic DNA provides for the necessary positive control for the primer pair. Negative results with the B type are subjected to the caveat that the first strand sequence may not be expressed in the tissue that is under examination, and without a positive control, a negative result is meaningless.

The biotinylated 80 mer oligos are added en mass to pools of single strand cDNA libraries. Up to 50 probes have been successfully used on pools for 15 different libraries. The orientation of the oligo depends on the orientation of the cDNA in its vector. Antisense 80 mer oligos are used for those libraries and cloned into pCMVSPORT and pSPORT whereas sense 80 mer oligos are used for cDNA libraries cloned into pSPORT2. After the primary selection is carried out, all of the captured DNA is repaired to double strand form using the T7 primer for the commercial libraries in pCMVSPORT, and the Sp6 primer for in-house constructed libraries in pSPORT. The resulting DNA is electroporated into E. coli DH12 S and plated onto 150 mm plates with nylon filters. The cells are scraped and a frozen stock is made. This is the primary selected library. One-fifth of the library is generally converted into single strand form and the DNA assayed with the gene specific primers pairs (GSPs). The next round of solution hybridization capture is carried out with 80 mer oligos for only those sequences that were positive with the genes-specific-primers. After the second round, the captured single strand DNAs are repaired with a pool of GSPs, where only the primer complementary to polarity of the single-stranded circular DNA is used (the antisense primer for pCMVSPORT and pSPORT1 and the sense primer for pSPORT2). The resulting colonies are screened by PCR using the GSPs. Typically, greater than 80% of the clones are positive for any given GSP. The entire 96 well block of clones are min-prep and each of clones sized by either PCR or restriction enzyme digestion. A selection of different size clones for each targeted sequence are chosen for transposon—hopping and DNA sequencing.

Success of the method, like any cDNA cloning method, depends on the quality of the libraries employed. High complexity and large average insert size are required. We have employed HPLC as a means of fractionating cDNA for the purpose of constructing libraries.

A. Construction of Size Fractionated cDNA Libraries

PolyA+ RNA is purchased from Clontech, treated with DNase I to remove traces of genomic DNA contamination and converted into double stranded cDNA using the SuperScript™ Plasmid System for cDNA Synthesis and Plasmid Cloning (Life Technologies). No radioisotope is incorporated in either of the cDNA synthesis steps. The cDNA is then size fractionated on a TransGenomics HPLC system equipped with a size exclusion column (TosoHass) with dimensions of 7.8 mm×30 cm and a particle size of 10 μm. Tris buffered saline (TBS) is used as the mobile phase, and the column is run at a flow rate of 0.5 mL/min. The system is calibrated by running a 1 kb ladder through the column and analyzing the fractions by agarose gel electrophoresis. Using these data, it could be determined which fractions are to be pooled to obtain the largest cDNA library. Generally, fractions that elute in the range of 12 to 15 minutes are used.

The cDNA is precipitated, concentrated and then ligated into the SalI/NotI sites in pSPORT. After electroporation into E. coli strain DH12S, colonies are subjected to a miniprep procedure and the resulting cDNA is digested using SalI/NotI restriction enzymes. Generally, the average insert size of libraries made in this fashion was greater the 3.5 Kb; the overall complexity of the library is optimally greater than 10⁷ independent clones. The library is amplified in semi-solid agar for 2 days at 30° C. An aliquot (200 microliters) of the amplified library is inoculated into a 200 mL culture for single-stranded DNA isolation by super-infection with an f1 helper phage. The single stranded circular DNA is concentrated by ethanol precipitation, resuspended at a concentration of one microgram per microliter and used for the cDNA capture experiments.

B. Conversion of Double-Stranded cDNA Libraries into Single-Stranded Circular Form

To prepare cultures, 200 mL LB with 400 μL carbenicillin (100 mg/mL stock solution) are inoculated with from 200 μL to 1 mL of thawed cDNA library and incubated at 37° C. while shaking at 250 rpm for approximately 45 minutes, or until an OD600 of 0.025-0.040 is attained. M13K07 helper phage (1 mL) is added to the culture and grown for 2 hours, after which Kanamycin (500 μl; 30 mg/mL) is added and the culture is grown for an additional 15-18 hours.

The culture is then poured into 6 screw-cap tubes (50 mL autoclaved tubes) and cells subjected to centrifugation at 10K in an HB-6 rotor for 15 minutes at 4° C. to pellet the cells. The supernatant is filtered through a 0.2 μm filter and 12,000 units of Gibco DNase I was added. The mixture is incubated for 90 minutes at room temperature.

For PEG precipitation, 50 mL of ice-cold 40% PEG 8000, 2.5 M NaCl, and 10 mM MgSO₄ are added to the supernatant, mixed, and aliquotted into 6 centrifuge tubes (covered with parafilm). The tubes and contents are incubated for 1 hour on wet ice or at 4° C. overnight. The tubes are then centrifuged at 10K in a HB-6 rotor for 20 minutes at 4° C. to pellet the helper phage.

Following centrifugation, the supernatant is discarded and the sides of the tubes are dried. Each pellet is resuspended in 1 mL TE, pH 8. The resuspended pellets are pooled into a 14 mL tube (Sarstadt), (6 mL total). SDS is added to 0.1% (60 μl of stock 10% SDS). Freshly made proteinase K (20 mg/mL) is added (60 μl) and the suspension is incubated for 1 hour at 42° C.

For phenol/chloroform extractions, 1 mL of NaCl (5M) is added to the suspension in the tube. An equal volume of phenol/chloroform (6 mL) is added and the contents are vortexed or shaken. The suspension is then centrifuged at 5K in an HB-6 rotor for 5 minutes at 4° C. The aqueous (top) phase is transferred to a new tube (Sarstadt) and extractions are repeated until no interface is visible.

Ethanol precipitation is then performed on the aqueous phase whose volume is divided into 2 tubes (3 mL each). To each tube, 2 volumes of 100% ethanol are added and precipitation is carried out overnight at −20° C. The precipitated DNA is pelleted at 10K in an HB-6 rotor for 20 minutes at 4° C. The ethanol is discarded. Each pellet is resuspended in 700 μl of 70% ethanol. The contents of each tube are combined into one micro centrifuge tube and centrifuged in a micro centrifuge (Eppendorf) at 14K for 10 minutes at 4° C. After discarding the ethanol, the DNA pellet is dried in a speed vacuum. In order to remove oligosaccharides, the pellet is resuspended in 50 μl TE buffer, pH 8. The resuspension is incubated on dry ice for 10 minutes and centrifuged at 14K in an Eppendorf microfuge for 15 minutes at 4° C. The supernatant is then transferred to a new tube and the final volume is recorded.

To check purity, DNA is diluted 1:100 and added to a micro quartz cuvette, where DNA is analyzed by spectrometry at an OD260/OD280. The preferred purity ratio is between 1.7 and 2.0. The DNA is diluted to 1 μg/μL in TE, pH 8 and stored at 4° C. The concentration of DNA is calculated using the formula: (32 μg/mL*OD)(mL/1000 μL)(100)(OD260). The quality of single-stranded DNA is determined by first mixing 1 μL of 5 ng/μl ssDNA; 11 μL deionized water; 1.5 μL 10 μM T7 sport primer (fresh dilution of stock); 1.5 μl 10× Precision-Taq buffer per reaction. In the repair mix, a cocktail of 4 μl of 5 mM dNTPs (1.25 mM each); 1.5 μL 10× Precision-Taq buffer; 9.25 μL deionized water; and 0.25 μL Precision-Taq polymerase is mixed per reaction and preheated at 70° C. until the middle of the thermal cycle.

The DNA mixes are aliquotted into PCR tubes and the thermal cycle is started. The PCR thermal cycle consists of 1 cycle at 95° C. for 20 sec.; 59° C. for 1 min. (15 μL repair mix added); and 73° C. for 23 minutes. For ethanol precipitation, 15 μg glycogen, 16 μl ammonium acetate (7.5M), and 125 μL 100% ethanol are added and the contents are centrifuged at 14K in an Eppendorf microfuge for 30 minutes at 4° C. The resulting pellet is washed 1 time with 125 μL 70% ethanol and then the ethanol is discarded. The pellet is dried in a speed vacuum and resuspended in 10 μL TE buffer, pH 8.

Single-stranded DNA is electroporated into E. coli DH10B or DH12S cells by pre-chilling the cuvettes and sliding holder, and thawing the cells on ice-water. DNA is aliquotted into micro centrifuge tubes (Eppendorf) as follows: 2 μL repaired library, (=1×10⁻³ μg); 1 μL unrepaired library (1 ng/μL), (=1×10⁻³ μg); and 1 μL pUC19 positive control DNA (0.01 μg/μL), (=1×10⁻⁵ μg). The mixtures are stored on ice until use.

One at a time, 40 μL of cells are added to a DNA aliquot. The cell/DNA mixture is not pipetted up and down more than one time. The mixture is then transferred via pipette into a cuvette between the metal plates, and electroporation is performed at 1.8 kV. Immediately afterward, 1 mL SOC medium (i.e., SOB (bacto-tryptone; bacto-yeast extract; NaCl)+glucose (20 mM)+Mg²⁺) (See, J. Sambrook et al., Molecular Cloning: A Laboratory Manual, 2nd Ed., A.2, 1989) is added to the cuvette and the contents are transferred into 15 mL of media, as commonly known in the art. The cells are allowed to recover for 1 hour at 37° C. with shaking (225 rpm).

Serial dilutions of the culture are made in 1:10 increments (20 μL into 180 μL LB) for plating the electroporated cells. For the repaired library, dilutions of 1:100, 1:1000, 1:10,000 are made. For the unrepaired library, dilutions of 1:10 and 1:100 are made. Positive control dilutions of 1:10 and 1:100 are made. Each dilution (100 μL) is plated onto small plates containing LB+carbenicillin and incubated at 37° C. overnight. The titer and background are calculated by methods well known in the art. Specifically, the colonies on each plate are counted using the lowest dilution countable. The titer is calculated using the formula: (# of colonies)(dilution factor)(200 μL/100 μL)(1000 μL/20 μL)=CFUs, where CFUs/μg DNA used=CFU/μg. The % background=((unrepaired CFU/μg)/(repaired CFU/μg))×100%.

C. Solution Hybridization and DNA Capture

One microliter of anti-sense biotinylated oligonucleotides (or sense oligonucleotides when annealing to single-stranded DNA from pSPORT2 vector) containing 150 ng of up to 50 different 80-mer oligonucleotide probes is added to 6 μL (6 μg) of a mixture of up to 15 single-stranded, covalently-closed, circular cDNA libraries and 7 μL of 100% formamide in a 0.5 mL PCR tube. Examples of sequences of 80mer oligos that may be used in the present invention are: 5′TTGGTGGTCAGAGACGGGTCATCTGTCGCTAAGGCGCAACCTCCAGGGAA CTCGAGGCCTGCCAGGGTCTGTCCAGATCA-3′ (SEQ ID NO:7) for BMSOTR, and 5′TGAGAGAGTGACACTGAAGTTGTCCCCTTCCTCCACTCTCCTATTCCCTTC TCATGTTTACATTTCCCTATGCTCTTCCA-3′ (SEQ ID NO:10) for the BMSOTR variant.

The mixture is heated in a thermal cycler to 95° C. for 2 minutes. Fourteen microliters of 2× hybridization buffer (50% formamide, 1.5 M NaCl, 0.04 M NaPO₄, pH 7.2, 5 mM EDTA, 0.2% SDS) are added to the heated probe/cDNA library mixture and incubated at 42° C. for 26 hours. Hybrids between the biotinylated oligo and the circular cDNA are isolated by diluting the hybridization mixture to 220 microliters in a solution containing 1 M NaCl, 10 mM Tris-HCl pH 7.5, 1 mM EDTA, pH 8.0 and adding 125 microliters of streptavidin magnetic beads. This solution is incubated at 42° C. for 60 minutes, and mixed every 5 minutes to resuspend the beads. The beads are separated from the solution with a magnet and washed three times in 200 microliters of 0.1×SSPE, 0.1% SDS at 45° C.

The single stranded cDNAs are released from the biotinylated oligo/streptavidin magnetic bead complex by adding 50 microliters of 0.1 N NaOH and incubating at room temperature for 10 minutes. Six microliters of 3 M sodium acetate are added along with 15 μg of glycogen and the solution is ethanol precipitated with 120 microliters of 100% ethanol. The precipitated DNA is resuspend in 12 μL of TE (10 mM Tris-HCl, pH 8.0, 1 mM EDTA, pH 8.0). The single stranded cDNA is converted into double strands in a thermal cycler by mixing 5 μL of the captured DNA with 1.5 μL of 10 μM of standard SP6 Sport primer: 5′-ATTTAGGTGACACTATAG-3′(SEQ ID NO:11) for libraries in pSPORT 1 and 2, and T7 Sport primer: 5′-TAATACGACTCACTATAGGG-3′ (SEQ ID NO:12) for libraries in pCMVSPORT, and 1.5 μL of 10×PCR buffer. The mixture is heated to 95° C. for 20 seconds, and then ramped down to 59° C. At this time 15 μL of a repair mix, preheated to 70° C., is added to the DNA (Repair mix contains 4 μL of 5 mM dNTPs (1.25 mM each), 1.5 μL of 10×PCR buffer, 9.25 μL of water, and 0.25 μL of Taq polymerase). The solution is ramped back to 73° C. and incubated for 23 minutes.

The repaired DNA is ethanol precipitated and resuspended in 10 μL of TE. Two μL are electroporated per tube containing 40 μL of E. coli DH12S cells. Three hundred and thirty three μL (333 μL) are plated onto one 150 mm plate of LB agar plus 100 μg/mL of ampicillin. After overnight incubation at 37° C., the colonies from all plates are harvested by scraping into 10 mL of LB+50 μg/mL of ampicillin and 2 mL of sterile glycerol.

The second round of selection is initiated by making single-strand circular DNA from the primary selected library using the above-described method. The purified single-stranded circular DNA is then assayed with gene-specific primers(GSPs) (for example, for BMSOTR: left primer 1: 5′TGCGAAAGTGCCTTCTAGGT-3′ (SEQ ID NO:5), right primer 1: 5′CAGTGGCTCACTGTCTCCAA-3′ (SEQ ID NO:6); for the BMSOTR variant: left primer 1: 5′-TGCGAAAGTGCCTTCTAGGT-3′ (SEQ ID NO:8), right primer 2: 5′CAGTGGCTCACTGTCTCCAA-3′ (SEQ ID NO:9)), for each of the targeted sequences using standard PCR conditions. The hybridization is performed including only those 80 mer biotinylated probes (for example, SEQ ID NOS:7 and 10) whose targeted sequences have a positive result with the GSPs. The resulting single-stranded circular DNA is converted into double strands using the antisense oligo for each target sequence as the repair primer (the sense primer was used for material captured from pSPORT2 libraries). The resulting double stranded DNA is electroporated into DHIOB cells and the resulting colonies are inoculated into 96 deep well blocks. After overnight growth, DNA is prepared and sequentially screened for each of the targeted sequences using the GSPs. The DNA is also digested with SalI and NotI restriction enzymes and the inserts are sized by agarose gel electrophoresis.

### Example 4

### RNA Ligase Protocol for Generating the 5′ OR 3′ End Sequences to Obtain the Full-Length BMSOTR Gene

Once a BMSOTR gene/polynucleotide sequence of interest is identified, several methods are available for the identification of 5′ or 3′ portions of the gene which may not be present in the original cDNA plasmid. These methods include, but are not limited to, filter probing, clone enrichment using specific probes and protocols similar and identical to 5′ and 3′RACE. While the full-length gene may be present in the library and can be identified by probing, a useful method for generating the 5′ or 3′ end is to use the existing sequence information from the original cDNA to generate the missing information. A method similar to 5′RACE is available for generating the missing 5′ end of a desired full-length gene. (This method was published by Fromont-Racine et al., Nucleic Acids Res., 21(7): 1683-1684 (1993)).

Briefly, a specific RNA oligonucleotide is ligated to the 5′ ends of a population of RNA, preferably 30, containing full-length gene RNA transcripts and a primer set containing a primer specific to the ligated RNA oligonucleotide and a primer specific to a known sequence of the gene of interest, and is used to PCR amplify the 5′ portion of the desired full length gene which may then be sequenced and used to generate the full-length gene. This method starts with total RNA isolated from the desired source. PolyA RNA may be used, but is not a prerequisite for this procedure. The RNA preparation is then be treated with phosphatase if necessary to eliminate 5′ phosphate groups on degraded or damaged RNA which may interfere with the later RNA ligase step. The phosphatase, if used, is then inactivated and the RNA is treated with tobacco acid pyrophosphatase in order to remove the cap structure present at the 5′ ends of messenger RNAs. This reaction leaves a 5′ phosphate group at the 5′ end of the cap cleaved RNA which can then be ligated to an RNA oligonucleotide using T4 RNA ligase. This modified RNA preparation can then be used as a template for first strand cDNA synthesis using a gene specific oligonucleotide. The first strand synthesis reaction can then be used as a template for PCR amplification of the desired 5′ end using a primer specific to the ligated RNA oligonucleotide and a primer specific to the known sequence of the apoptosis related of interest. The resultant product is then sequenced and analyzed to confirm that the 5′ end sequence belongs to the relevant sequence of interest.

### Example 5

Signal Transduction Assays

The activity of BMSOTR or homologues thereof, can be measured using any assay suitable for the measurement of the activity of a G protein-coupled receptor, as commonly known in the art. Signal transduction activity of a G protein-coupled receptor can be determined by monitoring intracellular Ca²⁺, cAMP, inositol-1,4,5-triphosphate (IP₃), or 1,2-diacylglycerol (DAG). Assays for the measurement of intracellular Ca²⁺ are described, for example, in Sakurai et al. (EP 480 381). Intracellular IP₃ can be measured using a kit available from Amersham, Inc. (Arlington Heights, Ill.). A kit for measuring intracellular cAMP is available from Diagnostic Products, Inc. (Los Angeles, Calif.).

Activation of a G protein-coupled receptor triggers the release of Ca²⁺ ions sequestered in the mitochondria, endoplasmic reticulum, and other cytoplasmic vesicles into the cytoplasm. Fluorescent dyes, e.g., fura-2, can be used to measure the concentration of free cytoplasmic Ca²⁺. The ester of fura-2, which is lipophilic and can diffuse across the cell membrane, is added to the culture medium of the host cells which recombinantly express BMSOTR. Once inside the cell, the fura-2 ester is hydrolyzed by cytosolic esterases to its non-lipophilic form, and then the dye cannot diffuse out of the cell. The non-lipophilic form of fura-2 fluoresces when it binds to free Ca 2+. The fluorescence can be measured without lysing the cells at an excitation spectrum of 340 nm or 380 nm and at fluorescence spectrum of 500 nm (Sakurai et al., EP 480 381).

Upon activation of BMSOTR, the rise of free cytosolic Ca²⁺ concentration is preceded by the hydrolysis of phosphatidylinositol 4,5-bisphosphate. Hydrolysis of this phospholipid by the phospholipase, phospholipase C, yields 1,2-diacylglycerol (DAG), which remains in the membrane, and water-soluble inositol 1,4,5-triphosphate (IP₃). Binding of ligands or agonists will increase the concentration of DAG and IP₃. Thus, signal transduction activity can be measured by monitoring the concentration of these hydrolysis products.

To measure IP₃ concentration, radioactively-labeled ([³H])-inositol is added to the culture medium of host cells expressing BMSOTR. The ³H-inositol is taken up by the cells and incorporated into IP₃. The resulting inositol triphosphate is separated from the mono- and di-phosphate forms and measured (Sakurai et al., EP 480 381). Alternatively, an inositol 1,4,5-triphosphate assay system (Amersham) is commercially available for such use. With this system, the supplier (Amersham) provides tritium-labeled inositol 1,4,5-triphosphate and a receptor capable of distinguishing the radioactive inositol from other inositol phosphates. With these reagents, an effective and accurate competition assay can be performed to determine the inositol triphosphate levels.

Cyclic AMP levels can be measured according to the methods described in Gilman et al., Proc. Natl. Acad. Sci, 67:305-312 (1970). In addition, a kit for assaying levels of cAMP is available from Diagnostic Products Corp. (Los Angeles, Calif.).

### Example 6

### Expression Profiling of Novel Human GPCR BMSOTR Polypeptide

The same PCR primer pairs that are used to identify BMSOTR cDNA clones are used to measure the steady state levels of mRNA by quantitative PCR. For example, the PCR primer pairs SEQ ID NOS:5-6 are used to measure the steady state levels of the newly described BMSOTR mRNA by quantitative PCR.

Briefly, first strand cDNA is made from commercially available mRNA (Clontech) and subjected to real time quantitative PCR using a PE 5700 instrument (Applied Biosystems, Foster City, Calif.) which detects the amount of DNA amplified during each cycle by the fluorescent output of SYBR green, a DNA binding dye specific for double strands. The specificity of the primer pair for its target is verified by performing a thermal denaturation profile at the end of the run which provides an indication of the number of different DNA sequences present by determining melting Tm. The contribution of contaminating genomic DNA to the assessment of tissue abundance is controlled for by performing the PCR with first strand cDNA made with and without reverse transcriptase. In all cases, the contribution of material amplified in the no reverse transcriptase controls is negligible.

Small variations in the amount of cDNA in each tube are determined by performing a parallel experiment using a primer pair for the cyclophilin gene, which is expressed in equal amounts in all tissues. These data are used to normalize the data obtained with the primer pairs. The PCR data is converted into a relative assessment of the differences in transcript abundance among the tissues tested and the data is presented in FIG. 6. Transcripts corresponding to the orphan GPCR, BMSOTR, were expressed at high levels in the pancreas and brain; significantly in the testis, and to a lesser extent in other tissues as shown.

### Example 7

### Method of Assessing the Expression Profile of the Novel BMSOTR Polypeptides of the Present Invention Using Expanded mRNA Tissue and Cell Sources

Total RNA from tissues was isolated using the TriZol protocol (Invitrogen) and quantified by determining its absorbance at 260 nM. An assessment of the 18s and 28s ribosomal RNA bands was made by denaturing gel electrophoresis to determine RNA integrity.

The specific sequence to be measured was aligned with related genes found in GenBank to identity regions of significant sequence divergence to maximize primer and probe specificity. Gene-specific primers and probes were designed using the ABI primer express software to amplify small amplicons (150 base pairs or less) to maximize the likelihood that the primers function at 100% efficiency. All primer/probe sequences were searched against Public Genbank databases to ensure target specificity. Primers and probes were obtained from ABI.

For BMSOTR, the primer probe sequences were as follows

| | |
|---|---|
| Forward Primer | |
| 5′-CCTTGCTCCTGCGAAAGTG-3′ | (SEQ ID NO:53) |
| Reverse Primer | |
| 5′-GCGCCTTAGCGACAGATGA-3′ | (SEQ ID NO:54) |
| TaqMan Probe | |
| 5′-CCGTCTCTGACCACCAAGCACCTAGAAG-3′ | (SEQ ID NO:55) |

I. DNA Contamination

To access the level of contaminating genomic DNA in the RNA, the RNA was divided into 2 aliquots and one half was treated with Rnase-free Dnase (Invitrogen). Samples from both the Dnase-treated and non-treated were then subjected to reverse transcription reactions with (RT+) and without (RT−) the presence of reverse transcriptase. TaqMan assays were carried out with gene-specific primers (see above) and the contribution of genomic DNA to the signal detected was evaluated by comparing the threshold cycles obtained with the RT+/RT− non-Dnase treated RNA to that on the RT+/RT− Dnase treated RNA. The amount of signal contributed by genomic DNA in the Dnased RT− RNA must be less that 10% of that obtained with Dnased RT+RNA. If not the RNA was not used in actual experiments.

II. Reverse Transcription Reaction and Sequence Detection

100 ng of Dnase-treated total RNA was annealed to 2.5 μM of the respective gene-specific reverse primer in the presence of 5.5 mM Magnesium Chloride by heating the sample to 72° C. for 2 min and then cooling to 55° C. for 30 min. 1.25 U/μl of MuLv reverse transcriptase and 500 μM of each dNTP was added to the reaction and the tube was incubated at 37° C. for 30 min. The sample was then heated to 90° C. for 5 min to denature enzyme.

Quantitative sequence detection was carried out on an ABI PRISM 7700 by adding to the reverse transcribed reaction 2.5 μM forward and reverse primers, 2.0 μM of the TaqMan probe, 500 μM of each dNTP, buffer and 5U AmpliTaq Gold™. The PCR reaction was then held at 94° C. for 12 min, followed by 40 cycles of 94° C. for 15 sec and 60° C. for 30 sec.

III. Data Handling

The threshold cycle (Ct) of the lowest expressing tissue (the highest Ct value) was used as the baseline of expression and all other tissues were expressed as the relative abundance to that tissue by calculating the difference in Ct value between the baseline and the other tissues and using it as the exponent in 2^{(ΔCt)}

The expanded expression profile of the BMSOTR polypeptide is provided in FIG. 7 and described elsewhere herein.

### Example 7

### BMSOTR Activity

This example describes another method for screening compounds which are BMSOTR antagonists, and thus inhibit the activation or function of the BMSOTR polypeptide of the present invention. The method involves determining inhibition of binding of a labeled ligand, such as dATP, dAMP, or UTP, to cells expressing the novel BMSOTR on the cell surface, or to cell membranes containing the BMSOTR.

Such a method further involves transfecting a eukaryotic cell with DNA encoding a BMSOTR polypeptide such that the cell expresses the receptor on its surface. The cell is then contacted with a potential antagonist in the presence of a labeled form of a ligand, such as dATP, dAMP, or UTP. The ligand can be labeled, e.g., by radioactivity, fluorescence, chemiluminescence, or any other suitable detectable label commonly known in the art. The amount of labeled ligand bound to the expressed BMSOTR receptors is measured, e.g., by measuring radioactivity associated with transfected cells, or membranes from these cells. If the compound binds to the expressed BMSOTR, the binding of labeled ligand to the receptor is inhibited, as determined by a reduction of labeled ligand which also binds to the BMSOTR. This method is called a binding assay. The above-described technique can also be used to determine binding of BMSOTR agonists.

In a further screening procedure, mammalian cells, for example, but not limited to, CHO, HEK 293, Xenopus oocytes, RBL-2H3, etc., which are transfected with nucleic acid encoding a novel BMSOTR, are used to express the receptor of interest. The cells are loaded with an indicator dye that produces a fluorescent signal when bound to calcium, and the cells are contacted with a test substance and a receptor agonist, such as DATP, DAMP, or UTP. Any change in fluorescent signal is measured over a defined period of time using, for example, a fluorescence spectrophotometer or a fluorescence imaging plate reader. A change in the fluorescence signal pattern generated by the ligand relative to control indicates that a compound is a potential antagonist or agonist for the receptor.

In yet another screening procedure, mammalian cells are transfected with a BMSOTR-encoding polynucleotide sequence so as to express the BMSOTR of interest. The same cells are also transfected with a reporter gene construct that is coupled to/associated with activation of the receptor. Nonlimiting examples of suitable reporter gene systems include luciferase or beta-galactosidase regulated by an appropriate promoter. The engineered cells are contacted with a test substance or compound and a receptor ligand, such as dATP, dAMP, or UTP, and the signal produced by the reporter gene is measured after a defined period of time. The signal can be measured using a luminometer, spectrophotometer, fluorimeter, or other such instrument appropriate for the specific reporter construct used. Inhibition of the signal generated by the ligand indicates that a compound is a potential antagonist for the receptor.

Another screening technique for determining BMSOTR antagonists or agonists involves introducing RNA encoding the BMSOTR polypeptide into cells (e.g., CHO, HEK 293, RBL-2H3 cells, and the like) in which the receptor is transiently or stably expressed. The receptor cells are then contacted with a ligand for the BMSOTR, such as dATP, dAMP, or UTP, and a compound to be screened. Inhibition or activation of the receptor is then determined by detection of a signal, such as, cAMP, calcium, proton, or other ions.

### Example 8

### Functional Characterization of the Novel Human GPCR, BMSOTR

The use of mammalian cell reporter assays to demonstrate functional coupling of known GPCRs has been well documented in the literature (Gilman, 1987 Ann. Rev. Biochem. 56: 615-649; Boss et al., 1996, J. Biol. Chem . . . , 271: 10429-14032; Alam & Cook, 1990, Anal. Biochem., 188: 245-254; George et al., 1997, J. Neurochem., 69: 1278-1285; Selbie & Hill, 1998, TiPs, 19: 87-93; Rees et al., 1999, In Milligan G. (ed.): Signal Transduction: A practical approach, Oxford: Oxford Univ. Press, 171-221). In fact, reporter assays have been successfully used for identifying novel small molecule agonists or antagonists against GPCRs as a class of drug targets (Zlokarnik et al., 1998, Science, 279: 84-88; George et al; Boss et al.; Rees et al.). In such reporter assays, a promoter is regulated as a direct consequence of activation of specific signal transduction cascades following agonist binding to a GPCR (Alam & Cook 1990; Selbie & Hill, 1998; Boss et al.; George et al. 1997; Gilman, 1987).

A number of response element-based reporter systems have been developed that enable the study of GPCR function. These include cAMP response element (CRE)-based reporter genes for G alpha i/o, G alpha s-coupled GPCRs, Nuclear Factor Activator of Transcription (NFAT)-based reporters for G alpha q/11—coupled receptors and MAP kinase reporter genes for use in G alpha i/o coupled receptors (Selbie & Hill, 1998; Boss et al. 1996; George et al. 1997; Gilman 1987; Rees et al. 1999). Transcriptional response elements that regulate the expression of Beta-Lactamase within a CHO K1 cell line (Cho/NFAT-CRE: Aurora Biosciences™) (Zlokarnik et al., 1998) have been implemented to characterize the function of the BMSOTR polypeptide of the present invention. The system enables demonstration of constitutive G-protein coupling to endogenous cellular signaling components upon intracellular overexpression of GPCR receptors. Overexpression has been shown to represent a physiologically relevant event. For example, it has been shown that overexpression occurs in nature during metastatic carcinomas, wherein defective expression of the monocyte chemotactic protein 1 receptor, CCR2, in macrophages is associated with the incidence of human ovarian carcinoma (Sica, et al., 2000, J. Immunol., 164: 733-8; Salcedo et al., 2000, Blood, 96(1): 34-40). Indeed, it has been shown that overproduction of the Beta 2 Adrenergic Receptor in transgenic mice leads to constitutive activation of the receptor signaling pathway such that these mice exhibit increased cardiac output (Kypson et al., 1999, Gene Therapy, 6: 1298-1304; Dorn et al., 1999, PNAS, 96: 6400-5). These are only a few of the many examples demonstrating constitutive activation of GPCRs whereby many of these receptors are likely to be in the active, R*, conformation (Wess, 1997, FASEB J., 11(5): 346-354).

A. Materials and Methods

### DNA Constructs

The putative GPCR BMSOTR cDNA can be PCR amplified using PFU™ (Stratagene) and gene specific primers such as SEQ I) NOS:5-6. A 3 prime or 5 prime primer can be used to add a Flag-tag epitope to the BMSOTR polypeptide for immunocytochemistry. The product from the PCR reaction is isolated from a 0.8% Agarose gel (Invitrogen) and purified using a Gel Extraction Kit™ from Qiagen.

The purified product is digested overnight along with the pcDNA3.1 Hygro™ mammalian expression vector from Invitrogen using the HindIII and BamHI restriction enzymes (New England Biolabs). These digested products are then purified using the Gel Extraction Kit™ from Qiagen and subsequently ligated to the pcDNA3.1 Hygro™ expression vector using a DNA molar ratio of 4 parts insert: 1 vector. All DNA modification enzymes are purchased from NEB. The ligation may be incubated overnight at 16 degrees Celsius, after which time, one microliter of the mix may be used to transform DH5 alpha cloning efficiency competent E. coli™ (Gibco BRL). A detailed description of the pcDNA3.1 Hygro™ mammalian expression vector is available from Invitrogen (1600 Faraday Avenue, P.O. Box 6482, Carlsbad, Calif. 92008). The plasmid DNA from the ampicillin resistant clones are isolated using the Wizard DNA Miniprep System™ from Promega. Positive clones are then confirmed and scaled up for purification using the Qiagen Maxiprep™ plasmid DNA purification kit.

B. Cell Line Generation

The pcDNA3.1hygro vector containing the GPCR BMSOTR cDNA are used to transfect Cho/NFAT-CRE (Aurora Biosciences) cells using Lipofectamine 2000™ according to the manufacturers specifications (Gibco BRL). Two days later, the cells are split 1:3 into selective media (DMEM 11056, 600 ug/ml Hygromycin, 200 ug/ml Zeocin, 10% FBS). All cell culture reagents are purchased from Gibco BRL-Invitrogen.

The Cho/NFAT-CRE cell lines, transiently or stably transfected with the BMSOTR GPCR, are analyzed using the FACS Vantage SE™ (BD), fluorescence microscopy (Nikon), and the UL Analyst™ (Molecular Devices). In this system, changes in real-time gene expression, as a consequence of constitutive G-protein coupling of the BMSOTR GPCR, is examined by analyzing the fluorescence emission of the transformed cells at 447 nm and 518 nm. The changes in gene expression can be visualized using Beta-Lactamase as a reporter, that, when induced by the appropriate signaling cascade, hydrolyzes an intracellularly loaded, membrane-permeant ester, Cephalosporin-Coumarin-Fluorescein-2/Acetoxymethyl™ (CCF2/AM™ Aurora Biosciences; Zlokarnik, et al., 1998). The CCF2/AM™ substrate is a 7-hydroxycoumarin cephalosporin with a fluorescein attached through a stable thioether linkage. Induced expression of the Beta-Lactamase enzyme is readily apparent since each enzyme molecule produced is capable of changing the fluorescence of many CCF2/AM™ substrate molecules. A schematic is provided below.

[image]

In summary, CCF2/AM™ is a membrane permeant, intracellularly-trapped, fluorescent substrate with a cephalosporin core that links a 7-hydroxycoumarin to a fluorescein. For the intact molecule, excitation of the coumarin at 409 nm results in Fluorescence Resonance Energy Transfer (FRET) to the fluorescein which emits green light at 518 nm. Production of active Beta-Lactamase results in cleavage of the Beta-Lactam ring, leading to disruption of FRET, and excitation of the coumarin only—thus giving rise to blue fluorescent emission at 447 nm.

Fluorescent emissions are detected using a Nikon-TE300 microscope equipped with an excitation filter (D405/10X-25), dichroic reflector (430DCLP), and a barrier filter for dual DAPI/FITC (510 nM) to visually capture changes in Beta-Lactamase expression. The FACS Vantage SE is equipped with a Coherent Enterprise II Argon Laser and a Coherent 302C Krypton laser. In flow cytometry, UV excitation at 351364 nm from the Argon Laser or violet excitation at 407 nm from the Krypton laser are used. The optical filters on the FACS Vantage SE are HQ460/50m and HQ535/40m bandpass separated by a 490 dichroic mirror.

Prior to analyzing the fluorescent emissions from the cell lines as described above, the cells are loaded with the CCF2/AM substrate. A 6× CCF2/AM loading buffer may be prepared whereby 1 mM CCF2/AM (Aurora Biosciences) may be dissolved in 100% DMSO (Sigma). 12 ul of this stock solution may be added to 60 ul of 100 mg/ml Pluronic F127 (Sigma) in DMSO containing 0.1% Acetic Acid (Sigma). This solution may be added while vortexing to 1 mL of Sort Buffer (PBS minus calcium and magnesium-Gibco-25 mM HEPES-Gibco-pH 7.4, 0.1% BSA). Cells are placed in serum-free media and the 6×CCF2/AM may be added to a final concentration of 1×. The cells are then loaded at room temperature for one to two hours, and then subjected to fluorescent emission analysis as described herein. Additional details relative to the cell loading methods and/or instrument settings may be found by reference to the following publications: see Zlokarnik, et al., 1998; Whitney et al., 1998, Nature Biotech. 16: 1329-1333; and BD Biosciences, 1999, FACS Vantage SE Training Manual.

C. Immunocytochemistry

The cell lines transfected and selected for expression of Flag-epitope tagged GPCRs are analyzed by immunocytochemistry. The cells are plated at 1×10\hat{ }3 in each well of a glass slide (VWR). The cells are rinsed with PBS followed by acid fixation for 30 minutes at room temperature using a mixture of 5% Glacial Acetic Acid/90% ETOH. The cells are then blocked in 2% BSA and 0.1% Triton in PBS, incubated for 2 h at room temperature or overnight at 4° C. A monoclonal anti-Flag FITC antibody may be diluted at 1:50 in blocking solution and incubated with the cells for 2 h at room temperature. Cells are then washed three times with 0.1% Triton in PBS for five minutes. The slides are overlayed with mounting media dropwise with Biomedia—Gel Mount™ (Biomedia; Containing Anti-Quenching Agent). Cells are examined at 10× magnification using the Nikon TE300 equiped with FITC filter (535 nm).

D. Demonstration of Cell Surface Expression

BMSOTR may be tagged at the C-terminus using the Flag epitope and inserted into the pcDNA3.1 hygro™ expression vector, as described herein. Immunocytochemistry of Cho Nfat-CRE cell lines transfected with the Flag-tagged BMSOTR construct with FITC conjugated Anti Flag monoclonal antibody demonstrated that BMSOTR is indeed a cell surface receptor. The immunocytochemistry also confirmed expression of the BMSOTR in the Cho Nfat-CRE cell lines. Briefly, Cho Nfat-CRE cell lines are transfected with pcDNA3.1 hygro™/BMSOTR-Flag vector, fixed with 70% methanol, and permeablized with 0.1% TritonX100. The cells are then blocked with 1% Serum and incubated with a FITC conjugated Anti Flag monoclonal antibody at 1:50 dilution in PBS-Triton. The cells are then washed several times with PBS-Triton, overlayed with mounting solution, and fluorescent images are captured. The control cell line, non-transfected ChoNfat CRE cell line, exhibited no detectable background fluorescence. Plasma membrane localization would be consistent with BMSOTR representing a 7 transmembrane domain containing GPCR.

E. Screening Paradigm

The Aurora Beta-Lactamase technology provides a clear path for identifying agonists and antagonists of the BMSOTR polypeptide. Cell lines that exhibit a range of constitutive coupling activity may be identified by sorting through BMSOTR transfected cell lines using the FACS Vantage SE (see supra). For example, cell lines that exhibit an intermediate coupling response, using the LJL analyst, would provide the opportunity to screen, indirectly, for both agonists and antogonists of BMSOTR by looking for inhibitors that block the beta lactamase response, or agonists that increase the beta lactamase response. As described herein, modulating the expression level of beta lactamase directly correlates with the level of cleaved CCR2 substrate. For example, this screening paradigm has been shown to work for the identification of modulators of a known GPCR, 5HT6, that couples through Adenylate Cyclase, in addition to, the identification of modulators of the 5HT2c GPCR, that couples through changes in [Ca²⁺]i. BMSOTR modulator screens may be carried out using a variety of high throughput methods known in the art, though preferably using the fully automated Aurora UHTSS system.

In preferred embodiments, the BMSOTR transfected Cho Nfat-CRE cell lines are useful for the identification of agonists and antagonists of the BMSOTR polypeptide. Representative uses of these cell lines would be their inclusion in a method of identifying BMSOTR agonists and antagonists. Preferably, the cell lines are useful in a method for identifying a compound that modulates the biological activity of the BMSOTR polypeptide, comprising the steps of (a) combining a candidate modulator compound with a host cell expressing the BMSOTR polypeptide having the sequence as set forth in SEQ ID NO:2; and (b) measuring an effect of the candidate modulator compound on the activity of the expressed BMSOTR polypeptide. Representative vectors expressing the BMSOTR polypeptide are referenced herein (e.g., pcDNA3.1 hygro™) or otherwise known in the art.

The cell lines are also useful in a method of screening for a compound that is capable of modulating the biological activity of BMSOTR polypeptide, comprising the steps of: (a) determining the biological activity of the BMSOTR polypeptide in the absence of a modulator compound; (b) contacting a host cell expression the BMSOTR polypeptide with the modulator compound; and (c) determining the biological activity of the BMSOTR polypeptide in the presence of the modulator compound; wherein a difference between the activity of the BMSOTR polypeptide in the presence of the modulator compound and in the absence of the modulator compound indicates a modulating effect of the compound. Additional uses for these cell lines are described herein or otherwise known in the art.

### Example 9

### Method of Creating N- and C-Terminal Deletion Mutants Corresponding to the BMSOTR Polypeptide of the Present Invention

As described elsewhere herein, the present invention encompasses the creation of N- and C-terminal deletion mutants, in addition to any combination of N- and C-terminal deletions thereof, corresponding to the BMSOTR polypeptide of the present invention. A number of methods are available to one skilled in the art for creating such mutants. Such methods may include a combination of PCR amplification and gene cloning methodology. Although one of skill in the art of molecular biology, through the use of the teachings provided or referenced herein, and/or otherwise known in the art as standard methods, could readily create each deletion mutant of the present invention, exemplary methods are described below.

Briefly, using the isolated cDNA clone encoding the full-length BMSOTR polypeptide sequence (as described in Example 3, for example), appropriate primers of about 15-25 nucleotides derived from the desired 5′ and 3′ positions of SEQ ID NO:1 may be designed to PCR amplify, and subsequently clone, the intended N- and/or C-terminal deletion mutant. Such primers could comprise, for example, an inititation and stop codon for the 5′ and 3′ primer, respectively. Such primers may also comprise restriction sites to facilitate cloning of the deletion mutant post amplification. Moreover, the primers may comprise additional sequences, such as, for example, flag-tag sequences, kozak sequences, or other sequences discussed and/or referenced herein.

Representative PCR amplification conditions are provided below, although the skilled artisan would appreciate that other conditions may be required for efficient amplification. A 100 ul PCR reaction mixture may be prepared using 10 ng of the template DNA (cDNA clone of BMSOTR), 200 uM 4dNTPs, 1 uM primers, 0.25U Taq DNA polymerase (PE), and standard Taq DNA polymerase buffer. Typical PCR cycling condition are as follows: 20-25 cycles of: (45 sec, 93 degrees; 2 min, 50 degrees; 2 min, 72 degrees) and 1 cycle of: (10 min, 72 degrees). After the final extension step of PCR, 5U Klenow Fragment may be added and incubated for 15 min at 30 degrees.

Upon digestion of the fragment with the NotI and SalI restriction enzymes, the fragment could be cloned into an appropriate expression and/or cloning vector which has been similarly digested (e.g., pSport1, among others). The skilled artisan would appreciate that other plasmids could be equally substituted, and may be desirable in certain circumstances. The digested fragment and vector are then ligated using a DNA ligase, and then used to transform competent E. coli cells using methods provided herein and/or otherwise known in the art.

The 5′ primer sequence for amplifying any additional N-terminal deletion mutants may be determined by reference to the following formula: (S+(X*3)) to ((S+(X*3))+25), wherein ‘S’ is equal to the nucleotide position of the initiating start codon of the BMSOTR gene (SEQ ID NO:1), and ‘X’ is equal to the most N-terminal amino acid of the intended N-terminal deletion mutant. The first term will provide the start 5′ nucleotide position of the 5′ primer, while the second term will provide the end 3′ nucleotide position of the 5′ primer corresponding to sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 5′ primer may be desired in certain circumstances (e.g., kozak sequences, etc.).

The 3′ primer sequence for amplifying any additional N-terminal deletion mutants may be determined by reference to the following formula: (S+(X*3)) to ((S+(X*3))−25), wherein ‘S’ is equal to the nucleotide position of the initiating start codon of the BMSOTR gene (SEQ ID NO:1), and ‘X’ is equal to the most C-terminal amino acid of the intended N-terminal deletion mutant. The first term will provide the start 5′ nucleotide position of the 3′ primer, while the second term will provide the end 3′ nucleotide position of the 3′ primer corresponding to the anti-sense strand of SEQ ID NO:1. Once the corresponding nucleotide positions of the primer are determined, the final nucleotide sequence may be created by the addition of applicable restriction site sequences to the 5′ end of the sequence, for example. As referenced herein, the addition of other sequences to the 3′ primer may be desired in certain circumstances (e.g., stop codon sequences, etc.). The skilled artisan would appreciate that modifications of the above nucleotide positions may be necessary for optimizing PCR amplification.

The same general formulas provided above may be used in identifying the 5′ and 3′ primer sequences for amplifying any C-terminal deletion mutant of the present invention. Moreover, the same general formulas provided above may be used in identifying the 5′ and 3′ primer sequences for amplifying any combination of N-terminal and C-terminal deletion mutant of the present invention. The skilled artisan would appreciate that modifications of the above nucleotide positions may be necessary for optimizing PCR amplification.

### Example 10

### Production of an Antibody from a Polypeptide

The antibodies of the present invention can be prepared by a variety of methods. (See, Current Protocols, Chapter 2.) As one example of such methods, cells expressing a polypeptide of the present invention are administered to an animal to induce the production of sera containing polyclonal antibodies. In a preferred method, a preparation of the protein is prepared and purified to render it substantially free of natural contaminants. Such a preparation is then introduced into an animal in order to produce polyclonal antisera of greater specific activity.

In the most preferred method, the antibodies of the present invention are monoclonal antibodies (or protein binding fragments thereof). Such monoclonal antibodies can be prepared using hybridoma technology. (Köhler et al., Nature 256:495 (1975); Köhler et al., Eur. J. Immunol. 6:511 (1976); Köhler et al., Eur. J. Immunol. 6:292 (1976); Hammerling et al., in: Monoclonal Antibodies and T-Cell Hybridomas, Elsevier, N.Y., pp. 563-681 (1981).) In general, such procedures involve immunizing an animal (preferably a mouse) with polypeptide or, more preferably, with a polypeptide-expressing cell. Such cells may be cultured in any suitable tissue culture medium; however, it is preferable to culture cells in Earle's modified Eagle's medium supplemented with 10% fetal bovine serum (inactivated at about 56 degrees C.), and supplemented with about 10 g/l of nonessential amino acids, about 1,000 U/ml of penicillin, and about 100 ug/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981).) The hybridoma cells obtained through such a selection are then assayed to identify clones which secrete antibodies capable of binding the polypeptide.

Alternatively, additional antibodies capable of binding to the polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones that produce an antibody whose ability to bind to the protein-specific antibody can be blocked by the polypeptide. Such antibodies comprise anti-idiotypic antibodies to the protein-specific antibody and can be used to immunize an animal to induce formation of further protein-specific antibodies.

It will be appreciated that Fab and F(ab′)2 and other fragments of the antibodies of the present invention may be used according to the methods disclosed herein. Such fragments are typically produced by proteolytic cleavage, using enzymes such as papain (to produce Fab fragments) or pepsin (to produce F(ab′)2 fragments). Alternatively, protein-binding fragments can be produced through the application of recombinant DNA technology or through synthetic chemistry.

For in vivo use of antibodies in humans, it may be preferable to use “humanized” chimeric monoclonal antibodies. Such antibodies can be produced using genetic constructs derived from hybridoma cells producing the monoclonal antibodies described above. Methods for producing chimeric antibodies are known in the art. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

Moreover, in another preferred method, the antibodies directed against the polypeptides of the present invention may be produced in plants. Specific methods are disclosed in U.S. Pat. Nos. 5,959,177, and 6,080,560, which are hereby incorporated in their entirety herein. The methods not only describe methods of expressing antibodies, but also the means of assembling foreign multimeric proteins in plants (i.e., antibodies, etc,), and the subsequent secretion of such antibodies from the plant.

### Example 34

### Production Of An Antibody

a) Hybridoma Technology

The antibodies of the present invention can be prepared by a variety of methods. (See, Current Protocols, Chapter 2.) As one example of such methods, cells expressing BMSOTR and/or BMSORT variant are administered to an animal to induce the production of sera containing polyclonal antibodies. In a preferred method, a preparation of BMSOTR and/or BMSORT variant protein is prepared and purified to render it substantially free of natural contaminants. Such a preparation is then introduced into an animal in order to produce polyclonal antisera of greater specific activity.

Monoclonal antibodies specific for protein BMSOTR and/or BMSORT variant are prepared using hybridoma technology. (Kohler et al., Nature 256:495 (1975); Kohler et al., Eur. J. Immunol. 6:511 (1976); Kohler et al., Eur. J. Immunol. 6:292 (1976); Hammerling et al., in: Monoclonal Antibodies and T-Cell Hybridomas, Elsevier, N.Y., pp. 563-681 (1981)). In general, an animal (preferably a mouse) is immunized with BMSOTR and/or BMSORT variant polypeptide or, more preferably, with a secreted BMSOTR and/or BMSORT variant polypeptide-expressing cell. Such polypeptide-expressing cells are cultured in any suitable tissue culture medium, preferably in Earle's modified Eagle's medium supplemented with 10% fetal bovine serum (inactivated at about 56° C.), and supplemented with about 10 g/l of nonessential amino acids, about 1,000 U/ml of penicillin, and about 100 μg/ml of streptomycin.

The splenocytes of such mice are extracted and fused with a suitable myeloma cell line. Any suitable myeloma cell line may be employed in accordance with the present invention; however, it is preferable to employ the parent myeloma cell line (SP20), available from the ATCC. After fusion, the resulting hybridoma cells are selectively maintained in HAT medium, and then cloned by limiting dilution as described by Wands et al. (Gastroenterology 80:225-232 (1981)). The hybridoma cells obtained through such a selection are then assayed to identify clones which secrete antibodies capable of binding the BMSOTR and/or BMSORT variant polypeptide.

Alternatively, additional antibodies capable of binding to BMSOTR and/or BMSORT variant polypeptide can be produced in a two-step procedure using anti-idiotypic antibodies. Such a method makes use of the fact that antibodies are themselves antigens, and therefore, it is possible to obtain an antibody that binds to a second antibody. In accordance with this method, protein specific antibodies are used to immunize an animal, preferably a mouse. The splenocytes of such an animal are then used to produce hybridoma cells, and the hybridoma cells are screened to identify clones which produce an antibody whose ability to bind to the BMSOTR and/or BMSORT variant protein-specific antibody can be blocked by BMSOTR and/or BMSORT variant. Such antibodies comprise anti-idiotypic antibodies to the BMSOTR and/or BMSORT variant protein-specific antibody and are used to immunize an animal to induce formation of further BMSOTR and/or BMSORT variant protein-specific antibodies.

For in vivo use of antibodies in humans, an antibody is “humanized”. Such antibodies can be produced using genetic constructs derived from hybridoma cells producing the monoclonal antibodies described above. Methods for producing chimeric and humanized antibodies are known in the art and are discussed herein. (See, for review, Morrison, Science 229:1202 (1985); Oi et al., BioTechniques 4:214 (1986); Cabilly et al., U.S. Pat. No. 4,816,567; Taniguchi et al., EP 171496; Morrison et al., EP 173494; Neuberger et al., WO 8601533; Robinson et al., WO 8702671; Boulianne et al., Nature 312:643 (1984); Neuberger et al., Nature 314:268 (1985).)

b) Isolation of Antibody Fragments Directed Against BMSOTR and/or BMSORT Variant from a Library of scFvs

Naturally occurring V-genes isolated from human PBLs are constructed into a library of antibody fragments which contain reactivities against BMSOTR and/or BMSORT variant to which the donor may or may not have been exposed (see e.g., U.S. Pat. No. 5,885,793 incorporated herein by reference in its entirety).

Rescue of the Library. A library of scFvs is constructed from the RNA of human PBLs as described in PCT publication WO 92/01047. To rescue phage displaying antibody fragments, approximately 109 E. coli harboring the phagemid are used to inoculate 50 ml of 2×TY containing 1% glucose and 100 μg/ml of ampicillin (2×TY-AMP-GLU) and grown to an O.D. of 0.8 with shaking. Five ml of this culture is used to inoculate 50 ml of 2×TY-AMP-GLU, 2×10⁸ TU of delta gene 3 helper (M13 delta gene III, see PCT publication WO 92/01047) are added and the culture incubated at 37° C. for 45 minutes without shaking and then at 37° C. for 45 minutes with shaking. The culture is centrifuged at 4000 r.p.m. for 10 min. and the pellet resuspended in 2 liters of 2×TY containing 100 μg/ml ampicillin and 50 ug/ml kanamycin and grown overnight. Phage are prepared as described in PCT publication WO 92/01047.

M13 delta gene III is prepared as follows: M13 delta gene III helper phage does not encode gene III protein, hence the phage(mid) displaying antibody fragments have a greater avidity of binding to antigen. Infectious M13 delta gene III particles are made by growing the helper phage in cells harboring a pUC19 derivative supplying the wild type gene III protein during phage morphogenesis. The culture is incubated for 1 hour at 37° C. without shaking and then for a further hour at 37° C. with shaking. Cells are spun down (IEC-Centra 8,400 r.p.m. for 10 min), resuspended in 300 ml 2×TY broth containing 100 μg ampicillin/ml and 25 μg kanamycin/ml (2×TY-AMPKAN) and grown overnight, shaking at 37° C. Phage particles are purified and concentrated from the culture medium by two PEG-precipitations (Sambrook et al., 1990), resuspended in 2 ml PBS and passed through a 0.45 μm filter (Minisart NML; Sartorius) to give a final concentration of approximately 1013 transducing units/ml (ampicillin-resistant clones).

Panning of the Library. Immunotubes (Nunc) are coated overnight in PBS with 4 ml of either 100 μg/ml or 10 μg/ml of a polypeptide of the present invention. Tubes are blocked with 2% Marvel-PBS for 2 hours at 37° C. and then washed 3 times in PBS. Approximately 1013 TU of phage is applied to the tube and incubated for 30 minutes at room temperature tumbling on an over and under turntable and then left to stand for another 1.5 hours. Tubes are washed 10 times with PBS 0.1% Tween-20 and 10 times with PBS. Phage are eluted by adding 1 ml of 100 mM triethylamine and rotating 15 minutes on an under and over turntable after which the solution is immediately neutralized with 0.5 ml of 1.0M Tris-HCl, pH 7.4. Phage are then used to infect 10 ml of mid-log E. coli T11 by incubating eluted phage with bacteria for 30 minutes at 37° C. The E. coli are then plated on TYE plates containing 1% glucose and 100 μg/ml ampicillin. The resulting bacterial library is then rescued with delta gene 3 helper phage as described above to prepare phage for a subsequent round of selection. This process is then repeated for a total of 4 rounds of affinity purification with tube-washing increased to 20 times with PBS, 0.1% Tween-20 and 20 times with PBS for rounds 3 and 4.

Characterization of Binders. Eluted phage from the 3rd and 4th rounds of selection are used to infect E. coli HB 2151 and soluble scFv is produced (Marks, et al., 1991) from single colonies for assay. ELISAs are performed with microtitre plates coated with either 10 pg/ml of the polypeptide of the present invention in 50 mM bicarbonate pH 9.6. Clones positive in ELISA are further characterized by PCR fingerprinting (see, e.g., PCT publication WO 92/01047) and then by sequencing. These ELISA positive clones may also be further characterized by techniques known in the art, such as, for example, epitope mapping, binding affinity, receptor signal transduction, ability to block or competitively inhibit antibody/antigen binding, and competitive agonistic or antagonistic activity.

### Example 12

### Identification and Cloning of VH and VL Domains of Antibodies Directed Against the BMSOTR and/or BMSORT Variant Polypeptide

VH and VL domains may be identified and cloned from cell lines expressing an antibody directed against a BMSOTR and/or BMSORT variant epitope by performing PCR with VH and VL specific primers on cDNA made from the antibody expressing cell lines. Briefly, RNA is isolated from the cell lines and used as a template for RT-PCR designed to amplify the VH and VL domains of the antibodies expressed by the EBV cell lines. Cells may be lysed using the TRIzol reagent (Life Technologies, Rockville, Md.) and extracted with one fifth volume of chloroform. After addition of chloroform, the solution is allowed to incubate at room temperature for 10 minutes, and then centrifuged at 14, 000 rpm for 15 minutes at 4 C in a tabletop centrifuge. The supernatant is collected and RNA is precipitated using an equal volume of isopropanol. Precipitated RNA is pelleted by centrifuging at 14, 000 rpm for 15 minutes at 4 C in a tabletop centrifuge.

Following centrifugation, the supernatant is discarded and washed with 75% ethanol. Follwing the wash step, the RNA is centrifuged again at 800 rpm for 5 minutes at 4 C. The supernatant is discarded and the pellet allowed to air dry. RNA is the dissolved in DEPC water and heated to 60 C for 10 minutes. Quantities of RNA can be determined using optical density measurements. cDNA may be synthesized, according to methods well-known in the art and/or described herein, from 1.5-2.5 micrograms of RNA using reverse transciptase and random hexamer primers. cDNA is then used as a template for PCR amplification of VH and VL domains.

Primers used to amplify VH and VL genes are shown below. Typically a PCR reaction makes use of a single 5′primer and a single 3′primer. Sometimes, when the amount of available RNA template is limiting, or for greater efficiency, groups of 5′ and/or 3′primers may be used. For example, sometimes all five VH-5′primers and all JH3′primers are used in a single PCR reaction. The PCR reaction is carried out in a 50 microliter volume containing 1× PCR buffer, 2 mM of each dNTP, 0.7 units of High Fidelity Taq polymerse, 5′primer mix, 3′primer mix and 7.5 microliters of cDNA. The 5′ and 3′primer mix of both VH and VL can be made by pooling together 22 pmole and 28 pmole, respectively, of each of the individual primers. PCR conditions are: 96 C for 5 minutes; followed by 25 cycles of 94 C for 1 minute, 50 C for 1 minute, and 72 C for 1 minute; followed by an extension cycle of 72 C for 10 minutes. After the reaction has been completed, sample tubes may be stored at 4 C.

Primer Sequences Used to Amplify VH domains

| Primer name | Primer Sequence | SEQ ID NO: |
|---|---|---|
| Hu VH1-5′ | CAGGTGCAGCTGGTGCAGTCTGG | 15 |
| Hu VH2-5′ | CAGGTCAACTTAAGGGAGTCTGG | 16 |
| Hu VH3-5′ | GAGGTGCAGCTGGTGGAGTCTGG | 17 |
| Hu VH4-5′ | CAGGTGCAGCTGCAGGAGTCGGG | 18 |
| Hu VH5-5′ | GAGGTGCAGCTGTTGCAGTCTGC | 19 |
| Hu VH6-5′ | CAGGTACAGCTGCAGCAGTCAGG | 20 |
| Hu JH1-5′ | TGAGGAGACGGTGACCAGGGTGCC | 21 |
| Hu JH3-5′ | TGAAGAGACGGTGACCATTGTCCC | 22 |
| Hu JH4-5′ | TGAGGAGACGGTGACCAGGGTTCC | 23 |
| Hu JH6-5′ | TGAGGAGACGGTGACCGTGGTCCC | 24 |

Primer Sequences Used to Amplify VL domains

| | | SEQ ID |
|---|---|---|
| Primer name | Primer Sequence | NO: |
| Hu Vkappa1-5′ | GACATCCAGATGACCCAGTCTCC | 25 |
| Hu Vkappa2a-5′ | GATGTTGTGATGACTCAGTCTCC | 26 |
| Hu Vkappa2b-5′ | GATATTGTGATGACTCAGTCTCC | 27 |
| Hu Vkappa3-5′ | GAAATTGTGTTGACGCAGTCTCC | 28 |
| Hu Vkappa4-5′ | GACATCGTGATGACCCAGTCTCC | 29 |
| Hu Vkappa5-5′ | GAAACGACACTCACGCAGTCTCC | 30 |
| Hu Vkappa6-5′ | GAAATTGTGCTGACTCAGTCTCC | 31 |
| Hu Vlambda1-5′ | CAGTCTGTGTTGACGCAGCCGCC | 32 |
| Hu Vlambda2-5′ | CAGTCTGCCCTGACTCAGCCTGC | 33 |
| Hu Vlambda3-5′ | TCCTATGTGCTGACTCAGCCACC | 34 |
| Hu Vlambda3b-5′ | TCTTCTGAGCTGACTCAGGACCC | 35 |
| Hu Vlambda4-5′ | CACGTTATACTGACTCAACCGCC | 36 |
| Hu Vlambda5-5′ | CAGGCTGTGCTCACTCAGCCGTC | 37 |
| Hu Vlambda6-5′ | AATTTTATGCTGACTCAGCCCCA | 38 |
| Hu Jkappa1-3′ | ACGTTTGATTTCCACCTTGGTCCC | 39 |
| Hu Jkappa2-3′ | ACGTTTGATCTCCAGCTTGGTCCC | 40 |
| Hu Jkappa3-3′ | ACGTTTGATATCCACTTTGGTCCC | 41 |
| Hu Jkappa4-3′ | ACGTTTGATCTCCACCTTGGTCCC | 42 |
| Hu Jkappa5-3′ | ACGTTTAATCTCCAGTCGTGTCCC | 43 |
| Hu Vlambda1-3′ | CAGTCTGTGTTGACGCAGCCGCC | 44 |
| Hu Vlambda2-3′ | CAGTCTGCCCTGACTCAGCCTGC | 45 |
| Hu Vlambda3-3′ | TCCTATGTGCTGACTCAGCCACC | 46 |
| Hu Vlambda3b-3′ | TCTTCTGAGCTGACTCAGGACCC | 47 |
| Hu Vlambda4-3′ | CACGTTATACTGACTCAACCGCC | 48 |
| Hu Vlambda5-3′ | CAGGCTGTGCTCACTCAGCCGTC | 49 |
| Hu Vlambda6-3′ | AATTTTATGCTGACTCAGCCCCA | 50 |

PCR samples are then electrophoresed on a 1.3% agarose gel. DNA bands of the expected sizes (−506 base pairs for VH domains, and 344 base pairs for VL domains) can be cut out of the gel and purified using methods well known in the art and/or described herein.

Purified PCR products can be ligated into a PCR cloning vector (TA vector from Invitrogen Inc., Carlsbad, Calif.). Individual cloned PCR products can be isolated after transfection of E. coli and blue/white color selection. Cloned PCR products may then be sequenced using methods commonly known in the art and/or described herein.

The PCR bands containing the VH domain and the VL domains can also be used to create full-length Ig expression vectors. VH and VL domains can be cloned into vectors containing the nucleotide sequences of a heavy (e.g., human IgG1 or human IgG4) or light chain (human kappa or human ambda) constant regions such that a complete heavy or light chain molecule could be expressed from these vectors when transfected into an appropriate host cell. Further, when cloned heavy and light chains are both expressed in one cell line (from either one or two vectors), they can assemble into a complete functional antibody molecule that is secreted into the cell culture medium. Methods using polynucleotides encoding VH and VL antibody domain to generate expression vectors that encode complete antibody molecules are well known within the art.

### Example 14

### Method of Screening, In Vitro, Compounds that Bind to the BMSOTR and/or BMSORT Variant Polypeptide

In vitro systems can be designed to identify compounds capable of binding the BMSOTR and/or BMSORT variant polypeptide of the invention. Compounds identified can be useful, for example, in modulating the activity of wild type and/or mutant BMSOTR and/or BMSORT variant polypeptide, preferably mutant BMSOTR and/or BMSORT variant polypeptide, can be useful in elaborating the biological function of the BMSOTR and/or BMSORT variant polypeptide, can be utilized in screens for identifying compounds that disrupt normal BMSOTR and/or BMSORT variant polypeptide interactions, or can in themselves disrupt such interactions.

The principle of the assays used to identify compounds that bind to the BMSOTR and/or BMSORT variant polypeptide involves preparing a reaction mixture of the BMSOTR and/or BMSORT variant polypeptide and the test compound under conditions and for a time sufficient to allow the two components to interact and bind, thus forming a complex which can be removed and/or detected in the reaction mixture. These assays can be conducted in a variety of ways. For example, one method to conduct such an assay would involve anchoring BMSOTR and/or BMSORT variant polypeptide or the test substance onto a solid phase and detecting BMSOTR and/or BMSORT variant polypeptide/test compound complexes anchored on the solid phase at the end of the reaction. In one embodiment of such a method, the BMSOTR and/or BMSORT variant polypeptide can be anchored onto a solid surface, and the test compound, which is not anchored, can be labeled, either directly or indirectly.

In practice, microtitre plates can conveniently be utilized as the solid phase. The anchored component can be immobilized by non-covalent or covalent attachments. Non-covalent attachment can be accomplished by simply coating the solid surface with a solution of the protein and drying. Alternatively, an immobilized antibody, preferably a monoclonal antibody, specific for the protein to be immobilized can be used to anchor the protein to the solid surface. The surfaces can be prepared in advance and stored.

In order to conduct the assay, the nonimmobilized component is added to the coated surface containing the anchored component. After the reaction is complete, unreacted components are removed (e.g., by washing) under conditions such that any complexes formed will remain immobilized on the solid surface. The detection of complexes anchored on the solid surface can be accomplished in a number of ways. Where the previously immobilized component is pre-labeled, the detection of label immobilized on the surface indicates that complexes were formed. Where the previously nonimmobilized component is not pre-labeled, an indirect label can be used to detect complexes anchored on the surface; e.g., using a labeled antibody specific for the immobilized component (the antibody, in turn, can be directly labeled or indirectly labeled with a labeled anti-Ig antibody).

Alternatively, a reaction can be conducted in a liquid phase, the reaction products separated from unreacted components, and complexes detected; e.g., using an immobilized antibody specific for BMSOTR and/or BMSORT variant polypeptide or the test compound to anchor any complexes formed in solution, and a labeled antibody specific for the other component of the possible complex to detect anchored complexes.

Another example of a screening assay to identify compounds that bind to BMSOTR and/or BMSORT variant, relates to the application of a cell membrane-based scintillation proximity assay (“SPA”). Such an assay would require the idenification of a ligand for BMSOTR and/or BMSORT variant polypeptide. Once identified, unlabeled ligand is added to assay-ready plates that would serve as a positive control. The SPA beads and membranes are added next, and then ¹²⁵I-labeled ligand is added. After an equilibration period of 2-4 hours at room temperature, the plates can be counted in a scintillation counting machine, and the percent inhibition or stimulation calculated. Such an SPA assay may be based upon a manual, automated, or semi-automated platform, and encompass 96, 384, 1536-well plates or more. Any number of SPA beads may be used as applicable to each assay. Examples of SPA beads include, for example, Leadseeker WGA PS (Amersham cat # RPNQ 0260), and SPA Beads (PVT-PEI-WGA-TypeA; Amersham cat # RPNQ0003). The utilized membranes may also be derived from a number of cell line and tissue sources depending upon the expression profile of the respective polypeptide and the adaptability of such a cell line or tissue source to the development of a SPA-based assay. Examples of membrane preparations include, for example, cell lines transformed to express the receptor to be assayed in CHO cells or HEK cells, for example. SPA-based assays are well known in the art and are encompassed by the present invention. One such assay is described in U.S. Pat. No. 4,568,649, which is incorporated herein by reference. The skilled artisan would acknowledge that certain modifications of known SPA assays may be required to adapt such assays to each respective polypeptide.

One such screening procedure involves the use of melanophores which are transfected to express the BMSOTR and/or BMSORT variant polypeptide of the present invention. Such a screening technique is described in PCT WO 92/01810, published Feb. 6, 1992. Such an assay may be employed to screen for a compound which inhibits activation of the receptor polypeptide of the present invention by contacting the melanophore cells which encode the receptor with both the receptor ligand, such as LPA, and a compound to be screened. Inhibition of the signal generated by the ligand indicates that a compound is a potential antagonist for the receptor, i.e., inhibits activation of the receptor.

The technique may also be employed for screening of compounds which activate the receptor by contacting such cells with compounds to be screened and determining whether such compound generates a signal, i.e., activates the receptor. Other screening techniques include the use of cells which express the BMSOTR and/or BMSORT variant polypeptide (for example, transfected CHO cells) in a system which measures extracellular pH changes caused by receptor activation. In this technique, compounds may be contacted with cells expressing the receptor polypeptide of the present invention. A second messenger response, e.g., signal transduction or pH changes, is then measured to determine whether the potential compound activates or inhibits the receptor.

Another screening technique involves expressing the BMSOTR and/or BMSORT variant polypeptide in which the receptor is linked to phospholipase C or D. Representative examples of such cells include, but are not limited to, endothelial cells, smooth muscle cells, and embryonic kidney cells. The screening may be accomplished as hereinabove described by detecting activation of the receptor or inhibition of activation of the receptor from the phospholipase second signal.

Another method involves screening for compounds which are antagonists or agonists by determining inhibition of binding of labeled ligand, such as LPA, to cells which have the receptor on the surface thereof, or cell membranes containing the receptor. Such a method involves transfecting a cell (such as eukaryotic cell) with DNA encoding the BMSOTR and/or BMSORT variant polypeptide such that the cell expresses the receptor on its surface. The cell is then contacted with a potential antagonist or agonist in the presence of a labeled form of a ligand, such as LPA. The ligand can be labeled, e.g., by radioactivity. The amount of labeled ligand bound to the receptors is measured, e.g., by measuring radioactivity associated with transfected cells or membrane from these cells. If the compound binds to the receptor, the binding of labeled ligand to the receptor is inhibited as determined by a reduction of labeled ligand which binds to the receptors. This method is called binding assay.

Another screening procedure involves the use of mammalian cells (CHO, HEK 293, Xenopus oocytes, RBL-2H3, etc) which are transfected to express the receptor of interest. The cells are loaded with an indicator dye that produces a fluorescent signal when bound to calcium, and the cells are contacted with a test substance and a receptor agonist, such as LPA. Any change in fluorescent signal is measured over a defined period of time using, for example, a fluorescence spectrophotometer or a fluorescence imaging plate reader. A change in the fluorescence signal pattern generated by the ligand indicates that a compound is a potential antagonist or agonist for the receptor.

Another screening procedure involves use of mammalian cells (CHO, HEK293, Xenopus oocytes, RBL-2H3, etc.) which are transfected to express the receptor of interest, and which are also transfected with a reporter gene construct that is coupled to activation of the receptor (for example, luciferase or beta-galactosidase behind an appropriate promoter). The cells are contacted with a test substance and the receptor agonist (ligand), such as LPA, and the signal produced by the reporter gene is measured after a defined period of time. The signal can be measured using a luminometer, spectrophotometer, fluorimeter, or other such instrument appropriate for the specific reporter construct used. Change of the signal generated by the ligand indicates that a compound is a potential antagonist or agonist for the receptor.

Another screening technique for antagonists or agonits involves introducing RNA encoding the BMSOTR and/or BMSORT variant polypeptide into Xenopus oocytes (or CHO, HEK 293, RBL-2H3, etc.) to transiently or stably express the receptor. The receptor oocytes are then contacted with the receptor ligand, such as LPA, and a compound to be screened. Inhibition or activation of the receptor is then determined by detection of a signal, such as, cAMP, calcium, proton, or other ions.

Another method involves screening for BMSOTR and/or BMSORT variant polypeptide inhibitors by determining inhibition or stimulation of BMSOTR and/or BMSORT variant polypeptide-mediated cAMP and/or adenylate cyclase accumulation or dimunition. Such a method involves transiently or stably transfecting a eukaryotic cell with BMSOTR and/or BMSORT variant polypeptide receptor to express the receptor on the cell surface.

The cell is then exposed to potential antagonists or agonists in the presence of BMSOTR and/or BMSORT variant polypeptide ligand, such as LPA. The changes in levels of cAMP is then measured over a defined period of time, for example, by radio-immuno or protein binding assays (for example using Flashplates or a scintillation proximity assay). Changes in cAMP levels can also be determined by directly measuring the activity of the enzyme, adenylyl cyclase, in broken cell preparations. If the potential antagonist or agonist binds the receptor, and thus inhibits BMSOTR and/or BMSORT variant polypeptide-ligand binding, the levels of BMSOTR and/or BMSORT variant polypeptide-mediated cAMP, or adenylate cyclase activity, will be reduced or increased.

One preferred screening method involves co-transfecting HEK-293 cells with a mammalian expression plasmid encoding a G-protein coupled receptor (GPCR), such as BMSOTR and/or BMSORT variant, along with a mixture comprised of mammalian expression plasmids cDNAs encoding GU15 (Wilkie T. M. et al Proc Natl Acad Sci USA 1991 88: 10049-10053), GU16 (Amatruda T. T. et al Proc Natl Acad Sci USA 1991 8: 5587-5591, and three chimeric G-proteins refered to as Gqi5, Gqs5, and Gqo5 (Conklin B R et al Nature 1993 363: 274-276, Conklin B. R. et al Mol Pharmacol 1996 50: 885-890). Following a 24 h incubation the trasfected HEK-293 cells are plated into poly-D-lysine coated 96 well black/clear plates (Becton Dickinson, Bedford, Mass.).

The cells are assayed on FLIPR (Fluorescent Imaging Plate Reader, Molecular Devices, Sunnyvale, Calif.) for a calcium mobilization response following addition of test ligands. Upon identification of a ligand which stimulates calcium mobilization in HEK-293 cells expressing a given GPCR and the G-protein mixtures, subsequent experiments are performed to determine which, if any, G-protein is required for the functional response. HEK-293 cells are then transfected with the test GPCR, or co-transfected with the test GPCR and G015, GD16, GqiS, Gqs5, or Gqo5. If the GPCR requires the presence of one of the G-proteins for functional expression in HEK-293 cells, all subsequent experiments are performed with HEK-293 cell cotransfected with the GPCR and the G-protein which gives the best response. Alternatively, the receptor can be expressed in a different cell line, for example RBL-2H3, without additional Gproteins.

Another screening method for agonists and antagonists relies on the endogenous pheromone response pathway in the yeast, Saccharomyces cerevisiae. Heterothallic strains of yeast can exist in two mitotically stable haploid mating types, MATa and MATa. Each cell type secretes a small peptide hormone that binds to a G-protein coupled receptor on opposite mating type cells which triggers a MAP kinase cascade leading to G1 arrest as a prelude to cell fusion.

Genetic alteration of certain genes in the pheromone response pathway can alter the normal response to pheromone, and heterologous expression and coupling of human G-protein coupled receptors and humanized G-protein subunits in yeast cells devoid of endogenous pheromone receptors can be linked to downstream signaling pathways and reporter genes (e.g., U.S. Pat. Nos. 5,063,154; 5,482,835; 5,691,188). Such genetic alterations include, but are not limited to, (i) deletion of the STE2 or STE3 gene encoding the endogenous G-protein coupled pheromone receptors; (ii) deletion of the FAR1 gene encoding a protein that normally associates with cyclindependent kinases leading to cell cycle arrest; and (iii) construction of reporter genes fused to the FUS 1 gene promoter (where FUS 1 encodes a membrane-anchored glycoprotein required for cell fusion). Downstream reporter genes can permit either a positive growth selection (e.g., histidine prototrophy using the FUS1-HIS3 reporter), or a colorimetric, fluorimetric or spectrophotometric readout, depending on the specific reporter construct used (e.g., b-galactosidase induction using a FUS1-LacZ reporter).

The yeast cells can be further engineered to express and secrete small peptides from random peptide libraries, some of which can permit autocrine activation of heterologously expressed human (or mammalian) G-protein coupled receptors (Broach, J. R. and Thorner, J., Nature 384: 14-16, 1996; Manfredi et al., Mol. Cell. Biol. 16: 4700-4709,1996). This provides a rapid direct growth selection (e.g, using the FUS 1-HIS3 reporter) for surrogate peptide agonists that activate characterized or orphan receptors. Alternatively, yeast cells that functionally express human (or mammalian) G-protein coupled receptors linked to a reporter gene readout (e.g., FUS1-LacZ) can be used as a platform for high-throughput screening of known ligands, fractions of biological extracts and libraries of chemical compounds for either natural or surrogate ligands.

Functional agonists of sufficient potency (whether natural or surrogate) can be used as screening tools in yeast cell-based assays for identifying G-protein coupled receptor antagonists. For example, agonists will promote growth of a cell with FUS-HIS3 reporter or give positive readout for a cell with FUS1-LacZ. However, a candidate compound which inhibits growth or negates the positive readout induced by an agonist is an antagonist. For this purpose, the yeast system offers advantages over mammalian expression systems due to its ease of utility and null receptor background (lack of endogenous G-protein coupled receptors) which often interferes with the ability to identify agonists or antagonists.

### Example 15

### Bacterial Expression of a Polypeptide

A polynucleotide encoding a polypeptide of the present invention is amplified using PCR oligonucleotide primers corresponding to the 5′ and 3′ ends of the DNA sequence to synthesize insertion fragments. The primers used to amplify the cDNA insert should preferably contain restriction sites, such as BamHI and XbaI, at the 5′ end of the primers in order to clone the amplified product into the expression vector. For example, BamHI and XbaI correspond to the restriction enzyme sites on the bacterial expression vector pQE-9. (Qiagen, Inc., Chatsworth, Calif.). This plasmid vector encodes antibiotic resistance (Ampr), a bacterial origin of replication (ori), an IPTG-regulatable promoter/operator (P/O), a ribosome binding site (RBS), a 6-histidine tag (6-His), and restriction enzyme cloning sites.

The pQE-9 vector is digested with BamHI and XbaI and the amplified fragment is ligated into the pQE-9 vector maintaining the reading frame initiated at the bacterial RBS. The ligation mixture is then used to transform the E. coli strain M15/rep4 (Qiagen, Inc.) which contains multiple copies of the plasmid pREP4, that expresses the lacI repressor and also confers kanamycin resistance (Kanr). Transformants are identified by their ability to grow on LB plates and ampicillin/kanamycin resistant colonies are selected. Plasmid DNA is isolated and confirmed by restriction analysis.

Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.600) of between 0.4 and 0.6. IPTG (Isopropyl-B-D-thiogalacto pyranoside) is then added to a final concentration of 1 mM. IPTG induces by inactivating the lacI repressor, clearing the P/O leading to increased gene expression.

Cells are grown for an extra 3 to 4 hours. Cells are then harvested by centrifugation (20 mins at 6000×g). The cell pellet is solubilized in the chaotropic agent 6 Molar Guanidine HCl by stirring for 3-4 hours at 4 degree C. The cell debris is removed by centrifugation, and the supernatant containing the polypeptide is loaded onto a nickel-nitrilo-tri-acetic acid (“Ni-NTA”) affinity resin column (available from QIAGEN, Inc., supra). Proteins with a 6×His tag bind to the Ni-NTA resin with high affinity and can be purified in a simple one-step procedure (for details see: The QIAexpressionist (1995) QIAGEN, Inc., supra).

Briefly, the supernatant is loaded onto the column in 6 M guanidine-HCl, pH 8, the column is first washed with 10 volumes of 6 M guanidine-HCl, pH 8, then washed with 10 volumes of 6 M guanidine-HCl pH 6, and finally the polypeptide is eluted with 6 M guanidine-HCl, pH 5.

The purified protein is then renatured by dialyzing it against phosphate-buffered saline (PBS) or 50 mM Na-acetate, pH 6 buffer plus 200 mM NaCl. Alternatively, the protein can be successfully refolded while immobilized on the Ni-NTA column. The recommended conditions are as follows: renature using a linear 6M-1M urea gradient in 500 mM NaCl, 20% glycerol, 20 mM Tris/HCl pH 7.4, containing protease inhibitors. The renaturation should be performed over a period of 1.5 hours or more. After renaturation the proteins are eluted by the addition of 250 mM imidazole. Imidazole is removed by a final dialyzing step against PBS or 50 mM sodium acetate pH 6 buffer plus 200 mM NaCl. The purified protein is stored at 4 degree C. or frozen at −80 degree C.

### Example 16

### Purification of a Polypeptide from an Inclusion Body

The following alternative method can be used to purify a polypeptide expressed in E. coli when it is present in the form of inclusion bodies. Unless otherwise specified, all of the following steps are conducted at 4-10 degree C.

Upon completion of the production phase of the E. coli fermentation, the cell culture is cooled to 4-10 degree C. and the cells harvested by continuous centrifugation at 15,000 rpm (Heraeus Sepatech). On the basis of the expected yield of protein per unit weight of cell paste and the amount of purified protein required, an appropriate amount of cell paste, by weight, is suspended in a buffer solution containing 100 mM Tris, 50 mM EDTA, pH 7.4. The cells are dispersed to a homogeneous suspension using a high shear mixer.

The cells are then lysed by passing the solution through a microfluidizer (Microfluidics, Corp. or APV Gaulin, Inc.) twice at 4000-6000 psi. The homogenate is then mixed with NaCl solution to a final concentration of 0.5 M NaCl, followed by centrifugation at 7000×g for 15 min. The resultant pellet is washed again using 0.5M NaCl, 100 mM Tris, 50 mM EDTA, pH 7.4.

The resulting washed inclusion bodies are solubilized with 1.5 M guanidine hydrochloride (GuHCl) for 2-4 hours. After 7000×g centrifugation for 15 min., the pellet is discarded and the polypeptide containing supernatant is incubated at 4 degree C. overnight to allow further GuHCl extraction.

Following high speed centrifugation (30,000×g) to remove insoluble particles, the GuHCl solubilized protein is refolded by quickly mixing the GuHCl extract with 20 volumes of buffer containing 50 mM sodium, pH 4.5, 150 mM NaCl, 2 mM EDTA by vigorous stirring. The refolded diluted protein solution is kept at 4 degree C. without mixing for 12 hours prior to further purification steps.

To clarify the refolded polypeptide solution, a previously prepared tangential filtration unit equipped with 0.16 um membrane filter with appropriate surface area (e.g., Filtron), equilibrated with 40 mM sodium acetate, pH 6.0 is employed. The filtered sample is loaded onto a cation exchange resin (e.g., Poros HS-50, Perceptive Biosystems). The column is washed with 40 mM sodium acetate, pH 6.0 and eluted with 250 mM, 500 mM, 1000 mM, and 1500 mM NaCl in the same buffer, in a stepwise manner. The absorbance at 280 nm of the effluent is continuously monitored. Fractions are collected and further analyzed by SDS-PAGE.

Fractions containing the polypeptide are then pooled and mixed with 4 volumes of water. The diluted sample is then loaded onto a previously prepared set of tandem columns of strong anion (Poros HQ-50, Perceptive Biosystems) and weak anion (Poros CM-20, Perceptive Biosystems) exchange resins. The columns are equilibrated with 40 mM sodium acetate, pH 6.0. Both columns are washed with 40 mM sodium acetate, pH 6.0, 200 mM NaCl. The CM-20 column is then eluted using a 10 column volume linear gradient ranging from 0.2 M NaCl, 50 mM sodium acetate, pH 6.0 to 1.0 M NaCl, 50 mM sodium acetate, pH 6.5. Fractions are collected under constant A280 monitoring of the effluent. Fractions containing the polypeptide (determined, for instance, by 16% SDS-PAGE) are then pooled.

The resultant polypeptide should exhibit greater than 95% purity after the above refolding and purification steps. No major contaminant bands should be observed from Coomassie blue stained 16% SDS-PAGE gel when 5 ug of purified protein is loaded. The purified protein can also be tested for endotoxin/LPS contamination, and typically the LPS content is less than 0.1 ng/ml according to LAL assays.

### Example 17

### Cloning and Expression of a Polypeptide in a Baculovirus Expression System

In this example, the plasmid shuttle vector pAc373 is used to insert a polynucleotide into a baculovirus to express a polypeptide. A typical baculovirus expression vector contains the strong polyhedrin promoter of the Autographa californica nuclear polyhedrosis virus (AcMNPV) followed by convenient restriction sites, which may include, for example BamHI, Xba I and Asp718. The polyadenylation site of the simian virus 40 (“SV40”) is often used for efficient polyadenylation. For easy selection of recombinant virus, the plasmid contains the beta-galactosidase gene from E. coli under control of a weak Drosophila promoter in the same orientation, followed by the polyadenylation signal of the polyhedrin gene. The inserted genes are flanked on both sides by viral sequences for cell-mediated homologous recombination with wild-type viral DNA to generate a viable virus that express the cloned polynucleotide.

Many other baculovirus vectors can be used in place of the vector above, such as pVL941 and pAcIM1, as one skilled in the art would readily appreciate, as long as the construct provides appropriately located signals for transcription, translation, secretion and the like, including a signal peptide and an in-frame AUG as required. Such vectors are described, for instance, in Luckow et al., Virology 170:31-39 (1989).

A polynucleotide encoding a polypeptide of the present invention is amplified using PCR oligonucleotide primers corresponding to the 5′ and 3′ ends of the DNA sequence, as outlined in Example 15, to synthesize insertion fragments. The primers used to amplify the cDNA insert should preferably contain restriction sites at the 5′ end of the primers in order to clone the amplified product into the expression vector. Specifically, the cDNA sequence contained in the deposited clone, including the AUG initiation codon and the naturally associated leader sequence identified elsewhere herein (if applicable), is amplified using the PCR protocol described in Example 15. If the naturally occurring signal sequence is used to produce the protein, the vector used does not need a second signal peptide. Alternatively, the vector can be modified to include a baculovirus leader sequence, using the standard methods described in Summers et al., “A Manual of Methods for Baculovirus Vectors and Insect Cell Culture Procedures” Texas Agricultural Experimental Station Bulletin No. 1555 (1987).

The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The plasmid is digested with the corresponding restriction enzymes and optionally, can be dephosphorylated using calf intestinal phosphatase, using routine procedures known in the art. The DNA is then isolated from a 1% agarose gel using a commercially available kit (“Geneclean” BIO 101 Inc., La Jolla, Calif.).

The fragment and the dephosphorylated plasmid are ligated together with T4 DNA ligase. E. coli HB101 or other suitable E. coli hosts such as XL-1 Blue (Stratagene Cloning Systems, La Jolla, Calif.) cells are transformed with the ligation mixture and spread on culture plates. Bacteria containing the plasmid are identified by digesting DNA from individual colonies and analyzing the digestion product by gel electrophoresis. The sequence of the cloned fragment is confirmed by DNA sequencing.

Five ug of a plasmid containing the polynucleotide is co-transformed with 1.0 ug of a commercially available linearized baculovirus DNA (“BaculoGold™ baculovirus DNA”, Pharmingen, San Diego, Calif.), using the lipofection method described by Felgner et al., Proc. Natl. Acad. Sci. USA 84:7413-7417 (1987). One ug of BaculoGold™ virus DNA and 5 ug of the plasmid are mixed in a sterile well of a microtiter plate containing 50 ul of serum-free Grace's medium (Life Technologies Inc., Gaithersburg, Md.). Afterwards, 10 ul Lipofectin plus 90 ul Grace's medium are added, mixed and incubated for 15 minutes at room temperature. Then the transfection mixture is added drop-wise to Sf9 insect cells (ATCC CRL 1711) seeded in a 35 mm tissue culture plate with 1 ml Grace's medium without serum. The plate is then incubated for 5 hours at 27 degrees C. The transfection solution is then removed from the plate and 1 ml of Grace's insect medium supplemented with 10% fetal calf serum is added. Cultivation is then continued at 27 degrees C. for four days.

After four days the supernatant is collected and a plaque assay is performed, as described by Summers and Smith, supra. An agarose gel with “Blue Gal” (Life Technologies Inc., Gaithersburg) is used to allow easy identification and isolation of gal-expressing clones, which produce blue-stained plaques. (A detailed description of a “plaque assay” of this type can also be found in the user's guide for insect cell culture and baculovirology distributed by Life Technologies Inc., Gaithersburg, page 9-10.) After appropriate incubation, blue stained plaques are picked with the tip of a micropipettor (e.g., Eppendorf). The agar containing the recombinant viruses is then resuspended in a microcentrifuge tube containing 200 ul of Grace's medium and the suspension containing the recombinant baculovirus is used to infect Sf9 cells seeded in 35 mm dishes. Four days later the supernatants of these culture dishes are harvested and then they are stored at 4 degree C.

To verify the expression of the polypeptide, Sf9 cells are grown in Grace's medium supplemented with 10% heat-inactivated FBS. The cells are infected with the recombinant baculovirus containing the polynucleotide at a multiplicity of infection (“MOI”) of about 2. If radiolabeled proteins are desired, 6 hours later the medium is removed and is replaced with SF900 II medium minus methionine and cysteine (available from Life Technologies Inc., Rockville, Md.). After 42 hours, 5 uCi of 35S-methionine and 5 uCi 35S-cysteine (available from Amersham) are added. The cells are further incubated for 16 hours and then are harvested by centrifugation. The proteins in the supernatant as well as the intracellular proteins are analyzed by SDSPAGE followed by autoradiography (if radiolabeled).

Microsequencing of the amino acid sequence of the amino terminus of purified protein may be used to determine the amino terminal sequence of the produced protein.

### Example 18

### Expression of a Polypeptide in Mammalian Cells

The polypeptide of the present invention can be expressed in a mammalian cell. A typical mammalian expression vector contains a promoter element, which mediates the initiation of transcription of mRNA, a protein coding sequence, and signals required for the termination of transcription and polyadenylation of the transcript. Additional elements include enhancers, Kozak sequences and intervening sequences flanked by donor and acceptor sites for RNA splicing. Highly efficient transcription is achieved with the early and late promoters from SV40, the long terminal repeats (LTRs) from Retroviruses, e.g., RSV, HTLVI, HIVI and the early promoter of the cytomegalovirus (CMV). However, cellular elements can also be used (e.g., the human actin promoter).

Suitable expression vectors for use in practicing the present invention include, for example, vectors such as pSVL and pMSG (Pharmacia, Uppsala, Sweden), pRSVcat (ATCC 37152), pSV2dhfr (ATCC 37146), pBC12MI (ATCC 67109), pCMVSport 2.0, and pCMVSport 3.0. Mammalian host cells that could be used include, human Hela, 293, H9 and Jurkat cells, mouse NIH3T3 and C127 cells, Cos 1, Cos 7 and CV1, quail QC1-3 cells, mouse L cells and Chinese hamster ovary (CHO) cells.

Alternatively, the polypeptide can be expressed in stable cell lines containing the polynucleotide integrated into a chromosome. The co-transformation with a selectable marker such as dhfr, gpt, neomycin, hygromycin allows the identification and isolation of the transformed cells.

The transformed gene can also be amplified to express large amounts of the encoded protein. The DHFR (dihydrofolate reductase) marker is useful in developing cell lines that carry several hundred or even several thousand copies of the gene of interest. (See, e.g., Alt, F. W., et al., J. Biol. Chem . . . 253:1357-1370 (1978); Hamlin, J. L. and Ma, C., Biochem. et Biophys. Acta, 1097:107-143 (1990); Page, M. J. and Sydenham, M. A., Biotechnology 9:64-68 (1991).) Another useful selection marker is the enzyme glutamine synthase (GS) (Murphy et al., Biochem J. 227:277-279 (1991); Bebbington et al., Bio/Technology 10:169-175 (1992). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of proteins.

A polynucleotide of the present invention is amplified according to the protocol outlined in herein. If the naturally occurring signal sequence is used to produce the protein, the vector does not need a second signal peptide. Alternatively, if the naturally occurring signal sequence is not used, the vector can be modified to include a heterologous signal sequence. (See, e.g., WO 96/34891.) The amplified fragment is isolated from a 1% agarose gel using a commercially available kit (“Geneclean” BIO 101 Inc., La Jolla, Calif.). The fragment then is digested with appropriate restriction enzymes and again purified on a 1% agarose gel.

The amplified fragment is then digested with the same restriction enzyme and purified on a 1% agarose gel. The isolated fragment and the dephosphorylated vector are then ligated with T4 DNA ligase. E. coli HB101 or XL-1 Blue cells are then transformed and bacteria are identified that contain the fragment inserted into plasmid pC6 using, for instance, restriction enzyme analysis.

Chinese hamster ovary cells lacking an active DHFR gene is used for transformation. Five μg of an expression plasmid is cotransformed with 0.5 ug of the plasmid pSVneo using lipofectin (Feigner et al., supra). The plasmid pSV2-neo contains a dominant selectable marker, the neo gene from Tn5 encoding an enzyme that confers resistance to a group of antibiotics including G418. The cells are seeded in alpha minus MEM supplemented with 1 mg/ml G418. After 2 days, the cells are trypsinized and seeded in hybridoma cloning plates (Greiner, Germany) in alpha minus MEM supplemented with 10, 25, or 50 ng/ml of methotrexate plus 1 mg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 uM, 2 uM, 5 uM, 10 mM, 20 mM). The same procedure is repeated until clones are obtained which grow at a concentration of 100-200 uM. Expression of the desired gene product is analyzed, for instance, by SDSPAGE and Western blot or by reversed phase HPLC analysis.

### Example 19

### Method of enhancing the Biological Activity/Functional Characteristics of Invention Through Molecular Evolution

Although many of the most biologically active proteins known are highly effective for their specified function in an organism, they often possess characteristics that make them undesirable for transgenic, therapeutic, pharmaceutical, and/or industrial applications. Among these traits, a short physiological half-life is the most prominent problem, and is present either at the level of the protein, or the level of the proteins mRNA. The ability to extend the half-life, for example, would be particularly important for a proteins use in gene therapy, transgenic animal production, the bioprocess production and purification of the protein, and use of the protein as a chemical modulator among others. Therefore, there is a need to identify novel variants of isolated proteins possessing characteristics which enhance their application as a therapeutic for treating diseases of animal origin, in addition to the proteins applicability to common industrial and pharmaceutical applications.

Thus, one aspect of the present invention relates to the ability to enhance specific characteristics of invention through directed molecular evolution. Such an enhancement may, in a non-limiting example, benefit the inventions utility as an essential component in a kit, the inventions physical attributes such as its solubility, structure, or codon optimization, the inventions specific biological activity, including any associated enzymatic activity, the proteins enzyme kinetics, the proteins Ki, Kcat, Km, Vmax, Kd, protein-protein activity, protein-DNA binding activity, antagonist/inhibitory activity (including direct or indirect interaction), agonist activity (including direct or indirect interaction), the proteins antigenicity (e.g., where it would be desirable to either increase or decrease the antigenic potential of the protein), the immunogenicity of the protein, the ability of the protein to form dimers, trimers, or multimers with either itself or other proteins, the antigenic efficacy of the invention, including its subsequent use a preventative treatment for disease or disease states, or as an effector for targeting diseased genes. Moreover, the ability to enhance specific characteristics of a protein may also be applicable to changing the characterized activity of an enzyme to an activity completely unrelated to its initially characterized activity. Other desirable enhancements of the invention would be specific to each individual protein, and would thus be well known in the art and contemplated by the present invention.

For example, an engineered G-protein coupled receptor may be constitutively active upon binding of its cognate ligand. Alternatively, an engineered G-protein coupled receptor may be constitutively active in the absence of ligand binding. In yet another example, an engineered GPCR may be capable of being activated with less than all of the regulatory factors and/or conditions typically required for GPCR activation (e.g., ligand binding, phosphorylation, conformational changes, etc.). Such GPCRs would be useful in screens to identify GPCR modulators, among other uses described herein.

Directed evolution is comprised of several steps. The first step is to establish a library of variants for the gene or protein of interest. The most important step is to then select for those variants that entail the activity you wish to identify. The design of the screen is essential since your screen should be selective enough to eliminate non-useful variants, but not so stringent as to eliminate all variants. The last step is then to repeat the above steps using the best variant from the previous screen. Each successive cycle, can then be tailored as necessary, such as increasing the stringency of the screen, for example.

Over the years, there have been a number of methods developed to introduce mutations into macromolecules. Some of these methods include, random mutagenesis, “error-prone” PCR, chemical mutagenesis, site-directed mutagenesis, and other methods well known in the art (for a comprehensive listing of current mutagenesis methods, see Maniatis, Molecular Cloning: A Laboratory Manual, Cold Spring Harbor Press, Cold Spring, N.Y. (1982)). Typically, such methods have been used, for example, as tools for identifying the core functional region(s) of a protein or the function of specific domains of a protein (if a multi-domain protein). However, such methods have more recently been applied to the identification of macromolecule variants with specific or enhanced characteristics.

Random mutagenesis has been the most widely recognized method to date. Typically, this has been carried out either through the use of “error-prone” PCR (as described in Moore, J., et al, Nature Biotechnology 14:458, (1996), or through the application of randomized synthetic oligonucleotides corresponding to specific regions of interest (as described by Derbyshire, K. M. et al, Gene, 46:145-152, (1986), and Hill, D E, et al, Methods Enzymol., 55:559-568, (1987). Both approaches have limits to the level of mutagenesis that can be obtained. However, either approach enables the investigator to effectively control the rate of mutagenesis. This is particularly important considering the fact that mutations beneficial to the activity of the enzyme are fairly rare. In fact, using too high a level of mutagenesis may counter or inhibit the desired benefit of a useful mutation.

While both of the aforementioned methods are effective for creating randomized pools of macromolecule variants, a third method, termed “DNA Shuffling”, or “sexual PCR” (WPC, Stemmer, PNAS, 91:10747, (1994)) has recently been elucidated. DNA shuffling has also been referred to as “directed molecular evolution”, “exon-shuffling”, “directed enzyme evolution”, “in vitro evolution”, and “artificial evolution”. Such reference terms are known in the art and are encompassed by the invention. This new, preferred, method apparently overcomes the limitations of the previous methods in that it not only propagates positive traits, but simultaneously eliminates negative traits in the resulting progeny.

DNA shuffling accomplishes this task by combining the principal of in vitro recombination, along with the method of “error-prone” PCR. In effect, you begin with a randomly digested pool of small fragments of your gene, created by Dnase I digestion, and then introduce said random fragments into an “error-prone” PCR assembly reaction. During the PCR reaction, the randomly sized DNA fragments not only hybridize to their cognate strand, but also may hybridize to other DNA fragments corresponding to different regions of the polynucleotide of interest—regions not typically accessible via hybridization of the entire polynucleotide. Moreover, since the PCR assembly reaction utilizes “error-prone” PCR reaction conditions, random mutations are introduced during the DNA synthesis step of the PCR-reaction for all of the fragments—further diversifying the potential hybridization sites during the annealing step of the reaction.

A variety of reaction conditions could be utilized to carry out the DNA shuffling reaction. However, specific reaction conditions for DNA shuffling are provided, for example, in PNAS, 91:10747, (1994). Briefly:

Prepare the DNA substrate to be subjected to the DNA shuffling reaction. Preparation may be in the form of simply purifying the DNA from contaminating cellular material, chemicals, buffers, oligonucleotide primers, deoxynucleotides, RNAs, etc., and may entail the use of DNA purification kits as those provided by Qiagen, Inc., or by the Promega, Corp., for example.

Once the DNA substrate has been purified, it would be subjected to Dnase I digestion. About 2-4 ug of the DNA substrate(s) would be digested with 0.0015 units of Dnase I (Sigma) per ul in 100 ul of 50 mM Tris-HCL, pH 7.4/1 mM MgCl2 for 10-20 min. at room temperature. The resulting fragments of 10-50 bp could then be purified by running them through a 2% low-melting point agarose gel by electrophoresis onto DE81 ion-exchange paper (Whatmann) or could be purified using Microcon concentrators (Amicon) of the appropriate molecular weight cutoff, or could use oligonucleotide purification columns (Qiagen), in addition to other methods known in the art. If using DE81 ion-exchange paper, the 10-50 bp fragments could be eluted from said paper using 1 M NaCl, followed by ethanol precipitation.

The resulting purified fragments would then be subjected to a PCR assembly reaction by re-suspension in a PCR mixture containing: 2 mM of each dNTP, 2.2 mM MgCl2, 50 mM KCl, 10 mM Tris.HCL, pH 9.0, and 0.1% Triton X-100, at a final fragment concentration of 10-30 ng/ul. No primers are added at this point. Taq DNA polymerase (Promega) would be used at 2.5 units per 100 ul of reaction mixture. A PCR program of 94 C for 60s; 94 C for 30s, 50-55 C for 30s, and 72 C for 30s using 30-45 cycles, followed by 72 C for 5 min using an MJ Research (Cambridge, Mass.) PTC-150 thermocycler. After the assembly reaction is completed, a 1:40 dilution of the resulting primerless product would then be introduced into a PCR mixture (using the same buffer mixture used for the assembly reaction) containing 0.8 um of each primer and subjecting this mixture to 15 cycles of PCR (using 94 C for 30s, 50 C for 30s, and 72 C for 30s). The referred primers would be primers corresponding to the nucleic acid sequences of the polynucleotide(s) utilized in the shuffling reaction. Said primers could consist of modified nucleic acid base pairs using methods known in the art and referred to else where herein, or could contain additional sequences (i.e., for adding restriction sites, mutating specific base-pairs, etc.).

The resulting shuffled, assembled, and amplified product can be purified using methods well known in the art (e.g., Qiagen PCR purification kits) and then subsequently cloned using appropriate restriction enzymes.

Although a number of variations of DNA shuffling have been published to date, such variations would be obvious to the skilled artisan and are encompassed by the invention. The DNA shuffling method can also be tailored to the desired level of mutagenesis using the methods described by Zhao, et al. (Nucl Acid Res., 25(6): 1307-1308, (1997).

As described above, once the randomized pool has been created, it can then be subjected to a specific screen to identify the variant possessing the desired characteristic(s). Once the variant has been identified, DNA corresponding to the variant could then be used as the DNA substrate for initiating another round of DNA shuffling. This cycle of shuffling, selecting the optimized variant of interest, and then re-shuffling, can be repeated until the ultimate variant is obtained. Examples of model screens applied to identify variants created using DNA shuffling technology may be found in the following publications: J. C., Moore, et al., J. Mol. Biol., 272:336-347, (1997), F. R., Cross, et al., Mol. Cell. Biol., 18:2923-2931, (1998), and A. Crameri., et al., Nat. Biotech., 15:436-438, (1997).

DNA shuffling has several advantages. First, it makes use of beneficial mutations. When combined with screening, DNA shuffling allows the discovery of the best mutational combinations and does not assume that the best combination contains all the mutations in a population. Secondly, recombination occurs simultaneously with point mutagenesis. An effect of forcing DNA polymerase to synthesize full-length genes from the small fragment DNA pool is a background mutagenesis rate. In combination with a stringent selection method, enzymatic activity has been evolved up to 16000 fold increase over the wild-type form of the enzyme. In essence, the background mutagenesis yielded the genetic variability on which recombination acted to enhance the activity.

A third feature of recombination is that it can be used to remove deleterious mutations. As discussed above, during the process of the randomization, for every one beneficial mutation, there may be at least one or more neutral or inhibitory mutations. Such mutations can be removed by including in the assembly reaction an excess of the wild-type random-size fragments, in addition to the random-size fragments of the selected mutant from the previous selection. During the next selection, some of the most active variants of the polynucleotide/polypeptide/enzyme, should have lost the inhibitory mutations.

Finally, recombination enables parallel processing. This represents a significant advantage since there are likely multiple characteristics that would make a protein more desirable (e.g. solubility, activity, etc.). Since it is increasingly difficult to screen for more than one desirable trait at a time, other methods of molecular evolution tend to be inhibitory. However, using recombination, it would be possible to combine the randomized fragments of the best representative variants for the various traits, and then select for multiple properties at once.

DNA shuffling can also be applied to the polynucleotides and polypeptides of the present invention to decrease their immunogenicity in a specified host, particularly if the polynucleotides and polypeptides provide a therapeutic use. For example, a particular variant of the present invention may be created and isolated using DNA shuffling technology. Such a variant may have all of the desired characteristics, though may be highly immunogenic in a host due to its novel intrinsic structure. Specifically, the desired characteristic may cause the polypeptide to have a non-native structure which could no longer be recognized as a “self” molecule, but rather as a “foreign”, and thus activate a host immune response directed against the novel variant. Such a limitation can be overcome, for example, by including a copy of the gene sequence for a xenobiotic ortholog of the native protein in with the gene sequence of the novel variant gene in one or more cycles of DNA shuffling. The molar ratio of the ortholog and novel variant DNAs could be varied accordingly. Ideally, the resulting hybrid variant identified would contain at least some of the coding sequence which enabled the xenobiotic protein to evade the host immune system, and additionally, the coding sequence of the original novel variant that provided the desired characteristics.

Likewise, the invention encompasses the application of DNA shuffling technology to the evolution of polynucleotides and polypeptides of the invention, wherein one or more cycles of DNA shuffling include, in addition to the gene template DNA, oligonucleotides coding for known allelic sequences, optimized codon sequences, known variant sequences, known polynucleotide polymorphism sequences, known ortholog sequences, known homologue sequences, additional homologous sequences, additional non-homologous sequences, sequences from another species, and any number and combination of the above.

In addition to the described methods above, there are a number of related methods that may also be applicable, or desirable in certain cases. Representative among these are the methods discussed in PCT applications WO 98/31700, and WO 98/32845, which are hereby incorporated by reference. Furthermore, related methods can also be applied to the polynucleotide sequences of the present invention in order to evolve invention for creating ideal variants for use in gene therapy, protein engineering, evolution of whole cells containing the variant, or in the evolution of entire enzyme pathways containing polynucleotides of the invention as described in PCT applications WO 98/13485, WO 98/13487, WO 98/27230, WO 98/31837, and Crameri, A., et al., Nat. Biotech., 15:436-438, (1997), respectively.

Additional methods of applying “DNA Shuffling” technology to the polynucleotides and polypeptides of the present invention, including their proposed applications, may be found in U.S. Pat. No. 5,605,793; PCT Application No. WO 95/22625; PCT Application No. WO 97/20078; PCT Application No. WO 97/35966; and PCT Application No. WO 98/42832; PCT Application No. The forgoing are hereby incorporated in their entirety herein for all purposes.

### Example 20

### Gene Therapy Using Endogenous Genes Corresponding to Polynucleotides of the Invention

Another method of gene therapy according to the present invention involves operably associating the endogenous polynucleotide sequence of the invention with a promoter via homologous recombination as described, for example, in U.S. Pat. No. 5,641,670, issued Jun. 24, 1997; International Publication NO:WO 96/29411, published Sep. 26, 1996; International Publication NO:WO 94/12650, published Aug. 4, 1994; Koller et al., Proc. Natl. Acad. Sci. USA, 86:8932-8935 (1989); and Zijlstra et al., Nature, 342:435-438 (1989). This method involves the activation of a gene which is present in the target cells, but which is not expressed in the cells, or is expressed at a lower level than desired.

Polynucleotide constructs are made which contain a promoter and targeting sequences, which are homologous to the 5′ non-coding sequence of endogenous polynucleotide sequence, flanking the promoter. The targeting sequence will be sufficiently near the 5′ end of the polynucleotide sequence so the promoter will be operably linked to the endogenous sequence upon homologous recombination. The promoter and the targeting sequences can be amplified using PCR. Preferably, the amplified promoter contains distinct restriction enzyme sites on the 5′ and 3′ ends. Preferably, the 3′ end of the first targeting sequence contains the same restriction enzyme site as the 5′ end of the amplified promoter and the 5′ end of the second targeting sequence contains the same restriction site as the 3′ end of the amplified promoter.

The amplified promoter and the amplified targeting sequences are digested with the appropriate restriction enzymes and subsequently treated with calf intestinal phosphatase. The digested promoter and digested targeting sequences are added together in the presence of T4 DNA ligase. The resulting mixture is maintained under conditions appropriate for ligation of the two fragments. The construct is size fractionated on an agarose gel then purified by phenol extraction and ethanol precipitation.

In this Example, the polynucleotide constructs are administered as naked polynucleotides via electroporation. However, the polynucleotide constructs may also be administered with transfection-facilitating agents, such as liposomes, viral sequences, viral particles, precipitating agents, etc. Such methods of delivery are known in the art.

Once the cells are transfected, homologous recombination will take place which results in the promoter being operably linked to the endogenous polynucleotide sequence. This results in the expression of polynucleotide corresponding to the polynucleotide in the cell. Expression may be detected by immunological staining, or any other method known in the art.

Fibroblasts are obtained from a subject by skin biopsy. The resulting tissue is placed in DMEM+10% fetal calf serum. Exponentially growing or early stationary 10 phase fibroblasts are trypsinized and rinsed from the plastic surface with nutrient medium. An aliquot of the cell suspension is removed for counting, and the remaining cells are subjected to centrifugation. The supernatant is aspirated and the pellet is resuspended in 5 ml of electroporation buffer (20 mM HEPES pH 7.3, 137 mM NaCl, 5 mM KCl, 0.7 mM Na2 HPO4, 6 mM dextrose). The cells are recentrifuged, the supernatant aspirated, and the cells resuspended in electroporation buffer containing 1 mg/ml acetylated bovine serum albumin. The final cell suspension contains approximately 3×10⁶ cells/ml. Electroporation should be performed immediately following resuspension.

Plasmid DNA is prepared according to standard techniques. For example, to construct a plasmid for targeting to the locus corresponding to the polynucleotide of the invention, plasmid pUC18 (MBI Fermentas, Amherst, N.Y.) is digested with HindIII. The CMV promoter is amplified by PCR with an XbaI site on the 5′ end and a BamHI site on the 3′end. Two non-coding sequences are amplified via PCR: one non-coding sequence (fragment 1) is amplified with a HindIII site at the 5′ end and an Xba site at the 3′end; the other non-coding sequence (fragment 2) is amplified with a BamHI site at the 5′end and a HindIII site at the 3′end. The CMV promoter and the fragments (1 and 2) are digested with the appropriate enzymes (CMV promoter—XbaI and BamHI; fragment 1—XbaI; fragment 2—BamHI) and ligated together. The resulting ligation product is digested with HindIII, and ligated with the HindIII-digested pUC18 plasmid.

Plasmid DNA is added to a sterile cuvette with a 0.4 cm electrode gap (Bio-Rad). The final DNA concentration is generally at least 120 μg/ml. 0.5 ml of the cell suspension (containing approximately 1.5.×106 cells) is then added to the cuvette, and the cell suspension and DNA solutions are gently mixed. Electroporation is performed with a Gene-Pulser apparatus (Bio-Rad). Capacitance and voltage are set at 960 μF and 250-300 V, respectively. As voltage increases, cell survival decreases, but the percentage of surviving cells that stably incorporate the introduced DNA into their genome increases dramatically. Given these parameters, a pulse time of approximately 14-20 mSec should be observed.

Electroporated cells are maintained at room temperature for approximately 5 min, and the contents of the cuvette are then gently removed with a sterile transfer pipette. The cells are added directly to 10 ml of prewarmed nutrient media (DMEM with 15% calf serum) in a 10 cm dish and incubated at 37 degree C. The following day, the media is aspirated and replaced with 10 ml of fresh media and incubated for a further 16-24 hours.

The engineered fibroblasts are then injected into the host, either alone or after having been grown to confluence on cytodex 3 microcarrier beads. The fibroblasts now produce the protein product. The fibroblasts can then be introduced into a patient as described above.

### Example 21

### Method of Treatment Using Gene Therapy—In Vivo

Another aspect of the present invention is using in vivo gene therapy methods to treat disorders, diseases and conditions. The gene therapy method relates to the introduction of naked nucleic acid (DNA, RNA, and antisense DNA or RNA) sequences into an animal to increase or decrease the expression of the polypeptide. The polynucleotide of the present invention may be operatively linked to a promoter or any other genetic elements necessary for the expression of the polypeptide by the target tissue. Such gene therapy and delivery techniques and methods are known in the art, see, for example, WO90/11092, WO98/11779; U.S. Pat. No. 5,693,622, 5,705,151, 5,580,859; Tabata et al., Cardiovasc. Res. 35(3):470-479 (1997); Chao et al., Pharmacol. Res. 35(6):517-522 (1997); Wolff, Neuromuscul. Disord. 7(5):314-318 (1997); Schwartz et al., Gene Ther. 3(5):405-411 (1996); Tsurumi et al., Circulation 94(12):3281-3290 (1996) (incorporated herein by reference).

The polynucleotide constructs may be delivered by any method that delivers injectable materials to the cells of an animal, such as, injection into the interstitial space of tissues (heart, muscle, skin, lung, liver, intestine and the like). The polynucleotide constructs can be delivered in a pharmaceutically acceptable liquid or aqueous carrier.

The term “naked” polynucleotide, DNA or RNA, refers to sequences that are free from any delivery vehicle that acts to assist, promote, or facilitate entry into the cell, including viral sequences, viral particles, liposome formulations, lipofectin or precipitating agents and the like. However, the polynucleotides of the present invention may also be delivered in liposome formulations (such as those taught in Feigner P. L. et al. (1995) Ann. NY Acad. Sci. 772:126-139 and Abdallah B. et al. (1995) Biol. Cell 85(1):1-7) which can be prepared by methods well known to those skilled in the art.

The polynucleotide vector constructs used in the gene therapy method are preferably constructs that will not integrate into the host genome nor will they contain sequences that allow for replication. Any strong promoter known to those skilled in the art can be used for driving the expression of DNA. Unlike other gene therapies techniques, one major advantage of introducing naked nucleic acid sequences into target cells is the transitory nature of the polynucleotide synthesis in the cells. Studies have shown that non-replicating DNA sequences can be introduced into cells to provide production of the desired polypeptide for periods of up to six months.

The polynucleotide construct can be delivered to the interstitial space of tissues within the an animal, including of muscle, skin, brain, lung, liver, spleen, bone marrow, thymus, heart, lymph, blood, bone, cartilage, pancreas, kidney, gall bladder, stomach, intestine, testis, ovary, uterus, rectum, nervous system, eye, gland, and connective tissue. Interstitial space of the tissues comprises the intercellular fluid, mucopolysaccharide matrix among the reticular fibers of organ tissues, elastic fibers in the walls of vessels or chambers, collagen fibers of fibrous tissues, or that same matrix within connective tissue ensheathing muscle cells or in the lacunae of bone. It is similarly the space occupied by the plasma of the circulation and the lymph fluid of the lymphatic channels. Delivery to the interstitial space of muscle tissue is preferred for the reasons discussed below. They may be conveniently delivered by injection into the tissues comprising these cells. They are preferably delivered to and expressed in persistent, non-dividing cells which are differentiated, although delivery and expression may be achieved in non-differentiated or less completely differentiated cells, such as, for example, stem cells of blood or skin fibroblasts. In vivo muscle cells are particularly competent in their ability to take up and express polynucleotides.

For the naked polynucleotide injection, an effective dosage amount of DNA or RNA will be in the range of from about 0.05 g/kg body weight to about 50 mg/kg body weight. Preferably the dosage will be from about 0.005 mg/kg to about 20 mg/kg and more preferably from about 0.05 mg/kg to about 5 mg/kg. Of course, as the artisan of ordinary skill will appreciate, this dosage will vary according to the tissue site of injection. The appropriate and effective dosage of nucleic acid sequence can readily be determined by those of ordinary skill in the art and may depend on the condition being treated and the route of administration. The preferred route of administration is by the parenteral route of injection into the interstitial space of tissues. However, other parenteral routes may also be used, such as, inhalation of an aerosol formulation particularly for delivery to lungs or bronchial tissues, throat or mucous membranes of the nose. In addition, naked polynucleotide constructs can be delivered to arteries during angioplasty by the catheter used in the procedure.

The dose response effects of injected polynucleotide in muscle in vivo is determined as follows. Suitable template DNA for production of mRNA coding for polypeptide of the present invention is prepared in accordance with a standard recombinant DNA methodology. The template DNA, which may be either circular or linear, is either used as naked DNA or complexed with liposomes. The quadriceps muscles of mice are then injected with various amounts of the template DNA.

Five to six week old female and male Balb/C mice are anesthetized by intraperitoneal injection with 0.3 ml of 2.5% Avertin. A 1.5 cm incision is made on the anterior thigh, and the quadriceps muscle is directly visualized. The template DNA is injected in 0.1 ml of carrier in a 1 cc syringe through a 27 gauge needle over one minute, approximately 0.5 cm from the distal insertion site of the muscle into the knee and about 0.2 cm deep. A suture is placed over the injection site for future localization, and the skin is closed with stainless steel clips.

After an appropriate incubation time (e.g., 7 days) muscle extracts are prepared by excising the entire quadriceps. Every fifth 15 um cross-section of the individual quadriceps muscles is histochemically stained for protein expression. A time course for protein expression may be done in a similar fashion except that quadriceps from different mice are harvested at different times. Persistence of DNA in muscle following injection may be determined by Southern blot analysis after preparing total cellular DNA and HIRT supernatants from injected and control mice. The results of the above experimentation in mice can be use to extrapolate proper dosages and other treatment parameters in humans and other animals using naked DNA.

### Example 22

### Transgenic Animals

The polypeptides of the invention can also be expressed in transgenic animals. Animals of any species, including, but not limited to, mice, rats, rabbits, hamsters, guinea pigs, pigs, micro-pigs, goats, sheep, cows and non-human primates, e.g., baboons, monkeys, and chimpanzees may be used to generate transgenic animals. In a specific embodiment, techniques described herein or otherwise known in the art, are used to express polypeptides of the invention in humans, as part of a gene therapy protocol.

Any technique known in the art may be used to introduce the transgene (i.e., polynucleotides of the invention) into animals to produce the founder lines of transgenic animals. Such techniques include, but are not limited to, pronuclear microinjection (Paterson et al., Appl. Microbiol. Biotechnol. 40:691-698 (1994); Carver et al., Biotechnology (NY) 11:1263-1270 (1993); Wright et al., Biotechnology (NY) 9:830-834 (1991); and Hoppe et al., U.S. Pat. No. 4,873,191 (1989)); retrovirus mediated gene transfer into germ lines (Van der Putten et al., Proc. Natl. Acad. Sci., USA 82:6148-6152 (1985)), blastocysts or embryos; gene targeting in embryonic stem cells (Thompson et al., Cell 56:313-321 (1989)); electroporation of cells or embryos (Lo, 1983, Mol Cell. Biol. 3:1803-1814 (1983)); introduction of the polynucleotides of the invention using a gene gun (see, e.g., Ulmer et al., Science 259:1745 (1993); introducing nucleic acid constructs into embryonic pleuripotent stem cells and transferring the stem cells back into the blastocyst; and sperm-mediated gene transfer (Lavitrano et al., Cell 57:717-723 (1989); etc. For a review of such techniques, see Gordon, “Transgenic Animals” Intl. Rev. Cytol. 115:171-229 (1989), which is incorporated by reference herein in its entirety.

Any technique known in the art may be used to produce transgenic clones containing polynucleotides of the invention, for example, nuclear transfer into enucleated oocytes of nuclei from cultured embryonic, fetal, or adult cells induced to quiescence (Campell et al., Nature 380:64-66 (1996); Wilmut et al., Nature 385:810-813 (1997)).

The present invention provides for transgenic animals that carry the transgene in all their cells, as well as animals which carry the transgene in some, but not all their cells, i.e., mosaic animals or chimeric. The transgene may be integrated as a single transgene or as multiple copies such as in concatamers, e.g., head-to-head tandems or head-to-tail tandems. The transgene may also be selectively introduced into and activated in a particular cell type by following, for example, the teaching of Lasko et al. (Lasko et al., Proc. Natl. Acad. Sci. USA 89:6232-6236 (1992)). The regulatory sequences required for such a cell-type specific activation will depend upon the particular cell type of interest, and will be apparent to those of skill in the art. When it is desired that the polynucleotide transgene be integrated into the chromosomal site of the endogenous gene, gene targeting is preferred. Briefly, when such a technique is to be utilized, vectors containing some nucleotide sequences homologous to the endogenous gene are designed for the purpose of integrating, via homologous recombination with chromosomal sequences, into and disrupting the function of the nucleotide sequence of the endogenous gene. The transgene may also be selectively introduced into a particular cell type, thus inactivating the endogenous gene in only that cell type, by following, for example, the teaching of Gu et al. (Gu et al., Science 265:103-106 (1994)). The regulatory sequences required for such a cell-type specific inactivation will depend upon the particular cell type of interest, and will be apparent to those of skill in the art.

Once transgenic animals have been generated, the expression of the recombinant gene may be assayed utilizing standard techniques. Initial screening may be accomplished by Southern blot analysis or PCR techniques to analyze animal tissues to verify that integration of the transgene has taken place. The level of mRNA expression of the transgene in the tissues of the transgenic animals may also be assessed using techniques which include, but are not limited to, Northern blot analysis of tissue samples obtained from the animal, in situ hybridization analysis, and reverse transcriptase-PCR(RT-PCR). Samples of transgenic gene-expressing tissue may also be evaluated immunocytochemically or immunohistochemically using antibodies specific for the transgene product.

Once the founder animals are produced, they may be bred, inbred, outbred, or crossbred to produce colonies of the particular animal. Examples of such breeding strategies include, but are not limited to: outbreeding of founder animals with more than one integration site in order to establish separate lines; inbreeding of separate lines in order to produce compound transgenics that express the transgene at higher levels because of the effects of additive expression of each transgene; crossing of heterozygous transgenic animals to produce animals homozygous for a given integration site in order to both augment expression and eliminate the need for screening of animals by DNA analysis; crossing of separate homozygous lines to produce compound heterozygous or homozygous lines; and breeding to place the transgene on a distinct background that is appropriate for an experimental model of interest.

Transgenic animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 23

### Knock-Out Animals

Endogenous gene expression can also be reduced by inactivating or “knocking out” the gene and/or its promoter using targeted homologous recombination. (E.g., see Smithies et al., Nature 317:230-234 (1985); Thomas & Capecchi, Cell 51:503-512 (1987); Thompson et al., Cell 5:313-321 (1989); each of which is incorporated by reference herein in its entirety). For example, a mutant, non-functional polynucleotide of the invention (or a completely unrelated DNA sequence) flanked by DNA homologous to the endogenous polynucleotide sequence (either the coding regions or regulatory regions of the gene) can be used, with or without a selectable marker and/or a negative selectable marker, to transfect cells that express polypeptides of the invention in vivo. In another embodiment, techniques known in the art are used to generate knockouts in cells that contain, but do not express the gene of interest. Insertion of the DNA construct, via targeted homologous recombination, results in inactivation of the targeted gene. Such approaches are particularly suited in research and agricultural fields where modifications to embryonic stem cells can be used to generate animal offspring with an inactive targeted gene (e.g., see Thomas & Capecchi 1987 and Thompson 1989, supra). However this approach can be routinely adapted for use in humans provided the recombinant DNA constructs are directly administered or targeted to the required site in vivo using appropriate viral vectors that will be apparent to those of skill in the art.

In further embodiments of the invention, cells that are genetically engineered to express the polypeptides of the invention, or alternatively, that are genetically engineered not to express the polypeptides of the invention (e.g., knockouts) are administered to a patient in vivo. Such cells may be obtained from the patient (i.e., animal, including human) or an MHC compatible donor and can include, but are not limited to fibroblasts, bone marrow cells, blood cells (e.g., lymphocytes), adipocytes, muscle cells, endothelial cells etc. The cells are genetically engineered in vitro using recombinant DNA techniques to introduce the coding sequence of polypeptides of the invention into the cells, or alternatively, to disrupt the coding sequence and/or endogenous regulatory sequence associated with the polypeptides of the invention, e.g., by transduction (using viral vectors, and preferably vectors that integrate the transgene into the cell genome) or transfection procedures, including, but not limited to, the use of plasmids, cosmids, YACs, naked DNA, electroporation, liposomes, etc. The coding sequence of the polypeptides of the invention can be placed under the control of a strong constitutive or inducible promoter or promoter/enhancer to achieve expression, and preferably secretion, of the polypeptides of the invention. The engineered cells which express and preferably secrete the polypeptides of the invention can be introduced into the patient systemically, e.g., in the circulation, or intraperitoneally.

Alternatively, the cells can be incorporated into a matrix and implanted in the body, e.g., genetically engineered fibroblasts can be implanted as part of a skin graft; genetically engineered endothelial cells can be implanted as part of a lymphatic or vascular graft. (See, for example, Anderson et al. U.S. Pat. No. 5,399,349; and Mulligan & Wilson, U.S. Pat. No. 5,460,959 each of which is incorporated by reference herein in its entirety).

When the cells to be administered are non-autologous or non-MHC compatible cells, they can be administered using well known techniques which prevent the development of a host immune response against the introduced cells. For example, the cells may be introduced in an encapsulated form which, while allowing for an exchange of components with the immediate extracellular environment, does not allow the introduced cells to be recognized by the host immune system.

Transgenic and “knock-out” animals of the invention have uses which include, but are not limited to, animal model systems useful in elaborating the biological function of polypeptides of the present invention, studying diseases, disorders, and/or conditions associated with aberrant expression, and in screening for compounds effective in ameliorating such diseases, disorders, and/or conditions.

### Example 24

### Biological Effects of BMSOTR and/or BMSOTR Variant Polypeptides of the Invention Astrocyte and Neuronal Assays

Recombinant polypeptides of the invention, expressed in Escherichia coli and purified as described above, can be tested for activity in promoting the survival, neurite outgrowth, or phenotypic differentiation of cortical neuronal cells and for inducing the proliferation of glial fibrillary acidic protein immunopositive cells, astrocytes. The selection of cortical cells for the bioassay is based on the prevalent expression of FGF-1 and FGF-2 in cortical structures and on the previously reported enhancement of cortical neuronal survival resulting from FGF-2 treatment. A thymidine incorporation assay, for example, can be used to elucidate a polypeptide of the invention's activity on these cells.

Moreover, previous reports describing the biological effects of FGF-2 (basic FGF) on cortical or hippocampal neurons in vitro have demonstrated increases in both neuron survival and neurite outgrowth (Walicke et al., “Fibroblast growth factor promotes survival of dissociated hippocampal neurons and enhances neurite extension.” Proc. Natl. Acad. Sci. USA 83:3012-3016. (1986), assay herein incorporated by reference in its entirety). However, reports from experiments done on PC-12 cells suggest that these two responses are not necessarily synonymous and may depend on not only which FGF is being tested but also on which receptor(s) are expressed on the target cells. Using the primary cortical neuronal culture paradigm, the ability of a polypeptide of the invention to induce neurite outgrowth can be compared to the response achieved with FGF-2 using, for example, a thymidine incorporation assay.

### Fibroblast and Endothelial Cell Assays

Human lung fibroblasts are obtained from Clonetics (San Diego, Calif.) and maintained in growth media from Clonetics. Dermal microvascular endothelial cells are obtained from Cell Applications (San Diego, Calif.). For proliferation assays, the human lung fibroblasts and dermal microvascular endothelial cells can be cultured at 5,000 cells/well in a 96-well plate for one day in growth medium. The cells are then incubated for one day in 0.1% BSA basal medium. After replacing the medium with fresh 0.1% BSA medium, the cells are incubated with the test proteins for 3 days. Alamar Blue (Alamar Biosciences, Sacramento, Calif.) is added to each well to a final concentration of 10%. The cells are incubated for 4 hr. Cell viability is measured by reading in a CytoFluor fluorescence reader. For the PGE2 assays, the human lung fibroblasts are cultured at 5,000 cells/well in a 96-well plate for one day. After a medium change to 0.1% BSA basal medium, the cells are incubated with FGF-2 or polypeptides of the invention with or without IL-1(for 24 hours. The supernatants are collected and assayed for PGE2 by EIA kit (Cayman, Ann Arbor, Mich.). For the IL-6 assays, the human lung fibroblasts are cultured at 5,000 cells/well in a 96-well plate for one day. After a medium change to 0.1% BSA basal medium, the cells are incubated with FGF-2 or with or without polypeptides of the invention IL-1 (for 24 hours. The supernatants are collected and assayed for IL-6 by ELISA kit (Endogen, Cambridge, Mass.).

Human lung fibroblasts are cultured with FGF-2 or polypeptides of the invention for 3 days in basal medium before the addition of Alamar Blue to assess effects on growth of the fibroblasts. FGF-2 should show a stimulation at 10-2500 ng/ml which can be used to compare stimulation with polypeptides of the invention.

### Parkinson Models

The loss of motor function in Parkinson's disease is attributed to a deficiency of striatal dopamine resulting from the degeneration of the nigrostriatal dopaminergic projection neurons. An animal model for Parkinson's that has been extensively characterized involves the systemic administration of 1-methyl-4 phenyl 1,2,3,6-tetrahydropyridine (MPTP). In the CNS, MPTP is taken-up by astrocytes and catabolized by monoamine oxidase B to 1-methyl-4-phenyl pyridine (MPP+) and released. Subsequently, MPP+is actively accumulated in dopaminergic neurons by the high-affinity reuptake transporter for dopamine. MPP+is then concentrated in mitochondria by the electrochemical gradient and selectively inhibits nicotidamide adenine disphosphate: ubiquinone oxidoreductionase (complex I), thereby interfering with electron transport and eventually generating oxygen radicals.

It has been demonstrated in tissue culture paradigms that FGF-2 (basic FGF) has trophic activity towards nigral dopaminergic neurons (Ferrari et al., Dev. Biol. 1989). Recently, Dr. Unsicker's group has demonstrated that administering FGF-2 in gel foam implants in the striatum results in the near complete protection of nigral dopaminergic neurons from the toxicity associated with MPTP exposure (Otto and Unsicker, J. Neuroscience, 1990).

Based on the data with FGF-2, polypeptides of the invention can be evaluated to determine whether it has an action similar to that of FGF-2 in enhancing dopaminergic neuronal survival in vitro and it can also be tested in vivo for protection of dopaminergic neurons in the striatum from the damage associated with MPTP treatment. The potential effect of a polypeptide of the invention is first examined in vitro in a dopaminergic neuronal cell culture paradigm. The cultures are prepared by dissecting the midbrain floor plate from gestation day 14 Wistar rat embryos. The tissue is dissociated with trypsin and seeded at a density of 200,000 cells/cm2 on polyorthinine-laminin coated glass coverslips. The cells are maintained in Dulbecco's Modified Eagle's medium and F12 medium containing hormonal supplements (N1). The cultures are fixed with paraformaldehyde after 8 days in vitro and are processed for tyrosine hydroxylase, a specific marker for dopaminergic neurons, immunohistochemical staining. Dissociated cell cultures are prepared from embryonic rats. The culture medium is changed every third day and the factors are also added at that time.

Since the dopaminergic neurons are isolated from animals at gestation day 14, a developmental time which is past the stage when the dopaminergic precursor cells are proliferating, an increase in the number of tyrosine hydroxylase immunopositive neurons would represent an increase in the number of dopaminergic neurons surviving in vitro. Therefore, if a polypeptide of the invention acts to prolong the survival of dopaminergic neurons, it would suggest that the polypeptide may be involved in Parkinson's Disease.

One skilled in the art could easily modify the exemplified studies to test the activity of polynucleotides of the invention (e.g., gene therapy), agonists, and/or antagonists of polynucleotides or polypeptides of the invention.

The entire disclosure of each document cited (including patents, patent applications, journal articles, abstracts, laboratory manuals, books, or other disclosures) in the Background of the Invention, Detailed Description, and Examples is hereby incorporated herein by reference. Further, the hard copy of the sequence listing submitted herewith and the corresponding computer readable form are both incorporated herein by reference in their entireties.

As various changes can be made in the above-described subject matter without departing from the scope and spirit of the present invention, it is intended that all subject matter contained in the above description, or defined in the appended claims, be interpreted as descriptive and illustrative of the present invention. Many modifications and variations of the present invention are possible in light of the above teachings.

What is claimed is:

1. An isolated nucleic acid molecule comprising a polynucleotide having a nucleotide sequence selected from the group consisting of:
(a) a polynucleotide fragment of SEQ ID NO:1 or a polynucleotide fragment of the cDNA sequence included in ATCC Deposit No: ______, which is hybridizable to SEQ ID NO:1;
(b) a polynucleotide encoding a polypeptide fragment of SEQ ID NO:2 or a polypeptide fragment encoded by the cDNA sequence included in ATCC Deposit No: ______, which is hybridizable to SEQ ID NO:1;
(c) a polynucleotide encoding a polypeptide domain of SEQ ID NO:2 or a polypeptide domain encoded by the cDNA sequence included in ATCC Deposit No: ______, which is hybridizable to SEQ ID NO:1;
(d) a polynucleotide encoding a polypeptide epitope of SEQ ID NO:2 or a polypeptide epitope encoded by the cDNA sequence included in ATCC Deposit No: ______, which is hybridizable to SEQ ID NO:1;
(e) a polynucleotide encoding a polypeptide of SEQ ID NO:2 or the cDNA sequence included in ATCC Deposit No: ______, which is hybridizable to SEQ ID NO:1, having GPCR activity;
(f) an isolated polynucleotide comprising nucleotides 119 to 1264 of SEQ ID NO:1, wherein said nucleotides encode a polypeptide corresponding to amino acids 2 to 383 of SEQ ID NO:2 minus the start codon;
(g) an isolated polynucleotide comprising nucleotides 116 to 1264 of SEQ ID NO:1, wherein said nucleotides encode a polypeptide corresponding to amino acids 1 to 383 of SEQ ID NO:2 including the start codon;
(h) a polynucleotide which represents the complimentary sequence (antisense) of SEQ ID NO:1;
(i) a polynucleotide fragment of SEQ ID NO:3 or a polynucleotide fragment of the cDNA sequence included in ATCC Deposit No: ______, which is hybridizable to SEQ ID NO:3;
(j) a polynucleotide encoding a polypeptide fragment of SEQ ID NO:4 or a polypeptide fragment encoded by the cDNA sequence included in ATCC Deposit No: ______, which is hybridizable to SEQ ID NO:3;
(k) a polynucleotide encoding a polypeptide domain of SEQ ID NO:4 or a polypeptide domain encoded by the cDNA sequence included in ATCC Deposit No: ______, which is hybridizable to SEQ ID NO:3;
(l) a polynucleotide encoding a polypeptide epitope of SEQ ID NO:4 or a polypeptide epitope encoded by the cDNA sequence included in ATCC Deposit No: ______, which is hybridizable to SEQ ID NO:3;
(m) a polynucleotide encoding a polypeptide of SEQ ID NO:4 or the cDNA sequence included in ATCC Deposit No: ______, which is hybridizable to SEQ ID NO:3, having GPCR activity;
(n) a polynucleotide encoding a polypeptide of SEQ ID NO:4, which is hybridizable to SEQ ID NO:3, having GPCR activity;
(o) an isolated polynucleotide comprising nucleotides 119 to 1417 of SEQ ID NO:3, wherein said nucleotides encode a polypeptide corresponding to amino acids 2 to 434 of SEQ ID NO:4 minus the start codon;
(p) an isolated polynucleotide comprising nucleotides 116 to 1417 of SEQ ID NO:3, wherein said nucleotides encode a polypeptide corresponding to amino acids 1 to 434 of SEQ ID NO:4 including the start codon;
(q) a polynucleotide which represents the complimentary sequence (antisense) of SEQ ID NO:3; and
(r) a polynucleotide capable of hybridizing under stringent conditions to any one of the polynucleotides specified in (a)-(1), wherein said polynucleotide does not hybridize under stringent conditions to a nucleic acid molecule having a nucleotide sequence of only A residues or of only T residues.

2. The isolated nucleic acid molecule of claim 1, wherein the polynucleotide fragment consists of a nucleotide sequence encoding a human G-protein coupled receptor.

3. A recombinant vector comprising the isolated nucleic acid molecule of claim 1.

4. A recombinant host cell comprising the vector sequences of claim 3.

5. An isolated polypeptide comprising an amino acid sequence selected from the group consisting of:
(a) a polypeptide fragment of SEQ ID NO:2 or the encoded sequence included in ATCC Deposit No: ______;
(b) a polypeptide fragment of SEQ ID NO:2 or the encoded sequence included in ATCC Deposit No: ______, having GPCR activity;
(c) a polypeptide domain of SEQ ID NO:2 or the encoded sequence included in ATCC Deposit No: ______;
(d) a polypeptide epitope of SEQ ID NO:2 or the encoded sequence included in ATCC Deposit No: ______;
(e) a full length protein of SEQ ID NO:2 or the encoded sequence included in ATCC Deposit No: ______;
(f) a polypeptide comprising amino acids 2 to 383 of SEQ ID NO:2, wherein said amino acids 2 to 383 comprising a polypeptide of SEQ ID NO:2 minus the start methionine;
(g) a polypeptide comprising amino acids 1 to 383 of SEQ ID NO:2;
(h) a polypeptide fragment of SEQ ID NO:4 or the encoded sequence included in ATCC Deposit No: ______;
(i) a polypeptide fragment of SEQ ID NO:4 or the encoded sequence included in ATCC Deposit No: ______, having GPCR activity;
(j) a polypeptide domain of SEQ ID NO:4 or the encoded sequence included in ATCC Deposit No: ______;
(k) a polypeptide epitope of SEQ ID NO:4 or the encoded sequence included in ATCC Deposit No: ______;
(l) a full length protein of SEQ ID NO:4 or the encoded sequence included in ATCC Deposit No: ______; a full length protein of SEQ ID NO:4;
(m) a polypeptide comprising amino acids 2 to 434 of SEQ ID NO:4, wherein said amino acids 2 to 434 comprising a polypeptide of SEQ ID NO:4 minus the start methionine; and
(n) a polypeptide comprising amino acids 1 to 434 of SEQ ID NO:4.

6. The isolated polypeptide of claim 5, wherein the full length protein comprises sequential amino acid deletions from either the C-terminus or the N-terminus.

7. An isolated antibody that binds specifically to the isolated polypeptide of claim 5.

8. A recombinant host cell that expresses the isolated polypeptide of claim 5.

9. A method of making an isolated polypeptide comprising:
(a) culturing the recombinant host cell of claim 8 under conditions such that said polypeptide is expressed; and
(b) recovering said polypeptide.

10. The polypeptide produced by claim 9.

11. A method for preventing, treating, or ameliorating a medical condition, comprising the step of administering to a mammalian subject a therapeutically effective amount of the polypeptide of claim 5, or a modulator thereof.

12. A method of diagnosing a pathological condition or a susceptibility to a pathological condition in a subject comprising:
(a) determining the presence or absence of a mutation in the polynucleotide of claim 1; and
(b) diagnosing a pathological condition or a susceptibility to a pathological condition based on the presence or absence of said mutation.

13. A method of diagnosing a pathological condition or a susceptibility to a pathological condition in a subject comprising:
(a) determining the presence or amount of expression of the polypeptide of claim 5 in a biological sample; and
(b) diagnosing a pathological condition or a susceptibility to a pathological condition based on the presence or amount of expression of the polypeptide.

14. An isolated nucleic acid molecule consisting of a polynucleotide having a nucleotide sequence selected from the group consisting of:
(a) a polynucleotide encoding a polypeptide of SEQ ID NO:2;
(b) an isolated polynucleotide consisting of nucleotides 119 to 1264 of SEQ ID NO:1, wherein said nucleotides encode a polypeptide corresponding to amino acids 2 to 383 of SEQ ID NO:2 minus the start codon;
(c) an isolated polynucleotide consisting of nucleotides 116 to 1264 of SEQ ID NO:1, wherein said nucleotides encode a polypeptide corresponding to amino acids 1 to 383 of SEQ ID NO:2 including the start codon;
(d) a polynucleotide encoding the HGPRBMY34 polypeptide encoded by the cDNA clone contained in ATCC Deposit No. ______;
(e) a polynucleotide which represents the complimentary sequence (antisense) of SEQ ID NO:1;
(f) a polynucleotide encoding a polypeptide of SEQ ID NO:4;
(g) an isolated polynucleotide consisting of nucleotides 119 to 1417 of SEQ ID NO:3, wherein said nucleotides encode a polypeptide corresponding to amino acids 2 to 434 of SEQ ID NO:4 minus the start codon;
(h) an isolated polynucleotide consisting of nucleotides 116 to 1417 of SEQ ID NO:3, wherein said nucleotides encode a polypeptide corresponding to amino acids 1 to 434 of SEQ ID NO:4 including the start codon;
(i) a polynucleotide encoding the HGPRBMY34 variant polypeptide encoded by the cDNA clone contained in ATCC Deposit No. ______; and
(j) a polynucleotide which represents the complimentary sequence (antisense) of SEQ ID NO:3.

15. The isolated nucleic acid molecule of claim 14, wherein the polynucleotide comprises a nucleotide sequence encoding a human G-protein coupled receptor.

16. A recombinant vector comprising the isolated nucleic acid molecule of claim 15.

17. A recombinant host cell comprising the recombinant vector of claim 16.

18. An isolated polypeptide consisting of an amino acid sequence selected from the group consisting of:
(a) a polypeptide fragment of SEQ ID NO:2 having GPCR activity;
(b) a polypeptide domain of SEQ ID NO:2 having GPCR activity;
(c) a full length protein of SEQ ID NO:2;
(d) a polypeptide corresponding to amino acids 2 to 383 of SEQ ID NO:2, wherein said amino acids 2 to 383 consisting of a polypeptide of SEQ ID NO:2 minus the start methionine;
(e) a polypeptide corresponding to amino acids 1 to 383 of SEQ ID NO:2;
(f) a polypeptide encoded by the cDNA contained in ATCC Deposit No. ______;
(g) a full length protein of SEQ ID NO:4;
(h) a polypeptide corresponding to amino acids 2 to 434 of SEQ ID NO:4, wherein said amino acids 2 to 434 consisting of a polypeptide of SEQ ID NO:4 minus the start methionine; and
(i) a polypeptide corresponding to amino acids 1 to 434 of SEQ ID NO:4.

19. The method of diagnosing a pathological condition of claim 15 wherein the condition is a member of the group consisting of: a disorder related to aberrant G-protein coupled signaling; oxytocin-related disorders; neurological disorders; a disorder related to aberrant cell cycle regulation; neurological disorders; anxiety; headache; migraine; manic depression; delirium; severe mental retardation; dyskinesias; neuropathic pain; altered behavior disorders; altered sexual behavior disorders; altered maternal behavior disorders; social behavior disorders; stress-related behavior disorders; feeding and grooming disorders; memory disorders; learning disorders; disorders involving altered ability to establish long term potentiation; sleep disorders; disorders associated with inability to establish or maintain circadian rhythms; balance disorders; perceptive disorders; anorexia; Prader-Willi syndrome; Alzheimer's disease; Parkinson's disease; Huntington's Disease; Tourette Syndrome; psychotic disorders; schizophrenia; mania; dementia; paranoia; depression; obsessive-compulsive disorders; panic disorder disorders involving altered synapse formation; disorders involving altered neurotransmission; demyelinating diseases; ALS; disorders involving altered cognition; disorders involving altered brain homeostasis; disorders involving altered neuronal differentiation or survival; disorders involving the maintenance of an attentive or alert state; disorders involving altered release or synthesis of neurotransmitters such as dopamine, opioid peptides, serotonin, GABA, and glutamate; addictive disorders; disorders involving altered tolerance to opioids; opioid addictions; meningitis; encephalitis; peripheral neuropathies; neoplasia; trauma; congenital malformations; spinal cord injuries; ischemia and infarction; aneurysms; hemorrhages; autism; heart and cardiovascular system disorders; acute heart failure; hypotension; hypertension; endocrinal diseases; growth disorders; obesity; anorexia; HIV infections; cancers; bulimia; asthma; osteoporosis; angina pectoris; myocardial infarction; reproductive disorders; female reproductive system disorders; male reproductive system disorders; mammary tissues disorders; immune-related disorders; kidney function; thymic involution; metabolic disorders; disorders of the pancreas; diabetes mellitus; diabetes; type 1 diabetes; type 2 diabetes; adult onset diabetes; indications related to islet cell transplantation; indications related to pancreatic transplantation; pancreatitis; pancreatic cancer; pancreatic exocrine insufficiency; alcohol induced pancreatitis; maldigestion of fat; maldigestion of protein; hypertriglyceridemia; vitamin b12 malabsorption; hypercalcemia; hypocalcemia; hyperglycemia; ascites; pleural effusions; abdominal pain; pancreatic necrosis; pancreatic abscess; pancreatic pseudocyst; gastrinomas; pancreatic islet cell hyperplasia; multiple endocrine neoplasia type 1 (men 1) syndrome; insulitis; amputations; diabetic neuropathy; pancreatic auto-immune disease; genetic defects of cell function; HNF-1 aberrations (formerly MODY3); glucokinase aberrations (formerly MODY2); HNF-4 aberrations (formerly MODY1); mitochondrial DNA aberrations; genetic defects in insulin action; type a insulin resistance; leprechaunism; Rabson-Mendenhall syndrome; lipoatrophic diabetes; pancreatectomy; cystic fibrosis; hemochromatosis; fibrocalculous pancreatopathy; endocrinopathies; acromegaly; Cushing's syndrome; glucagonoma; pheochromocytoma; hyperthyroidism; somatostatinoma; aldosteronoma; drug- or chemical-induced diabetes such as from the following drugs: Vacor; Pentamdine; Nicotinic acid; Glucocorticoids; Thyroid hormone; Diazoxide; Adrenergic agonists; Thiazides; Dilantin; and Interferon; pancreatic infections; congential rubella; cytomegalovirus; uncommon forms of immune-mediated diabetes; “stiff-man” syndrome; anti-insulin receptor antibodies; in addition to other genetic syndromes sometimes associated with diabetes which include; for example; Down's syndrome; Klinefelter's syndrome; Turner's syndrome; Wolfram's syndrome; Friedrich's ataxia; Huntington's chorea; Lawrence Moon Beidel syndrome; Myotonic dystrophy; Porphyria; and Prader Willi syndrome; and/or Gestational diabetes mellitus (GDM).

20. The method for preventing, treating, or ameliorating a medical condition of claim 11, wherein the medical condition is selected from the group consisting of: a disorder related to aberrant G-protein coupled signaling; oxytocin-related disorders; neurological disorders; a disorder related to aberrant cell cycle regulation; neurological disorders; anxiety; headache; migraine; manic depression; delirium; severe mental retardation; dyskinesias; neuropathic pain; altered behavior disorders; altered sexual behavior disorders; altered maternal behavior disorders; social behavior disorders; stress-related behavior disorders; feeding and grooming disorders; memory disorders; learning disorders; disorders involving altered ability to establish long term potentiation; sleep disorders; disorders associated with inability to establish or maintain circadian rhythms; balance disorders; perceptive disorders; anorexia; Prader-Willi syndrome; Alzheimer's disease; Parkinson's disease; Huntington's Disease; Tourette Syndrome; psychotic disorders; schizophrenia; mania; dementia; paranoia; depression; obsessive-compulsive disorders; panic disorder disorders involving altered synapse formation; disorders involving altered neurotransmission; demyelinating diseases; ALS; disorders involving altered cognition; disorders involving altered brain homeostasis; disorders involving altered neuronal differentiation or survival; disorders involving the maintenance of an attentive or alert state; disorders involving altered release or synthesis of neurotransmitters such as dopamine, opioid peptides, serotonin, GABA, and glutamate; addictive disorders; disorders involving altered tolerance to opioids; opioid addictions; meningitis; encephalitis; peripheral neuropathies; neoplasia; trauma; congenital malformations; spinal cord injuries; ischemia and infarction; aneurysms; hemorrhages; autism; heart and cardiovascular system disorders; acute heart failure; hypotension; hypertension; endocrinal diseases; growth disorders; obesity; anorexia; HIV infections; cancers; bulimia; asthma; osteoporosis; angina pectoris; myocardial infarction; reproductive disorders; female reproductive system disorders; male reproductive system disorders; mammary tissues disorders; immune-related disorders; kidney function; thymic involution; metabolic disorders; disorders of the pancreas; diabetes mellitus; diabetes; type 1 diabetes; type 2 diabetes; adult onset diabetes; indications related to islet cell transplantation; indications related to pancreatic transplantation; pancreatitis; pancreatic cancer; pancreatic exocrine insufficiency; alcohol induced pancreatitis; maldigestion of fat; maldigestion of protein; hypertriglyceridemia; vitamin b12 malabsorption; hypercalcemia; hypocalcemia; hyperglycemia; ascites; pleural effusions; abdominal pain; pancreatic necrosis; pancreatic abscess; pancreatic pseudocyst; gastrinomas; pancreatic islet cell hyperplasia; multiple endocrine neoplasia type 1 (men 1) syndrome; insulitis; amputations; diabetic neuropathy; pancreatic auto-immune disease; genetic defects of cell function; HNF-1 aberrations (formerly MODY3); glucokinase aberrations (formerly MODY2); HNF-4 aberrations (formerly MODY1); mitochondrial DNA aberrations; genetic defects in insulin action; type a insulin resistance; leprechaunism; Rabson-Mendenhall syndrome; lipoatrophic diabetes; pancreatectomy; cystic fibrosis; hemochromatosis; fibrocalculous pancreatopathy; endocrinopathies; acromegaly; Cushing's syndrome; glucagonoma; pheochromocytoma; hyperthyroidism; somatostatinoma; aldosteronoma; drug- or chemical-induced diabetes such as from the following drugs: Vacor; Pentamdine; Nicotinic acid; Glucocorticoids; Thyroid hormone; Diazoxide; Adrenergic agonists; Thiazides; Dilantin; and Interferon; pancreatic infections; congential rubella; cytomegalovirus; uncommon forms of immune-mediated diabetes; “stiff-man” syndrome; anti-insulin receptor antibodies; in addition to other genetic syndromes sometimes associated with diabetes which include; for example; Down's syndrome; Klinefelter's syndrome; Turner's syndrome; Wolfram's syndrome; Friedrich's ataxia; Huntington's chorea; Lawrence Moon Beidel syndrome; Myotonic dystrophy; Porphyria; and Prader Willi syndrome; and/or Gestational diabetes mellitus (GDM).
